(19) United States
(12) Patent Application Publication
(10) Pub. No.: US 2012/0000300 A1
(43) Pub. Date: Jan. 5, 2012

(54) BODY CONDITION EVALUATION APPARATUS, CONDITION ESTIMATION APPARATUS, STRIDE ESTIMATION APPARATUS, AND HEALTH MANAGEMENT SYSTEM

(76) Inventors: Yoshikazu Sunagawa, Osaka (JP); Hiromu Ueshima, Shiga (JP); Yoshihiro Iijima, Shiga (JP); Yuuki Konno, Shiga (JP)

(21) Appl. No.: 12/720,435

(22) Filed: Mar. 9, 2010

(30) Foreign Application Priority Data
Mar. 10, 2009 (JP) 2009-056977
Sep. 9, 2009 (JP) 2009-207955
Nov. 12, 2009 (JP) 2009-259338

(51) Int. Cl. A61B 5/11

(52) U.S. Cl. 73/865.4

(57) ABSTRACT

A body condition evaluation apparatus measures vicarious movement accompanying predetermined motion, and evaluates a physical condition of a body of a subject by using it as an index. A sensor unit 3L is mounted between a shoulder joint and a cubital joint of a left arm, a sensor unit 3R is mounted between a shoulder joint and a cubital joint of a right arm, a sensor unit 3C is mounted on a breast, and a sensor unit 3W is mounted on a navel. The respective sensors have a triaxial acceleration sensor 13. A movable range of the left arm is obtained on the basis of the sensor 3L, a movable range of the right arm is obtained on the basis of the sensor 3R, displacement widths of an upper body in a front-back direction and a right-left direction are obtained on the basis of the sensor 3C, and displacement widths of a lower back in a front-back direction and a right-left direction are obtained on the basis of the sensor 3W. For example, a difference between the right displacement width and the left displacement width of the upper body when raising the arms indicates a difference between the right vicarious movement and the left vicarious movement, and represents an extent of a defect of the shoulders.

### BACKGROUND OF THE INVENTION

1. Field of the Invention

The present invention relates to a body condition evaluation apparatus and the related arts for evaluating a physical condition of a subject by mounting a sensor on the subject and detecting motion thereof.

2. Description of the Related Art

Patent Document 1 (Japanese Unexamined Patent Application Publication No. 2004-264060) discloses a device which detects posture of a human body by mounting a plurality of sensor boxes (including an acceleration sensor and an angular velocity sensor) on a plurality of parts of the human body.

Patent Document 2 (Japanese Unexamined Patent Application Publication No. 2006-149792) discloses a muscular strength measuring device. This muscular strength measuring device measures muscular strength in the time when predetermined motion is performed. In addition, this muscular strength measuring device detects also vicarious movement accompanying the predetermined motion. And, this muscular strength measuring device processes a measured value of the muscular strength in the time when the predetermined motion is performed as a valid value if the vicarious movement is not detected. In this way, only the muscular strength of a principal muscle performing the predetermined motion is measured and is evaluated. Also, the measurement is carried out under a state in which predetermined parts of the body are restrained. Further, the restraint is also for restricting the vicarious movement.

Patent Document 1 does not disclose the detection of the vicarious movement. Also, in Patent Document 2, the vicarious movement is processed as noise. Further, in Patent Document 2, the cost increases due to restraining the predetermined parts of the body, and it is difficult to measure simply.

### SUMMARY OF THE INVENTION

It is an object of the present invention to provide a body condition evaluation apparatus and the related techniques thereof capable of measuring vicarious movement accompanying predetermined motion, and evaluating physical condition of a body of a subject by using it as an index.

It is an another object of the present invention to provide a body condition evaluation apparatus and the related techniques thereof capable of measuring predetermined motion including vicarious movement and just the vicarious movement itself, subsequently, calculating movement of a principal part by subtracting the vicarious movement by calculation, and evaluating physical condition of a body of a subject on the basis thereof, and further reducing a cost and simply measuring by dispensing with equipment for restraining parts of the body.

It is a further object of the present invention to provide a body condition evaluation apparatus and the related techniques thereof capable of evaluating physical condition of a body of a subject on the basis of a movable range, and a displacement width and/or a trajectory of parts of the body of the subject.

In accordance with a first aspect of the present invention, a body condition evaluation apparatus comprising: a vicarious movement measuring unit operable to measure vicarious movement accompanying predetermined motion of a subject; and an evaluating unit operable to evaluate physical condition of a body of the subject on the basis of information of the vicarious movement measured by said vicarious movement measuring unit.

In accordance with this configuration, the vicarious movement accompanying predetermined motion is measured, and therefore it is possible to evaluate the physical condition (the posture and so on) of the body of the subject by using it as an index.

Even if the physical conditions of the parts of the body such as the muscles and joints are normal, the vicarious movement usually accompanies motion. However, if the vicarious movement is too great, it means that a part (hereinafter referred to as a “principal part”) which originally has to perform motion does not function normally, or there is a part (hereinafter referred to as an “encumbering part”) which encumbers the function of the principal part.

Accordingly, it is possible to determine whether or not the principal part normally functions, or grasp and evaluate the extent of the normal or abnormal on the basis of the extent of the vicarious movement accompanying the predetermined motion. Also, it is possible to determine whether or not the encumbrance by the encumbering part occurs (is within the normal range), or evaluate the extent of the encumbrance, i.e., the encumbering part on the basis of the extent of the vicarious movement. As the encumbrance by the encumbering part becomes greater, the vicarious movement becomes greater so as to absorb and eliminate the encumbrance. On the other hand, as the encumbrance by the encumbering part becomes smaller, the vicarious movement becomes smaller.

Incidentally, in the claims and the specification, the physical condition of the body includes condition of a body, a joint, and bone, and posture, and does not include mental condition.

In this body condition evaluation apparatus, wherein said vicarious movement measuring unit is mounted on the subject so as not to positively restrict the predetermined motion and the vicarious movement.

In accordance with this configuration, since the predetermined motion and the vicarious movement are not restricted, it is possible to measure the vicarious movement under a natural situation, i.e., a situation where the subject is usually put in a regular life. As the result, it is possible to evaluate the condition of the posture of the subject under the natural situation. In contrast, in Patent Document 2, the predetermined parts of the subject are restrained (positively restricted), and therefore this situation is a special situation for measuring.

Incidentally, in the claims and the specification, the terms “not to positively restrict” means that restricting the move of the subject is not the original purpose, such as the constriction by the belt and so on for mounting the sensor such as the vicarious movement measuring unit on the subject.

In this body condition evaluation apparatus, wherein said vicarious movement measuring unit is arranged on a central line which divides the subject into right and left.

In accordance with this configuration, it is possible to effectively measure the vicarious movement. Because, it is possible to measure not only the vicarious movement of the part on the central line but also the bilaterally symmetric vicarious movement with regard to the central line.

For example, said vicarious movement measuring unit is arranged on any one of a breast, a roughly position of a navel, a position between right and left blade bones, and a lower back of the subject.

In the above body condition evaluation apparatus, wherein the predetermined motion includes a first motion and a second motion which is symmetrical to the first motion with regard to a central line which divides the subject into right and left, wherein the vicarious movement includes a first vicarious movement accompanying the first motion and a second vicarious movement accompanying the second motion, and wherein said vicarious movement measuring unit measures the first vicarious movement and the second vicarious movement, said body condition evaluation apparatus further comprising: a difference calculating unit operable to calculate a difference between information of the first vicarious movement and information of the second vicarious movement, wherein the evaluating unit evaluates the physical condition of the body of the subject on the basis of the difference.

In accordance with this configuration, since the difference between the vicarious movements accompanying the bilaterally symmetric motion is obtained, it is possible to determine the defect of any one of the right and left or evaluate the balance of the right and left with regard to the part performing the vicarious movement.

In accordance with a second aspect of the present invention, a body condition evaluation apparatus comprising: a vicarious movement measuring unit operable to measure vicarious movement accompanying predetermined motion of a subject; a motion measuring unit operable to measure the predetermined motion including the vicarious movement; a motion calculating unit operable to calculate motion performed by a part which should originally perform the predetermined motion by subtracting information of the vicarious movement as measured by said vicarious movement measuring unit from information of the predetermined motion as measured by said motion measuring unit; and an evaluating unit operable to evaluate physical condition of a body of the subject on the basis of information of the motion as calculated by said motion calculating unit.

In accordance with this configuration, since the vicarious movement (so to speak, the noise) is subtracted, only the motion by a part (hereinafter referred to as a “principal part”) which originally has to perform the predetermined motion can be extracted, and therefore it is possible to accurately evaluate the principal part.

Also, since the vicarious movement is subtracted by the calculation after performing the predetermined motion including the vicarious movement, it is possible to reduce a cost and simply measure by dispensing with equipment for restraining parts of the body as the prior art.

In this body condition evaluation apparatus, wherein said vicarious movement measuring unit and said motion measuring unit are mounted on the subject so as not to positively restrict the predetermined motion including the vicarious movement.

In accordance with this configuration, since the predetermined motion and the vicarious movement are not restricted, it is possible to measure the predetermined motion and the vicarious movement under a natural situation, i.e., a situation where the subject is usually put in a regular life. As the result, it is possible to evaluate the condition of the posture of the subject under the natural situation. In contrast, in Patent Document 2, the predetermined parts of the subject are restrained, and therefore this situation is a special situation for measuring.

In this body condition evaluation apparatus, wherein said vicarious movement measuring unit and said motion measuring unit are arranged at positions different from each other on a central line which divides the subject into right and left.

In accordance with this configuration, it is possible to effectively measure the vicarious movement. Because, it is possible to measure not only the motion of the part on the central line (the predetermined motion and the vicarious movement) but also the bilaterally symmetric motion with regard to the central line (the predetermined motion and the vicarious movement).

For example, said vicarious movement measuring unit is arranged on any one of a breast, a roughly position of a navel, a position between right and left blade bones, and a lower back of the subject, and wherein said motion measuring unit is arranged at any one of a roughly position of the navel and the lower back of the subject when said vicarious movement measuring unit is arranged on either the breast or the position between the right and left blade bones, and is arranged on any one of the breast and a position between the right and left blade bones of the subject when said vicarious movement measuring unit is arranged at either the roughly position of the navel or the lower back.

In the above body condition evaluation apparatus, wherein the predetermined motion includes a first motion and a second motion which is symmetrical to the first motion with regard to a central line which divides the subject into right and left, wherein the vicarious movement includes a first vicarious movement accompanying the first motion and a second vicarious movement accompanying the second motion, and wherein said vicarious movement measuring unit measures the first vicarious movement and the second vicarious movement, wherein said motion measuring unit measures the first motion including the first vicarious movement and the second motion including the second vicarious movement, and wherein said motion calculating unit calculate motion performed by a part which should originally perform the first motion by subtracting information of the first vicarious movement from information of the first motion, and motion performed by a part which should originally perform the second motion by subtracting information of the second vicarious movement from information of the second motion, said body condition evaluation apparatus further comprising: a difference calculating unit operable to calculate a difference between the motion performed by the part which should originally perform the first motion and the motion performed by the part which should originally perform the second motion, wherein the evaluating unit evaluates the physical condition of the body of the subject on the basis of the difference as calculated by said difference calculating unit.

In accordance with this configuration, since the difference between the bilaterally symmetric movements which are performed by the principal parts is obtained, it is possible to determine the defect of any one of the right and left, or evaluate the balance of the right and left with regard to the principal parts.

In accordance with a third aspect of the present invention, a body condition evaluation apparatus capable of evaluating physical condition of a body of a subject which has symmetrical structure with regard to a central line, comprising: a first detecting unit and a second detecting unit configured to be mounted on two parts which are symmetrical to each other with regard to the central line, and detect motions of the parts; a third detecting unit configured to be mounted on a part on the central line, and detect motion of the part; a first calculating unit operable to calculate an amount of change of the motion of the corresponding part which is detected by said first detecting unit when the two parts change from a first state to a second state respectively; a second calculating unit operable to calculate an amount of change of the motion of the corresponding part which is detected by said second detecting unit when the two parts change from the first state to the second state respectively; a third calculating unit operable to calculate a maximum value and/or a trajectory of the motion of the part which is detected by said third detecting unit from a start to a finish of a predetermined motion which is performed by the subject; and an evaluating unit operable to evaluate the physical condition of the body of the subject on the basis of the amount of the change calculated by said first calculating unit, the amount of the change calculated by said second calculating unit, and the maximum value and/or the trajectory calculated by said third calculating unit.

In accordance with this configuration, the movable range of the part of the subject (the amount of the change when the part changes from the first state to the second state), and the displacement width of the part (the maximum value of the motion of the part from the start to the finish of the predetermined motion) and/or the trajectory of the motion of the part are detected, and therefore it is possible to analyze and evaluate the physical condition (the posture and so on) of the body of the subject on the basis of them.

In this body condition evaluation apparatus, wherein the subject is a human, wherein said first detecting unit is mounted between a shoulder joint and a cubital joint on a left arm of the human, wherein said second detecting unit is mounted between a shoulder joint and a cubital joint on a right arm of the human, and wherein the third detecting unit is mounted near a pelvis on a side of a belly or a side of a back of the human.

In accordance with this configuration, the movable range of the left arm is measured on the basis of the output of the first detecting unit, the movable range of the right arm is measured on the basis of the output of the second detecting unit, and therefore it is possible to evaluate the inclination of the shoulders on the basis of them. Also, it is possible to deduce the position of the load and the tilt of the pelvis by measuring the displacement widths and/or the trajectory of the pelvis in the front, back, right, and left directions on the basis of the third detecting unit.

In this body condition evaluation apparatus, wherein said evaluating unit comprising: a first comparing unit operable to compare the amount of the change calculated by said first calculating unit and the amount of the change calculated by said second calculating unit to determine which of a right shoulder and a left shoulder of the subject is higher than the other; a second comparing unit operable to compare the maximum value in a right direction and the maximum value in a left direction of the subject as calculated by said third calculating unit to determine to which of a right side and a left side a load of the subject is applied; a third comparing unit operable to compare the maximum value in a forward-tilt direction of the subject as calculated by said third calculating unit and a predetermined value to determine to which of a condition of a forward tilt and a condition of a backward tilt the pelvis belongs; and a unit operable to evaluate the physical condition of the body of the subject on the basis of the comparison result by said first comparing unit, the comparison result by said second comparing unit, and the comparison result by said third comparing unit.

In accordance with this configuration, it is possible to evaluate the physical condition of the body of the subject on the basis of the inclination of the shoulders, the position of the load, and the tilt of the pelvis.

The above body condition evaluation apparatus further comprising: a change displaying unit operable to display processes of the motions of the two parts which are symmetrical to each other with regard to the central line, the maximum value which changes from moment to moment with regarding to the part on the central line, and/or the trajectory of the motion of the part on the central line with an image on a display device.

In accordance with this configuration, the subject such as the human subject can monitor the movement of the his/her own measured parts on a real-time basis, and the measurer such as a doctor can monitor the movement of the measured parts of the subject on a real-time basis.

The above body condition evaluation apparatus further comprising: a first guiding unit operable to guide a motion from the first state to the second state with an image; and a second guiding unit operable to guide the predetermined motion with an image.

In accordance with this configuration, since the motion which is required to measure is shown with the image, it is possible to instruct all the subjects the uniform motion. Also, the subject can easily recognize the motion which he/she has to perform.

The above body condition evaluation apparatus further comprising: a correction exercise displaying unit operable to display guidance of exercise for correcting the physical condition of the body indicated by said evaluating unit with an image on a display device.

In accordance with this configuration, in addition to evaluating the physical condition of the body, the exercise for correcting it is shown, whereby it is possible to seamlessly link the measurement and evaluation of the physical condition of the body, and the correction exercise, and therefore it is convenient for the subject.

In the above body condition evaluation apparatus, wherein arbitrary one of an acceleration sensor, an angular velocity sensor, a direction sensor, and an inclination sensor can optionally be employed as said first detecting unit, said second detecting unit, or said third detecting unit.

In accordance with this configuration, it is possible to use the suitable detecting unit depending on the specifications of the body condition evaluation apparatus.

In accordance with a fourth aspect of the present invention, a body condition evaluation apparatus capable of evaluating physical condition of a body of a subject which has symmetrical structure with regard to a central line, comprising: a first detecting unit and a second detecting unit configured to be mounted on two parts which are symmetrical to each other with regard to the central line, and detect motions of the parts; a first change amount calculating unit operable to calculate an amount of change of the motion of the corresponding part which is detected by said first detecting unit when the two parts change from a first state to a second state respectively; a second change amount calculating unit operable to calculate an amount of change of the motion of the corresponding part which is detected by said second detecting unit when the two parts change from the first state to the second state respectively; and an evaluating unit operable to evaluate the physical condition of the body of the subject on the basis of the amount of the change calculated by said first change amount calculating unit, and the amount of the change calculated by said second change amount calculating unit.

In accordance with this configuration, the movable range of the part of the subject (the amount of the change when the part changes from the first state to the second state) is detected, and therefore it is possible to evaluate the physical condition on the basis thereof.

In accordance with a fifth aspect of the present invention, a body condition evaluation apparatus capable of evaluating physical condition of a body of a subject which has symmetrical structure with regard to a central line, comprising: a detecting unit configured to be mounted on a part on the central line, and detect motion of the part; a maximum value calculating unit operable to calculate a maximum value of the motion of the part which is detected by said detecting unit from a start to a finish of a predetermined motion which is performed by the subject; and an evaluating unit operable to evaluate the physical condition of the body of the subject on the basis of the maximum value calculated by said maximum value calculating unit.

In accordance with this configuration, the displacement width of the part (the maximum value of the motion of the part from the start to the finish of the predetermined motion) is detected, and therefore it is possible to analyze and evaluate the physical condition of the body of the subject on the basis thereof.

In accordance with a sixth aspect of the present invention, a body condition evaluation apparatus capable of evaluating physical condition of a body of a subject which has symmetrical structure with regard to a central line, comprising: a detecting unit configured to be mounted on a part on the central line, and detect motion of the part; a trajectory calculating unit operable to calculate a trajectory of the motion of the part which is detected by said detecting unit from a start to a finish of a predetermined motion which is performed by the subject; and an evaluating unit operable to evaluate the physical condition of the body of the subject on the basis of the trajectory calculated by said trajectory calculating unit.

In accordance with this configuration, the trajectory of the motion of the part (the trajectory of the motion of the part from the start to the finish of the predetermined motion) is detected, and therefore it is possible to analyze and evaluate the physical condition of the body of the subject on the basis thereof.

In accordance with a seventh aspect of the present invention, a condition deducing apparatus capable of deducing a part whose muscle is apt to go tight and a part on which fat is apt to be put, comprising: a measuring unit operable to measure condition of shoulders of a subject; a determining unit operable to determine the condition of the shoulders on the basis of the measurement result; and a deducing unit operable to deduce that muscles of the left shoulder and a right flank are apt to go tight and fat is apt to be put on the right shoulder and a left flank when said determining unit determines that the shoulders incline upward to the left, and deduce that muscles of the right shoulder and the left flank are apt to go tight and fat is apt to be put on the left shoulder and the right flank when said determining unit determines that the shoulders incline upward to the right.

In accordance with this configuration, it is possible to simply deduce the parts on which fat is apt to be put and the parts whose muscles are apt to go tight in the shoulder and the flank only by measuring the condition of the shoulders.

In accordance with a eighth aspect of the present invention, a condition deducing apparatus capable of deducing a part of a body whose muscle is apt to go tight and a part of the body on which fat is apt to be put, comprising: a measuring unit operable to measure condition of a pelvis of a subject; a determining unit operable to determine the condition of the pelvis on the basis of the measurement result; and a deducing unit operable to deduce that muscles of a left waist portion and a right flank are apt to go tight and fat is apt to be put on a right waist portion and a left flank when said determining unit determines that the pelvis inclines upward to the right, and deduce that muscles of the right waist portion and the left flank are apt to go tight and fat is apt to be put on the left waist portion and the right flank when said determining unit determines that the pelvis inclines upward to the left.

In accordance with this configuration, it is possible to simply deduce the parts on which fat is apt to be put and the parts whose muscles are apt to go tight in the waist and the flank only by measuring the condition of the pelvis.

In accordance with a ninth aspect of the present invention, a condition deducing apparatus capable of deducing a part of a body whose muscle is apt to go tight and a part of the body on which fat is apt to be put, comprising: a measuring unit operable to measure condition of a pelvis of a subject; a determining unit operable to determine the condition of the pelvis on the basis of the measurement result; and a deducing unit operable to deduce that muscles of a back and a front side of a thigh are apt to go tight and fat is apt to be put on a belly, a buttock, and a back side of the thigh when said determining unit determines that the pelvis tilts forward, and deduce that muscles of a breast, the buttock, and the back side of the thigh are apt to go tight and fat is apt to be put on the back and the front side of the thigh when said determining unit determines that the pelvis tilts backward.

In accordance with this configuration, it is possible to simply deduce the parts on which fat is apt to be put and the parts whose muscles are apt to go tight in the breast, the back, the belly, the buttocks, and the front sides and the back sides of the thighs only by measuring the condition of the pelvis.

In accordance with a tenth aspect of the present invention, a condition deducing apparatus capable of deducing a part of a body whose muscle is apt to go tight and a part of the body on which fat is apt to be put, comprising: a measuring unit operable to measure condition of great trochanters of a subject; a determining unit operable to determine the condition of the great trochanters on the basis of the measurement result; and a deducing unit operable to deduce that muscles of a left waist portion and an inner side of a right thigh are apt to go tight and fat is apt to be put on a right waist portion and an inner side of a left thigh when said determining unit determines that the great trochanters incline upward to the left, and deduce that muscles of the right waist portion and the inner side of the left thigh are apt to go tight and fat is apt to be put on the left waist portion and the inner side of the right thigh when said determining unit determines that the great trochanters incline upward to the right.

In accordance with this configuration, it is possible to simply deduce the parts on which fat is apt to be put and the parts whose muscles are apt to go tight in the waist and the inner side of the thigh only by measuring the condition of the great trochanters.

In accordance with an eleventh aspect of the present invention, a stride deducing apparatus capable of deducing a stride of a subject, comprising: a first measuring unit operable to measure condition of a pelvis of the subject; a determining unit operable to determine the condition of the pelvis on the basis of the measurement result; and a deducing unit operable to deduce that the right stride is large when said determining unit determines that the pelvis inclines upward to the right, and deduce that the left stride is large when said determining unit determines that the pelvis inclines upward to the left.

In accordance with this configuration, it is possible to simply deduce the stride only by measuring the condition of the pelvis.

This stride deducing apparatus further comprising: a second measuring unit operable to measure condition of great trochanters of the subject, wherein said determining unit determines the condition of the great trochanters on the basis of the measurement result, and wherein even if said determining unit determines that the condition of the pelvis is normal, said deducing unit deduces that the right stride is large when the determining unit determines that the great trochanters incline upward to the left, and deduces that the left stride is large when the determining unit determines that the great trochanters incline upward to the right.

In accordance with this configuration, since the condition of the great trochanters is reflected, it is possible to deduce the stride more accurately.

In accordance with a twelfth aspect of the present invention, a health management system comprising: a posture measuring apparatus operable to measure posture of a subject; a predetermined first terminal operable to be accessed by the subject; and a server configured to connect with said posture measuring apparatus and said predetermined first terminal through a network, wherein said predetermined first terminal comprising: a measuring unit operable to measure body information and/or behavior information of the subject, wherein said posture measuring apparatus transmits data with regard to the posture of the subject as measured to said server through the network, wherein said predetermined first terminal transmits the body information and/or the behavior information of the subject as measured to said server through the network, and wherein said server transmits information created on the basis of the data with regard to the posture of the subject and the body information and/or the behavior information of the subject as received to said predetermined first terminal through the network.

In accordance with this configuration, the information of the posture of the subject as well as the body information such as the weight and the behavior information such as the number of steps are transmitted to the server. Thus, the specialists of medical care, health, and so on of the operating entity of the server can more finely analyze and evaluate in comparison with the analysis and the evaluation based only on the body information and behavior information, and analyze and evaluate on the basis of the physical condition (posture) of the body. And, since the results of these analysis and evaluation are supplied the predetermined terminal, the subject can more finely carry out the health management based on the posture (the physical condition of the body).

This health management system further comprising: a predetermined second terminal configured to be set up in a medical institution, and connect with said server through the network, wherein said predetermined second terminal accesses said server to display or acquire the data with regard to the posture of the subject and the body information and/or the behavior information of the subject which are received by said server, and transmits information created on the basis of the data with regard to the posture of the subject and the body information and/or the behavior information of the subject as displayed or acquired to said predetermined first terminal.

In accordance with this configuration, since the doctor of the medical institution can refer the daily body information, the daily behavior information, and the posture information of the subject as well as the condition of the subject (patient) at the hospital visiting, the doctor can more finely exactly diagnose and examine. It is generally believed that it is difficult for a doctor other than an orthopedic surgeon and so on which treat a disorder of a backbone, bones of extremities, a joint, and a muscle system to measure and acquire information of posture of a patient. In accordance with the present invention, even such doctor can easily acquire the information of the posture to utilize for the diagnosis and the creation of the exercise prescription. As a result, the subject can receive the daily life guidance and the exercise prescription based on more precise diagnosis from the doctor and so on via the second terminal and the first terminal.

In accordance with a thirteenth aspect of the present invention, a body condition evaluation method comprising: a vicarious movement measuring step operable to measure vicarious movement accompanying predetermined motion of a subject; and an evaluating step operable to evaluate physical condition of a body of the subject on the basis of information of the vicarious movement measured by said vicarious movement measuring step.

In accordance with a fourteenth aspect of the present invention, a computer-readable medium stores a computer program for making a computer execute the body condition evaluation method according to the above thirteenth aspect.

In accordance with the thirteenth and fourteenth aspects, the same advantage as the body condition evaluation apparatus according to the above first aspect can be gotten.

In accordance with a fifteenth aspect of the present invention, a body condition evaluation method comprising: a vicarious movement measuring step operable to measure vicarious movement accompanying predetermined motion of a subject; a motion measuring step operable to measure the predetermined motion including the vicarious movement; a motion calculating step operable to calculate motion performed by a part which should originally perform the predetermined motion by subtracting information of the vicarious movement as measured by said vicarious movement measuring step from information of the predetermined motion as measured by said motion measuring step; and an evaluating step operable to evaluate physical condition of a body of the subject on the basis of information of the motion as calculated by said motion calculating step.

In accordance with a sixteenth aspect of the present invention, a computer-readable medium stores a computer program for making a computer execute the body condition evaluation method according to the above fifteenth aspect.

In accordance with the fifteenth and sixteenth aspects, the same advantage as the body condition evaluation apparatus according to the above second aspect can be gotten.

In accordance with a seventeenth aspect of the present invention, a body condition evaluation method for evaluating physical condition of a body of a subject using information from a first detecting unit, a second detecting unit, and a third detecting unit mounted on the subject which has symmetrical structure with regard to a central line, the first and second detecting units being mounted on two parts which are symmetrical to each other with regard to the central line, and detecting motions of the parts, the third detecting unit being mounted on a part on the central line, and detecting motion of the part, comprising: a first calculating step operable to calculate an amount of change of the motion of the corresponding part which is detected by said first detecting unit when the two parts change from a first state to a second state respectively; a second calculating step operable to calculate an amount of change of the motion of the corresponding part which is detected by said second detecting unit when the two parts change from the first state to the second state respectively; a third calculating step operable to calculate a maximum value and/or a trajectory of the motion of the part which is detected by said third detecting unit from a start to a finish of a predetermined motion which is performed by the subject; and an evaluating step operable to evaluate the physical condition of the body of the subject on the basis of the amount of the change calculated by said first calculating step, the amount of the change calculated by said second calculating step, and the maximum value and/or the trajectory calculated by said third calculating step.

In accordance with a eighteenth aspect of the present invention, a computer-readable medium stores a computer program for making a computer execute the body condition evaluation method according to the above seventeenth aspect.

In accordance with the seventeenth and eighteenth aspects, the same advantage as the body condition evaluation apparatus according to the above third aspect can be gotten.

In accordance with a nineteenth aspect of the present invention, a body condition evaluation method for evaluating physical condition of a body of a subject using information from a first detecting unit and a second detecting unit mounted on the subject which has symmetrical structure with regard to a central line, the first and second detecting units being mounted on two parts which are symmetrical to each other with regard to the central line, and detecting motions of the parts, and detecting motion of the part, comprising: a first change amount calculating step operable to calculate an amount of change of the motion of the corresponding part which is detected by said first detecting unit when the two parts change from a first state to a second state respectively; a second change amount calculating step operable to calculate an amount of change of the motion of the corresponding part which is detected by said second detecting unit when the two parts change from the first state to the second state respectively; and an evaluating step operable to evaluate the physical condition of the body of the subject on the basis of the amount of the change calculated by said first change amount calculating step, and the amount of the change calculated by said second change amount calculating step.

In accordance with a twentieth aspect of the present invention, a computer-readable medium stores a computer program for making a computer execute the body condition evaluation method according to the above nineteenth aspect.

In accordance with the nineteenth and twentieth aspects, the same advantage as the body condition evaluation apparatus according to the above fourth aspect can be gotten.

In accordance with a twenty-first aspect of the present invention, a body condition evaluation method for evaluating physical condition of a body of a subject using information from a detecting unit mounted on the subject which has symmetrical structure with regard to a central line, the detecting unit being mounted on a part on the central line, and detecting motion of the part, comprising: a maximum value calculating step operable to calculate a maximum value of the motion of the part which is detected by said detecting unit from a start to a finish of a predetermined motion which is performed by the subject; and an evaluating step operable to evaluate the physical condition of the body of the subject on the basis of the maximum value calculated by said maximum value calculating step.

In accordance with a twenty-second aspect of the present invention, a computer-readable medium stores a computer program for making a computer execute the body condition evaluation method according to the above twenty-first aspect.

In accordance with the twenty-first and twenty-second aspects, the same advantage as the body condition evaluation apparatus according to the above fifth aspect can be gotten.

In accordance with a twenty-third aspect of the present invention, a body condition evaluation method for evaluating physical condition of a body of a subject using information from a detecting unit mounted on the subject which has symmetrical structure with regard to a central line, the detecting unit being mounted on a part on the central line, and detecting motion of the part, comprising: a trajectory calculating step operable to calculate a trajectory of the motion of the part which is detected by said detecting unit from a start to a finish of a predetermined motion which is performed by the subject; and an evaluating step operable to evaluate the physical condition of the body of the subject on the basis of the trajectory calculated by said trajectory calculating step.

In accordance with a twenty-fourth aspect of the present invention, a computer-readable medium stores a computer program for making a computer execute the body condition evaluation method according to the above twenty-third aspect.

In accordance with the twenty-third and twenty-fourth aspects, the same advantage as the body condition evaluation apparatus according to the above sixth aspect can be gotten.

In accordance with a twenty-fifth aspect of the present invention, a condition deducing method capable of deducing a part whose muscle is apt to go tight and a part on which fat is apt to be put, comprising: a measuring step operable to measure condition of shoulders of a subject; a determining step operable to determine the condition of the shoulders on the basis of the measurement result; and a deducing step operable to deduce that muscles of the left shoulder and a right flank are apt to go tight and fat is apt to be put on the right shoulder and a left flank when said determining step determines that the shoulders incline upward to the left, and deduce that muscles of the right shoulder and the left flank are apt to go tight and fat is apt to be put on the left shoulder and the right flank when said determining step determines that the shoulders incline upward to the right.

In accordance with a twenty-sixth aspect of the present invention, a computer-readable medium stores a computer program for making a computer execute the body condition evaluation method according to the above twenty-fifth aspect.

In accordance with the twenty-fifth and twenty-sixth aspects, the same advantage as the condition deducing apparatus according to the above seventh aspect can be gotten.

In accordance with a twenty-seventh aspect of the present invention, a condition deducing method capable of deducing a part whose muscle is apt to go tight and a part on which fat is apt to be put, comprising: a measuring step operable to measure condition of a pelvis of a subject; a determining step operable to determine the condition of the pelvis on the basis of the measurement result; and a deducing step operable to deduce that muscles of a left waist portion and a right flank are apt to go tight and fat is apt to be put on a right waist portion and a left flank when said determining step determines that the pelvis inclines upward to the right, and deduce that muscles of the right waist portion and the left flank are apt to go tight and fat is apt to be put on the left waist portion and the right flank when said determining step determines that the pelvis inclines upward to the left.

In accordance with a twenty-eighth aspect of the present invention, a computer-readable medium stores a computer program for making a computer execute the body condition evaluation method according to the above twenty-seventh aspect.

In accordance with the twenty-seventh and twenty-eighth aspects, the same advantage as the condition deducing apparatus according to the above eighth aspect can be gotten.

In accordance with a twenty-ninth aspect of the present invention, a condition deducing method capable of deducing a part whose muscle is apt to go tight and a part on which fat is apt to be put, comprising: a measuring step operable to measure condition of a pelvis of a subject; a determining step operable to determine the condition of the pelvis on the basis of the measurement result; and a deducing step operable to deduce that muscles of a back and a front side of a thigh are apt to go tight and fat is apt to be put on a belly, a buttock, and a back side of the thigh when said determining step determines that the pelvis tilts forward, and deduce that muscles of a breast, the buttock, and the back side of the thigh are apt to go tight and fat is apt to be put on the back and the front side of the thigh when said determining step determines that the pelvis tilts backward.

In accordance with a thirtieth aspect of the present invention, a computer-readable medium stores a computer program for making a computer execute the body condition evaluation method according to the above twenty-ninth aspect.

In accordance with the twenty-ninth and thirtieth aspects, the same advantage as the condition deducing apparatus according to the above ninth aspect can be gotten.

In accordance with a thirty-first aspect of the present invention, a condition deducing method capable of deducing a part whose muscle is apt to go tight and a part on which fat is apt to be put, comprising: a measuring step operable to measure condition of great trochanters of a subject; a determining step operable to determine the condition of the great trochanters on the basis of the measurement result; and a deducing step operable to deduce that muscles of a left waist portion and an inner side of a right thigh are apt to go tight and fat is apt to be put on a right waist portion and an inner side of a left thigh when said determining step determines that the great trochanters incline upward to the left, and deduce that muscles of the right waist portion and the inner side of the left thigh are apt to go tight and fat is apt to be put on the left waist portion and the inner side of the right thigh when said determining step determines that the great trochanters incline upward to the right.

In accordance with a thirty-second aspect of the present invention, a computer-readable medium stores a computer program for making a computer execute the body condition evaluation method according to the above thirty-first aspect.

In accordance with the thirty-first and thirty-second aspects, the same advantage as the condition deducing apparatus according to the above tenth aspect can be gotten.

In accordance with a thirty-third aspect of the present invention, a stride deducing method capable of deducing a stride of a subject, comprising: a first measuring step operable to measure condition of a pelvis of the subject; a determining step operable to determine the condition of the pelvis on the basis of the measurement result; and a deducing step operable to deduce that the right stride is large when said determining step determines that the pelvis inclines upward to the right, and deduce that the left stride is large when said determining step determines that the pelvis inclines upward to the left.

In accordance with a thirty-fourth aspect of the present invention, a computer-readable medium stores a computer program for making a computer execute the body condition evaluation method according to the above thirty-third aspect.

In accordance with the thirty-third and thirty-fourth aspects, the same advantage as the stride deducing apparatus according to the above eleventh aspect can be gotten.

In accordance with a thirty-fifth aspect of the present invention, a health management method using a posture measuring apparatus operable to measure posture of a subject; a predetermined first terminal operable to be accessed by the subject; and a server configured to connect with said posture measuring apparatus and said predetermined first terminal through a network, comprising: a step operable to measure body information and/or behavior information of the subject by said predetermined first terminal; a step operable to transmit data with regard to the posture of the subject as measured to said server through the network by said posture measuring apparatus; a step operable to transmit the body information and/or the behavior information of the subject as measured to said server through the network by said predetermined first terminal; and a step operable to transmit information created on the basis of the data with regard to the posture of the subject and the body information and/or the behavior information of the subject as received to said predetermined first terminal through the network by said server.

In accordance with a thirty-sixth aspect of the present invention, a computer-readable medium stores a computer program for making a computer execute the process step which the predetermined first terminal of the health management method according to the above thirty-fifth aspect performs.

In accordance with a thirty-seventh aspect of the present invention, a computer-readable medium stores a computer program for making a computer execute the process step which the posture measuring apparatus of the health management method according to the above thirty-fifth aspect performs.

In accordance with a thirty-eighth aspect of the present invention, a computer-readable medium stores a computer program for making a computer execute the process step which the server of the health management method according to the above thirty-fifth aspect performs.

In accordance with the thirty-fifth, thirty-sixth, thirty-seventh, and thirty-eighth aspects, the same advantage as the health management system according to the above twelfth aspect can be gotten.

The novel features of the invention are set forth in the appended claims. The invention itself, however, as well as other features and advantages thereof, will be best understood by reading the detailed description of specific embodiments in conjunction with the accompanying drawings.

### BRIEF DESCRIPTION OF THE DRAWINGS

FIG. 1 is an explanatory view showing the arrangement of sensor units 3R, 3L, 3C, and 3W of a body condition evaluation system in accordance with an embodiment of the present invention.

FIG. 2 is a view showing the electric configuration of the body condition evaluation system in accordance with the embodiment of the present invention.

FIG. 3 is an explanatory view showing angles which are calculated based on acceleration data from the sensor unit 3.

FIG. 4 is an explanatory view showing posture patterns A-[1] and A-[2].

FIG. 5 is an explanatory view showing posture patterns C-[1] and C-[2].

FIG. 6 is an explanatory view showing a backward warped back.

FIG. 7 is an explanatory view showing a stoop.

FIG. 8 is an explanatory view showing a first motion (arm raising).

FIG. 9 is an explanatory view showing a second motion (forearm rotating).

FIG. 10 is an explanatory view showing a third motion (lateral bending).

FIG. 11 is an explanatory view showing a fourth motion (forward bending).

FIG. 12 is an explanatory view showing a fifth motion (backward bending).

FIG. 13 is an explanatory view showing a sixth motion (thigh raising).

FIG. 14 is a view showing the communication procedure among a PC 7, an MCU 39 of a wireless communication unit 37, and an MCU 11 of the sensor unit 3.

FIG. 15 is a flow chart showing a measuring process by the PC 7.

FIG. 16 is a flow chart showing a classifying process by the PC 7.

FIG. 17 is a view showing an example of a total result screen (the pattern A-[1]).

FIG. 18 is a view showing an example of an exercise prescription screen (stretching).

FIG. 19 is a view showing an example of an exercise prescription screen (conditioning).

FIG. 20 is a view showing an example of a detailed result screen.

FIG. 21 is a view showing an example of a printed result (the pattern A-[1]).

FIG. 22 is a view showing the list of parameters which are used for a detailed evaluation.

FIG. 23 is an explanatory view showing posture barometers for the detailed evaluation.

FIG. 24(a) is an explanatory view showing the detailed evaluation based on the parameters [symbol] , [symbol] , and [symbol] of FIG. 22. FIG. 24(b) is an explanatory view showing conditions 1 to 6 of FIG. 24(a).

FIG. 25 is a flow chart showing an example of a detailed evaluation process which is performed in step S330 of FIG. 16.

FIG. 26 is a view showing a first displayed example of a total result screen in accordance with a first modification example of the embodiment of the present invention.

FIG. 27 is a view showing a second displayed example of the total result screen in accordance with the first modification example.

FIG. 28 is a view showing an example of a printed result in accordance with the first modification example.

FIG. 29 is a flow chart showing an evaluation process by the PC 7 in accordance with the first modification example.

FIG. 30 is a flow chart showing a detailed evaluation process in step S650 of FIG. 29.

FIG. 31(a) is a flow chart showing a four-scale evaluation process in steps S752, S756, S760, S762, S770, S778, and S782 of FIG. 30. FIG. 31(b) is a flow chart showing the four-scale evaluation process in steps S766 and S774 of FIG. 30.

FIG. 32 is a view showing the entire configuration of a health management system in accordance with a second modification example of the embodiment of the present invention.

FIG. 33 is a view showing the entire configuration of a health management terminal 97 of FIG. 32.

FIG. 34(a) is a view showing the electric configurations of a computer 131 and an antenna unit 132 of FIG. 33 FIG. 34(b) is a view showing the electric configuration of a pedometer 135 of FIG. 33. FIG. 34(c) is a view showing the electric configuration of a weight scale 138 of FIG. 33.

FIG. 35(a) is a view showing the electric configuration of a sphygmomanometer 137 of FIG. 33. FIG. 35(b) is a view showing the electric configuration of a mat type controller 140 of FIG. 33.

FIG. 36 is a flowchart showing the overall process flow by a processor 142 of FIG. 34(a).

FIG. 37 is a view showing the communication procedure among the processor 142 of FIG. 34(a), an MCU 146 of FIG. 34(a), and a node (an MCU 152 of the pedometer 135) of FIG. 34(b) (the login procedure).

FIG. 38 is a view showing the communication procedure among the processor 142 of FIG. 34(a), the MCU 146 of FIG. 34(a), and a node (the MCU 152 of the pedometer 135 of FIG. 34(b), an MCU 158 of the weight scale 138 of FIG. 34(c), or an MCU 165 of the sphygmomanometer of FIG. 35(a)) (the data transfer procedure).

FIG. 39 is a flowchart showing a mail process (an exercise menu acquisition process) in step S1017 of FIG. 36.

FIG. 40 is a view showing the communication procedure among the processor 142 of FIG. 34(a), the MCU 146 of FIG. 34(a), and a node (the mat type controller 140 of FIG. 35(b)) (the data transfer procedure).

FIG. 41 is a view showing an example of a map screen 173 displayed on a monitor 134 of FIG. 33.

### DESCRIPTION OF THE PREFERRED EMBODIMENTS

In what follows, an embodiment of the present invention will be explained in conjunction with the accompanying drawings. Meanwhile, like references indicate the same or functionally similar elements throughout the drawings, and therefore redundant explanation is not repeated.

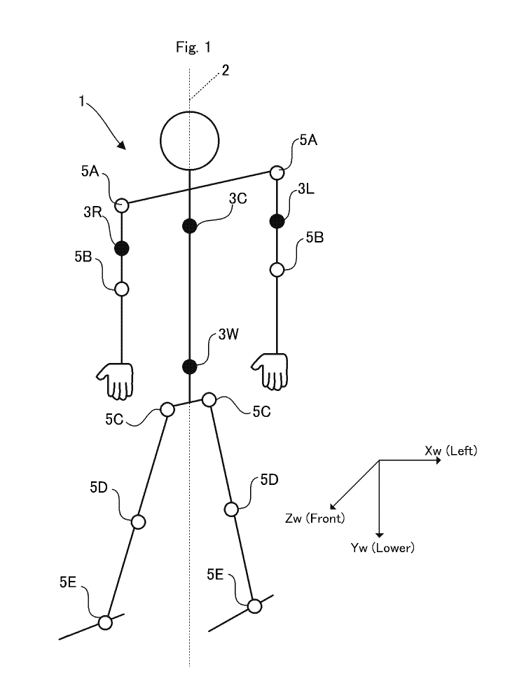

FIG. 1 is an explanatory view showing the arrangement of sensor units 3R, 3L, 3C, and 3W of a body condition evaluation system in accordance with an embodiment of the present invention. Referring to FIG. 1, joints 5A, 5B, 5C, 5D, and 5E of a subject 1 are schematically shown. The sensor unit 3R is arranged between a shoulder joint 5A and a cubital joint 5B of a right arm. The sensor unit 3L is arranged between a shoulder joint 5A and a cubital joint 5B of a left arm. The sensor unit 3C is arranged on a central line 2 between right and left blade bones of a back of the subject 1 (behind a breast). The sensor unit 3W is arranged on the central line 2 of the subject 1 at a roughly position of a dimple of a lower back (a lumbar triangle) Or, the sensor unit 3W is arranged on the central line 2 of the subject 1, and so that it is closely in contact with a pelvis via the lower back.

A local coordinate system (X1, Y1, Z1) is assigned to each of the sensor units 3R, 3L, 3C, and 3W. Also, it is assumed that the subject 1 is located in a reference coordinate system (a world coordinate system) (Xw, Yw, Zw).

Incidentally, the sensor units 3R, 3L, 3C, and 3W are referred to as the “sensor units 3” in the case where they need not be distinguished.

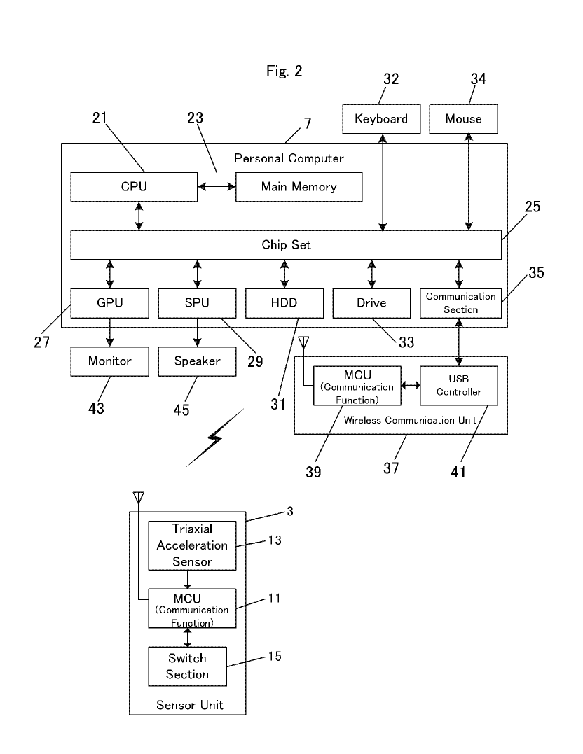

FIG. 2 is a view showing the electric configuration of the body condition evaluation system in accordance with the embodiment of the present invention. Referring to FIG. 2, the body condition evaluation system is provided with a personal computer 7, and the four sensor units 3 (only one is shown in the figure). The personal computer 7 is provided with a CPU (Central Processing Unit) 21, a main memory 23, a chip set 25, a GPU (Graphics Processing Unit) 27, an SPU (Sound Processing Unit) 29, an HDD (Hard Disk Drive) 31, a drive 33, and a communication section 35.

The CPU 21 performs various operations (processes shown by flowcharts to be hereinafter described, and displaying processes of various screen to be hereinafter described) by executing computer programs stored in the HDD 31. The main memory 23 is a high speed memory capable of reading and writing directly by the CPU 21. The GPU 27 performs graphics processing to supply a monitor 43 with a video signal. The SPU 29 performs sound processing to supply a speaker 45 with an audio signal. The HDD 31 is an auxiliary storage device for storing computer programs such as OS and application software, and data to be processed by them. The drive 33 reads data from a removable recording medium and writes data therein. The removable recording medium may store the programs for performing the processes shown by the flowcharts as described below, and the programs for generating the screens, and whereby they can be installed on the PC 7. Needless to say, these programs may be distributed through a network. The communication section 35 includes a LAN card, a USB controller, and so on (not shown in the figure) serving to make a connection with a network, and whereby controls the communication.

Incidentally, the HDD 31, the main memory 23, and the removable recording medium are cited as an example of a recording medium. The removable recording medium includes, for example, a flexible disk, an optical disk such as a CD (including CD-ROM, Video-CD) and a DVD (including DVD-Video, DVD-ROM, DVD-RAM), a magneto-optical disk, and a memory card, a memory cartridge, a USB memory, and so on having a semiconductor memory. Also, as necessary, an SSD (Solid State Drive) may be employed instead of the HDD 31 or together with the HDD 31.

Such functional units as the CPU 21, GPU 27, SPU 29, HDD 31, the drive 33, the communication section 35, the keyboard 32, and the mouse 34 are connected to the chip set 25. The chip set 25 manages data transfer between the functional units connected thereto.

Also, a wireless communication unit 37 is connected with the communication section 35. The wireless communication unit 37 includes an MCU 39 with a communication function and a USB controller 41. The USB controller 41 is connected with the USB controller of the communication section 35. The MCU 39 communicates with an MCU 11 of the sensor unit 3.

The sensor unit 3 includes a triaxial acceleration sensor 13, the MCU 11 with the communication function, and a switch section 15. The triaxial acceleration sensor 13 detects accelerations in directions of an Xl axis, a Yl axis, and a Zl axis which are at right angles to one another. The switch section 15 includes switches which are operated by a person. The MCU 11 communicates with the MCU 39 of the wireless communication unit 37 to transmit acceleration data detected by the acceleration sensor 13 and operation information of the switch section 15 to the MCU 39. Then, the acceleration data and the operation information are sent to the CPU 21 via the USB controller 41, the communication section 35, and the chip set 25

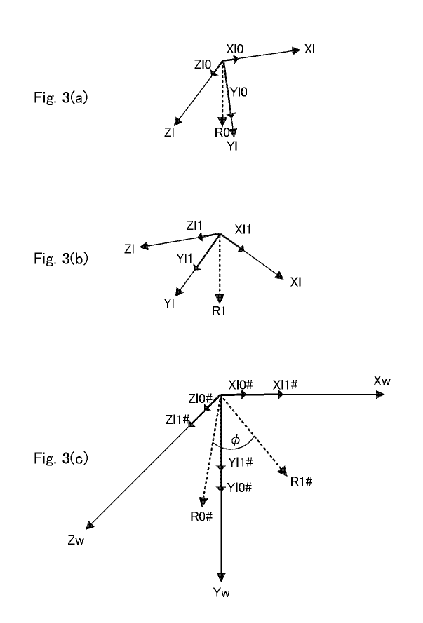

FIGS. 3(a) to 3(c) are explanatory views showing angles which are calculated based on the acceleration data from the sensor unit 3. Incidentally, in the present embodiment, an acceleration caused by the motion of the subject 1 is ignored. In other words, only the gravity acceleration is considered. Referring to FIG. 3(a), it is assumed that the acceleration in the direction of the Xl axis, the acceleration in the direction of the Yl axis, and the acceleration in the direction of the Zl axis from the acceleration sensor 13 are respectively Xl0, Yl0, and Zl0 (the local coordinate system) at the time when the subject 1 starts to perform a predetermined motion (a state of the start time). A resultant vector R0 thereof faces vertically downward, and has the magnitude of the gravity acceleration.

Referring to FIG. 3(b), it is assumed that the acceleration in the direction of the Xl axis, the acceleration in the direction of the Yl axis, and the acceleration in the direction of the Zl axis from the acceleration sensor 13 are respectively Xl1, Yl1, and Zl1 (the local coordinate system) at the time when the subject 1 finishes the predetermined motion (a state of the finish time). A resultant vector R1 thereof faces vertically downward, and has the magnitude of the gravity acceleration.

Referring to FIG. 3(c), acceleration vectors (Xl0#, Yl0#, Zl0#) and (Xl1#, Yl1#, Zl1#) are obtained by converting the acceleration vectors (Xl0, Yl0, Zl0) at the state of the start time and the acceleration vectors (Xl1, Yl1, Zl1) at the state of the finish time into the reference coordinate system. Then, a resultant vector R0# of the acceleration vectors (Xl0#, Yl0#, Zl0#) and a resultant vector R1# of the acceleration vectors (Xl1#, Yl1#, Zl1#) are calculated.

Further then, an angle φ formed by the resultant vectors R0# and R1# is calculated. The angle φ is called a movable range (may be referred to as a movable angle). Incidentally, resultant vectors R (the reference coordinate system) are calculated also with regard to acceleration vectors (Xl#, Yl#, Zl#) from the state of the stat time to the state of the finish time. Then, an angle φ$ formed by the resultant vector R and the resultant vector R0# at the state of the start time is calculated. The angle φ$ is called a state angle.

By the way, FIGS. 3(b) and 3(c) will be used so as to describe another matter. Referring to FIG. 3(b), it is assumed that the acceleration in the direction of the Xl axis, the acceleration in the direction of the Yl axis, and the acceleration in the direction of the Zl axis from the acceleration sensor 13 at one state between the state of the start time and the state of the finish time are respectively Xl1, Yl1, and Zl1 (the local coordinate system). A resultant vector R1 thereof faces vertically downward, and has the magnitude of the gravity acceleration.

Referring to FIG. 3(c), acceleration vectors (Xl1#, Yl1#, Zl1#) are obtained by converting the acceleration vectors (Xl1, Yl1, Zl1) into the reference coordinate system. Then, a resultant vector R1# of the acceleration vectors (Xl1#, Yl1#, Zl1#) is calculated. A vector Rxy to be obtained by projecting the resultant vector R1# onto the XwYw plane and a vector Rzy to be obtained by projecting the resultant vector R1# onto the ZwYw plane are calculated. Then, an angle ω$ formed by the Yw axis and the vector Rxy, and an angle θ$ formed by the Yw axis and the vector Rzy are calculated. The angle ω$ is a displacement angle in a right-left direction, and the angle θ$ is a displacement angle in a front-back direction.

Further, a maximum value ω of the displacement angle ω$ and a maximum value θ of the displacement angle θ$ between the state of the start time and the state of the finish time are calculated. The maximum value ω is called a displacement width (may be referred to as a maximum amplitude) in the front-back direction, and the maximum value θ is called a displacement width (may be referred to as a maximum amplitude) in the right-left direction.

By the way, in the present embodiment, the subject 1 performs a first motion to a sixth motion.

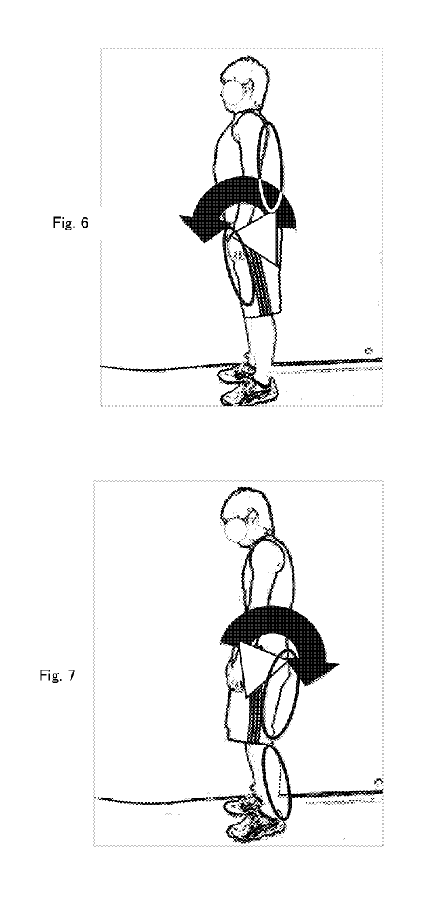

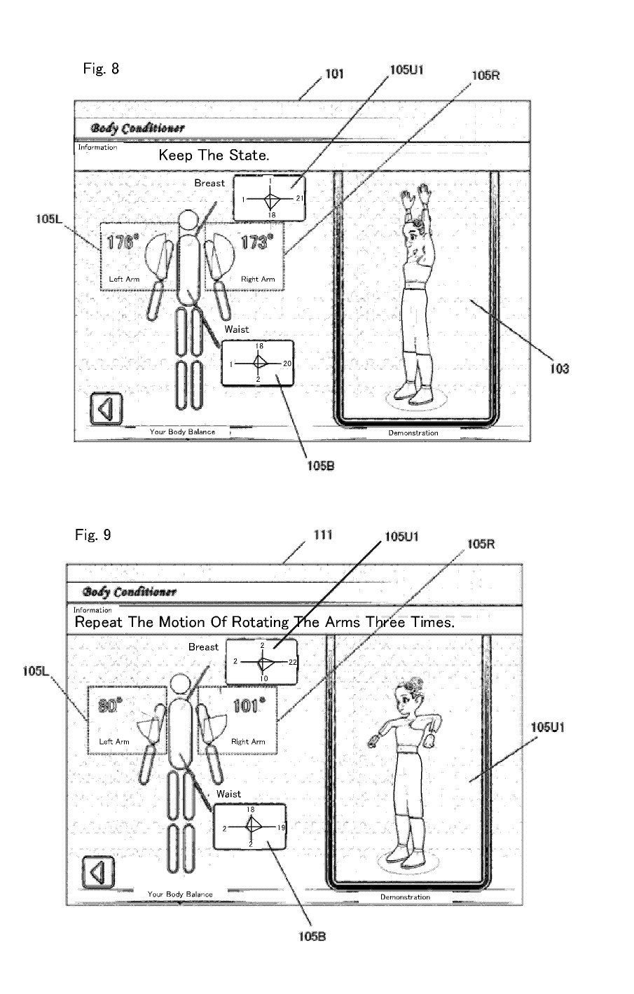

FIG. 8 is an explanatory view showing the first motion (arm raising). Referring to FIG. 8, the PC 7 displays a first motion instruction screen on the monitor 43. The screen contains an instruction section 103. A character which performs the first motion is displayed on the instruction section 103. Accordingly, the subject 1 performs the first motion while imitating the motion of the character. Incidentally, at the start time and at the finish time, that effect is notified the subject 1.

The state of the start time of the first motion is a state where both arms hang down. Then, when the both arms stop by being raised to the utmost limit, its state is the state of the finish time.

Also, the screen contains state angle displaying sections 105L and 105R. The state angle displaying section 105L shows the state angle φ$ based on the acceleration data from the sensor unit 3L in real time by a numeral and an image designating a moving range. The movable range φ of the left arm which is the maximum value of the left state angle φ$ is shown at the state of the finish time. The state angle displaying section 105R shows the state angle φ$ based on the acceleration data from the sensor unit 3R in real time by a numeral and an image designating a moving range. The movable range φ of the right arm which is the maximum value of the right state angle φ$ is shown at the state of the finish time.

Further, the screen contains displacement angle displaying sections 105U1 and 105B. The displacement angle displaying section 105U1 shows the displacement angles ω$ and θ$ based on the acceleration data from the sensor unit 3C in real time by a cobweb chart. The cobweb chart has four vertexes in the front, back, right, and left directions. The right vertex is formed by the maximum value of the displacement angle ω$ of the subject 1 in the right direction. The left vertex is formed by the maximum value of the displacement angle ω$ of the subject 1 in the left direction. The front vertex (the lower vertex on the screen) is formed by the maximum value of the displacement angle θ$ of the subject 1 in the front direction. The back vertex (the upper vertex on the screen) is formed by the maximum value of the displacement angle θ$ of the subject 1 in the back direction. These maximum values are shown also by a numeral. In this case, the maximum value indicates a maximum value at the observation time point in a process where the first motion is performed. Accordingly, the maximum values (the displacement widths) ω and θ in the front, back, right, and left directions in the entire first motion are finally shown at the state of the finish time.

The displacement angle displaying section 105B is only different from the displacement angle displaying section 105U1 in that the displacement angle displaying section 105B is based on the acceleration data from the sensor unit 3W.

FIG. 9 is an explanatory view showing the second motion (forearm rotating). Referring to FIG. 9, the PC 7 displays a second motion instruction screen on the monitor 43. The screen contains the instruction section 103. The character which performs the second motion is displayed on the instruction section 103. Accordingly, the subject 1 performs the second motion while imitating the motion of the character. Incidentally, at the start time and at the finish time, that effect is notified the subject 1.

The state of the start time of the second motion is a state where the upper arms are made horizontal and the forearms hang down. Then, when the forearms stops by being raised to the utmost limit while horizontally keeping the upper arms, its state is the state of the finish time. Such motion is performed three times.

Also, the screen contains the state angle displaying sections 105L and 105R, and the displacement angle displaying sections 105U1 and 105B. These are the same as the corresponding ones in the first motion instruction screen.

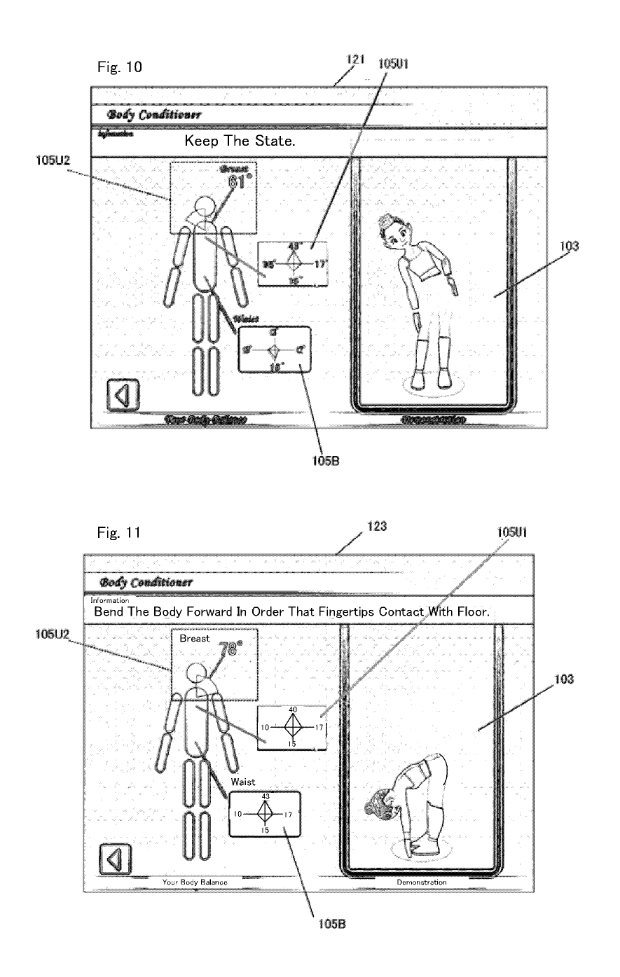

FIG. 10 is an explanatory view showing the third motion (lateral bending). Referring to FIG. 10, the PC 7 displays a third motion instruction screen on the monitor 43. The screen contains the instruction section 103. The character which performs the third motion is displayed on the instruction section 103. Accordingly, the subject 1 performs the third motion while imitating the motion of the character. Incidentally, at the start time and at the finish time, that effect is notified the subject 1.

The state of the start time of the third motion is a state where the both arms hang down while standing erect. Then, when the body stops by being bent to the utmost limit in the left direction, its state is the state of the finish time. Such motion is performed in the right direction.

Also, the screen contains a state angle displaying section 105U2. The state angle displaying section 105U2 shows the state angle φ$ based on the acceleration data from the sensor unit 3C in real time by a numeral and an image designating a moving range. The right movable range φ which is the maximum value of the right state angle φ$ is shown at the state of the finish time of the lateral bending in the right direction. The left movable range φ which is the maximum value of the left state angle φ$ is shown at the state of the finish time of the lateral bending in the left direction.

Further, the screen contains the displacement angle displaying sections 105B and 105U1. The displacement angle displaying sections 105B and 105U1 are the same as the corresponding ones in the first motion instruction screen.

FIG. 11 is an explanatory view showing the fourth motion (forward bending). Referring to FIG. 11, the PC 7 displays a fourth motion instruction screen on the monitor 43. The screen contains the instruction section 103. The character which performs the fourth motion is displayed on the instruction section 103. Accordingly, the subject 1 performs the fourth motion while imitating the motion of the character. Incidentally, at the start time and at the finish time, that effect is notified the subject 1.

The state of the start time of the fourth motion is a state where the both arms hang down while standing erect. Then, when the body stops by being bent to the utmost limit in the front direction, its state is the state of the finish time.

Also, the screen contains the state angle displaying section 105U2, and the displacement angle displaying sections 105B and 105U1. These are the same as the corresponding ones in the third motion instruction screen.

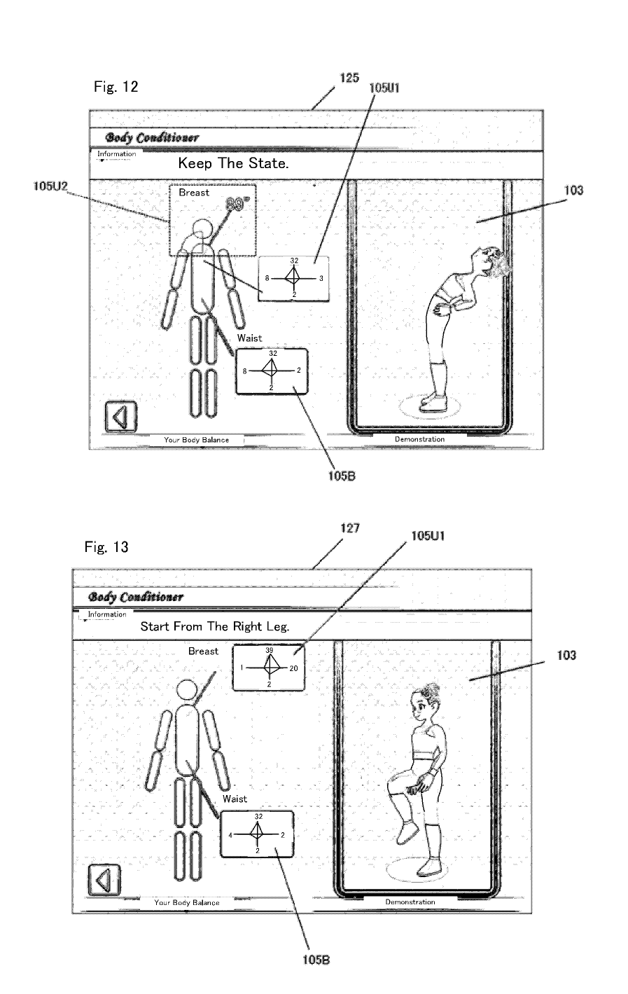

FIG. 12 is an explanatory view showing the fifth motion (backward bending). Referring to FIG. 12, the PC 7 displays a fifth motion instruction screen on the monitor 43. The screen contains the instruction section 103. The character which performs the fifth motion is displayed on the instruction section 103. Accordingly, the subject 1 performs the fifth motion while imitating the motion of the character. Incidentally, at the start time and at the finish time, that effect is notified the subject 1.

The state of the start time of the fifth motion is a state where the both arms hang down while standing erect. Then, when the body stops by being bent to the utmost limit in the back direction, its state is the state of the finish time.

Also, the screen contains the state angle displaying section 105U2, and the displacement angle displaying sections 105B and 105U1. These are the same as the corresponding ones in the third motion instruction screen.

FIG. 13 is an explanatory view showing the sixth motion (thigh raising). Referring to FIG. 13, the PC 7 displays a sixth motion instruction screen on the monitor 43. The screen contains the instruction section 103. The character which performs the sixth motion is displayed on the instruction section 103. Accordingly, the subject 1 performs the sixth motion while imitating the motion of the character. Incidentally, at the start time and at the finish time, that effect is notified the subject 1.

The state of the start time of the sixth motion is a state where the both arms hang down while standing erect. Then, when the left knee is raised until the thigh becomes horizontal and then is put down, its state is the state of the finish time regarding the left. Such motion is performed also by the right leg. When one set consists of the right and left, the three sets are performed.

Also, the screen contains the displacement angle displaying sections 105U1 and 105B. The displacement angle displaying sections 105U1 and 105B are the same as the corresponding ones in the first motion instruction screen.

By the way, the movable range φ of the right arm and the movable range φ of the left arm in the first motion (arm raising) are respectively referred to as AR and AL. The displacement width θ in the front direction in the fourth motion (forward bending) is referred to as CF. The displacement width ω of the right side and the displacement width ω of the left side in the sixth motion (thigh raising) are respectively referred to as WR and WL. In that case, as shown in the following Table 1, posture can be classified into eight patterns.

TABLE 1

| Pattern | | | |
|---|---|---|---|
| Title | Shoulder | Load | Pelvis |
| A-[1] | Left Ascent | Right | Forward Tilt |
| | (AL > AR) | (WL > WR) | (CF > 60) |
| A-[2] | Left Ascent | Right | Backward Tilt |
| | (AL > AR) | (WL > WR) | (CF ≦ 60) |
| B-[1] | Right Ascent | Left | Forward Tilt |
| | (AL < AR) | (WL < WR) | (CF > 60) |
| B-[2] | Right Ascent | Left | Backward Tilt |
| | (AL < AR) | (WL < WR) | (CF ≦ 60) |
| C-[1] | Right Ascent | Right | Forward Tilt |
| | (AL < AR) | (WL > WR) | (CF > 60) |
| C-[2] | Right Ascent | Right | Backward Tilt |
| | (AL < AR) | (WL > WR) | (CF ≦ 60) |
| D-[1] | Left Ascent | Left | Forward Tilt |
| | (AL > AR) | (WL < WR) | (CF > 60) |
| D-[2] | Left Ascent | Left | Backward Tilt |
| | (AL > AR) | (WL < WR) | (CF ≦ 60) |

In this case, the movable range AR of the right arm is calculated on the basis of the output of the sensor unit 3R of the right arm. The movable range AL of the left arm is calculated on the basis of the output of the sensor unit 3L of the left arm. The displacement widths CF, WR, and WL are calculated on the basis of the output of the sensor unit 3W.

(Pattern A-[1]) FIG. 4(a) (forward-looking), FIG. 4(b) (backward-looking), and FIG. 6 show the posture when determining the pattern A-[1].

Referring to Table 1, the movable range AL of the left arm is greater than the movable range AR of the right arm. This indicates that the left shoulder is higher than the right shoulder (a left shoulder ascent, and a right shoulder descent). The displacement width WL of the left side is greater than the displacement width WR of the right side. This indicates that a right side of a pelvis is higher than a left side thereof (a right waist ascent), and the pelvis is displaced (is shifted) rightward (a right load). Also, these indicate that a head inclines leftward. Further, the displacement width CF is greater than 60 degrees. This indicates that the pelvis inclines forward, and whereby the lower back warps (the back warps backward). That is, the chest is thrown out and the lower back warps, and the knees extend completely. In this posture, the body weight is apt to be applied to the tiptoes.

(Pattern A-[2]) FIG. 4(a) (forward-looking), FIG. 4(b) (backward-looking), and FIG. 7 show the posture when determining the pattern A-[2].

Referring to Table 1, the movable range AL of the left arm is greater than the movable range AR of the right arm. The displacement width WL of the left side is greater than the displacement width WR of the right side. These are the same as Pattern A-[1].

The displacement width CF is 60 degrees or below. This indicates that the pelvis inclines backward, and the back is the stoop. That is, the back is rounded, and the knees are slightly apt to bend. In this posture, the body weight is apt to be applied to the heels.

(Pattern B-[1]) This pattern is obtained by reversing the right and left of Pattern A-[1].

(Pattern B-[2]) This pattern is obtained by reversing the right and left of Pattern A-[2].

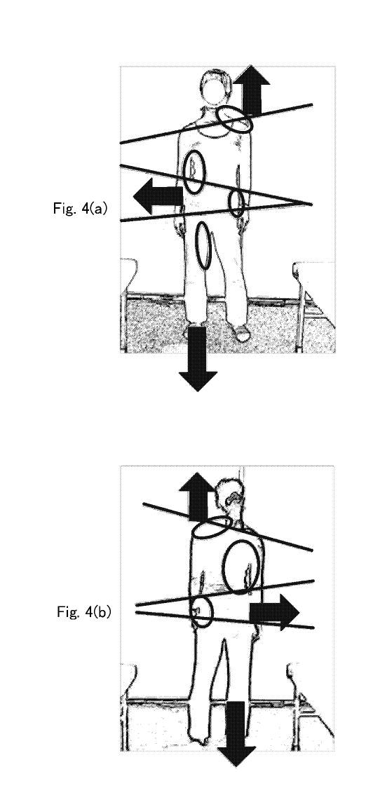

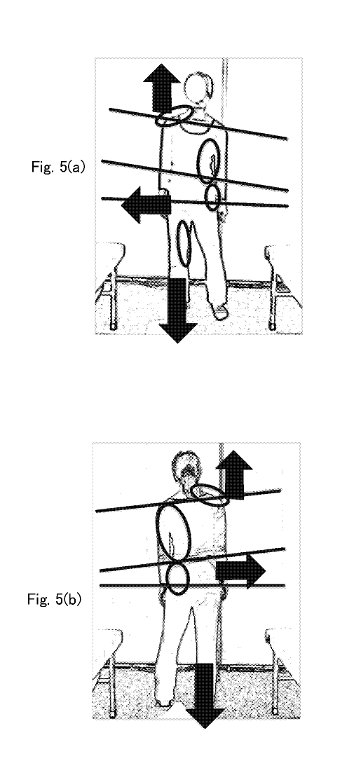

(Pattern C-[1]) FIG. 5(a) (forward-looking), FIG. 5(b) (backward-looking), and FIG. 6 show the posture when determining the pattern C-[1].

Referring to Table 1, the movable range AR of the right arm is greater than the movable range AL of the left arm. This indicates that the right shoulder is higher than the left shoulder (a right shoulder ascent, and a left shoulder descent). The displacement width WL of the left side is greater than the displacement width WR of the right side. This indicates that a right side of a pelvis is higher than a left side thereof (a right waist ascent), and the pelvis is displaced (is shifted) rightward (a right load). Also, these indicate that a head inclines leftward. Further, the displacement width CF is greater than 60 degrees. This indicates that the pelvis inclines forward, and whereby the lower back warps (the back warps backward). That is, the chest is thrown out and the lower back warps, and the knees extend completely. In this posture, the body weight is apt to be applied to the tiptoes.

(Pattern C-[2]) FIG. 5(a) (forward-looking), FIG. 5(b) (backward-looking), and FIG. 7 show the posture when determining the pattern C-[2].

Referring to Table 1, the movable range AR of the right arm is greater than the movable range AL of the left arm. The displacement width WL of the left side is greater than the displacement width WR of the right side. These are the same as Pattern C-[1].

The displacement width CF is 60 degrees or below. This indicates that the pelvis inclines backward, and the back is the stoop. That is, the back is rounded, and the knees are slightly apt to bend. In this posture, the body weight is apt to be applied to the heels.

(Pattern D-[1]) This pattern is obtained by reversing the right and left of Pattern C-[1].

(Pattern D-[2]) This pattern is obtained by reversing the right and left of Pattern C-[2].

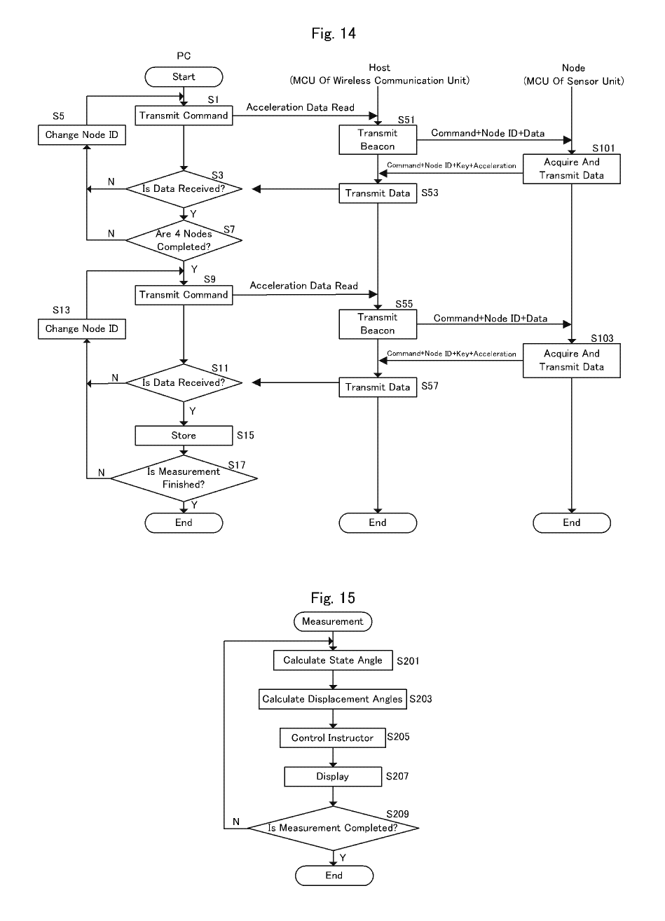

FIG. 14 is a view showing the communication procedure among the PC 7, the MCU 39 (hereinafter referred to as a “host 39” in the explanation of this figure) of the wireless communication unit 37, and the MCU 11 (hereinafter referred to as a “node 11” in the explanation of this figure) of the sensor unit 3. Referring to FIG. 14, in step S1, the PC 7 sends a read command of acceleration data, a node ID, and data to the host 39. Then, in step S51, the host 39 transmits a beacon including the read command, the node ID, and the data to the node 11. In this case, the node ID is information for identifying the node 11, i.e., the sensor unit 3. In the present embodiment, the different node IDs are respectively assigned to the four sensor units 3.

When the node 11 receives the beacon including the node ID assigned to itself, in step S101, the node 11 transmits the command as received from the host 39, its own node ID, and acceleration data (Xl, Yl, Zl) as acquired from the acceleration sensor 13 to the host 39.

In step S53, the host 39 transmits the data as received from the node 11 to the PC 7. In step S3, the PC 7 determines whether or not the data from the host 39 is received, the process proceeds to step S5 if the data is not received, conversely the process proceeds to step S7 if the data is received. In step S5, the PC 7 changes the node ID which is included in the beacon, and then proceeds to step S1. Because it needs to detect all of the four nodes 11.

In step S7, the PC 7 determines whether or not all of the four nodes 11 (sensor units 3) are detected, the process proceeds to step S5 if all are not detected, conversely the process proceeds to step S9 if all are detected. Because all of the four nodes 11 are used so as to measure the posture.

In step S9, the PC 7 sends a read command of acceleration data, a node ID, and data to the host 39. Then, in step S55, the host 39 transmits the beacon including the read command, the node ID, and the data to the node 11. In step S103, the node 11 transmits the command as received from the host 39, its own node ID, and the acceleration data (Xl, Yl, Zl) of the acceleration sensor 13 to the host 39.

In step S57, the host 39 transmits the data as received from the node 11 to the PC 7. In step S11, the PC 7 determines whether or not the data from the host 39 is received, the process proceeds to step S13 if the data is not received, conversely the process proceeds to step S15 if the data is received. In step S13, the PC 7 changes the node ID which is included in the beacon, and then proceeds to step S9. On the other hand, in step S15, the PC 7 stores the received data in the main memory 23 or the HDD 31. In step S17, the PC 7 determines whether or not the measurement of the posture is finished, i.e., the measurement is completed until the sixth motion, the process proceeds to step S13 if it is not completed, otherwise the process is ended.

FIG. 15 is a flow chart showing a measuring process by the PC 7. Referring to FIG. 15, in step S201, the PC 7 calculates the state angle φ$ on the basis of the acceleration data from the sensor unit 3. In step S203, the PC 7 calculates the displacement angles ω$ and θ$ on the basis of the acceleration data from the sensor unit 3. In step S205, the PC 7 controls animation of the character (representing an instructor) in the instruction section 103. In step S207, the PC 7 displays the motion instruction screen (FIGS. 8 to 13) in accordance with the results of the steps S201 to S205. In step S209, the PC 7 determines whether or not the measurement of one motion is completed, the process proceeds to step S201 if it is not completed, otherwise the process for the one motion is ended. The measuring process of FIG. 15 is performed for each of the first to sixth motions.

Incidentally, the state angle φ$ at the time point when “YES” is determined in step S209 corresponds to the movable range φ in the motion. Also, the maximum value of the displacement angle ω$ until the time point when “YES” is determined in step S209 corresponds to the displacement width ω in the motion, and the maximum value of the displacement angle θ$ until the time point when “YES” is determined in step S209 corresponds to the displacement width θ in the motion.

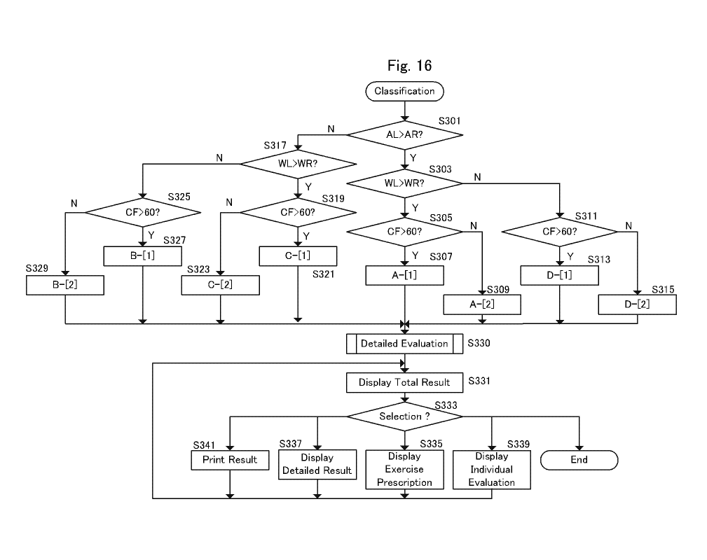

FIG. 16 is a flow chart showing a classifying process by the PC 7. Referring to FIG. 16, in step S301, the PC 7 determines whether or not the movable range AL is greater than the movable range AR, the process proceeds to step S303 if it is greater, otherwise the process proceeds to step S317. In step S303, the PC 7 determines whether or not the displacement width WL is greater than the displacement width WR, the process proceeds to step S305 if it is greater, otherwise the process proceeds to step S311. In step S305, the PC 7 determines whether or not the displacement width CF exceeds 60 degrees, the process proceeds to step S307 if it exceeds, otherwise the process proceeds to step S309. In step S307, the PC 7 classifies the posture pattern of the subject 1 into Pattern A-[1]. On the other hand, in step S309, the PC 7 classifies the posture pattern of the subject 1 into Pattern A-[2].

In step S311 after “NO” is determined in step S303, the PC 7 determines whether or not the displacement width CF exceeds 60 degrees, the process proceeds to step S313 if it exceeds, otherwise the process proceeds to step S315. In step S313, the PC 7 classifies the posture pattern of the subject 1 into Pattern D-[1]. On the other hand, in step S315, the PC 7 classifies the posture pattern of the subject 1 into Pattern D-[2].

In step S317 after “NO” is determined in step S301, the PC 7 determines whether or not the displacement width WL is greater than the displacement width WR, the process proceeds to step S319 if it is greater, otherwise the process proceeds to step S325. In step S319, the PC 7 determines whether or not the displacement width CF exceeds 60 degrees, the process proceeds to step S321 if it exceeds, otherwise the process proceeds to step S323. In step S321, the PC 7 classifies the posture pattern of the subject 1 into Pattern C-[1]. On the other hand, in step S323, the PC 7 classifies the posture pattern of the subject 1 into Pattern C-[2].

In step S325 after “NO” is determined in step S317, the PC 7 determines whether or not the displacement width CF exceeds 60 degrees, the process proceeds to step S327 if it exceeds, otherwise the process proceeds to step S329. In step S327, the PC 7 classifies the posture pattern of the subject 1 into Pattern B-[1]. On the other hand, in step S329, the PC 7 classifies the posture pattern of the subject 1 into Pattern B-[2].

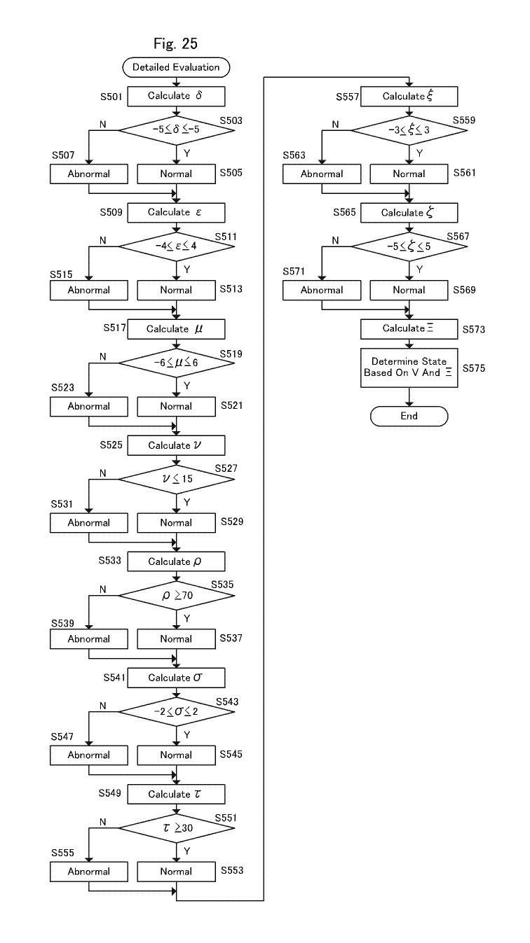

In step S330, the PC 7 performs a detailed evaluation process as shown in FIG. 25 as described below. In step S331, the PC 7 determines a human body image in accordance with the posture pattern of the subject 1 (S307, S309, S327, S329, S321, S323, S313, or S315), and then displays a total result screen on the monitor 43.

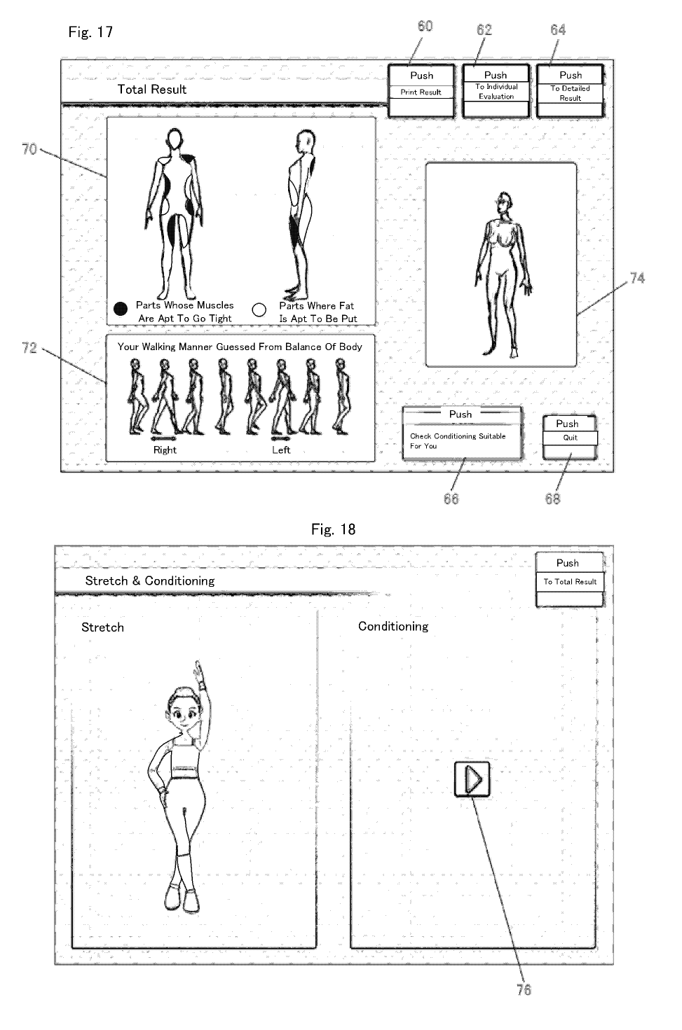

FIG. 17 is a view showing an example of the total result screen. Referring to FIG. 17, the total result screen is an example corresponding to the posture pattern A-[1]. The total result screen contains buttons 60, 62, 64, 66, and 68, a first frame 70, a second frame 72, and a third frame 74.

The human body image representing the posture pattern A-[1] is displayed in the first frame 70. In the human body image, regions whose muscles are apt to go tight are indicated by a first color (black color in the figure), and regions on which fat is apt to be put are indicated by a second color (white color regions surrounded by the black line in the figure). Also, the human body image can is rotated, enlarged, or reduced by operating the keyboard 32 or the mouse 34. Or, the human body image may rotate automatically.

A walking manner as guessed on the basis of the posture pattern A-[1] is shown in the second frame 72 by animation or static images.

A human body image representing the posture pattern A-[1], on which excess fat is put on, is displayed in the third frame 74. In this case, the excess fat is put on the regions indicated by the second color in the first frame 70. Also, the human body image can is rotated, enlarged, or reduced by operating the keyboard 32 or the mouse 34. Or, the human body image may rotate automatically.

Returning to FIG. 16, in step S333, the PC 7 determines which of the buttons 60 to 68 displayed on the total result screen is selected, and proceeds to the next step in accordance with the result of the selection. That is, the PC 7 proceeds to step S335 if the exercise prescription button 66 on the total result screen is selected, proceeds to step S337 if the detailed result button 64 on the total result screen is selected, proceeds to step S339 if the individual evaluation button 62 on the total result screen is selected, proceeds to step S341 if the printing button 60 on the total result screen is selected, and ends the process if the quitting button 68 on the total result screen is selected.

In step S335, the PC 7 displays an exercise prescription screen on the monitor 43 in accordance with the posture pattern of the subject 1. The exercise prescription screen has a left area and a right area which have buttons 76 respectively. As shown in FIG. 18, when the button 76 in the left area is selected, animation of a character is displayed on the left area. The exercise for correcting the posture of the subject 1 is hereby instructed by the animation of the character in accordance with the posture pattern. In the example of FIG. 18, the posture is corrected by a stretching exercise. Incidentally, FIG. 18 corresponds to the posture pattern A-[1].

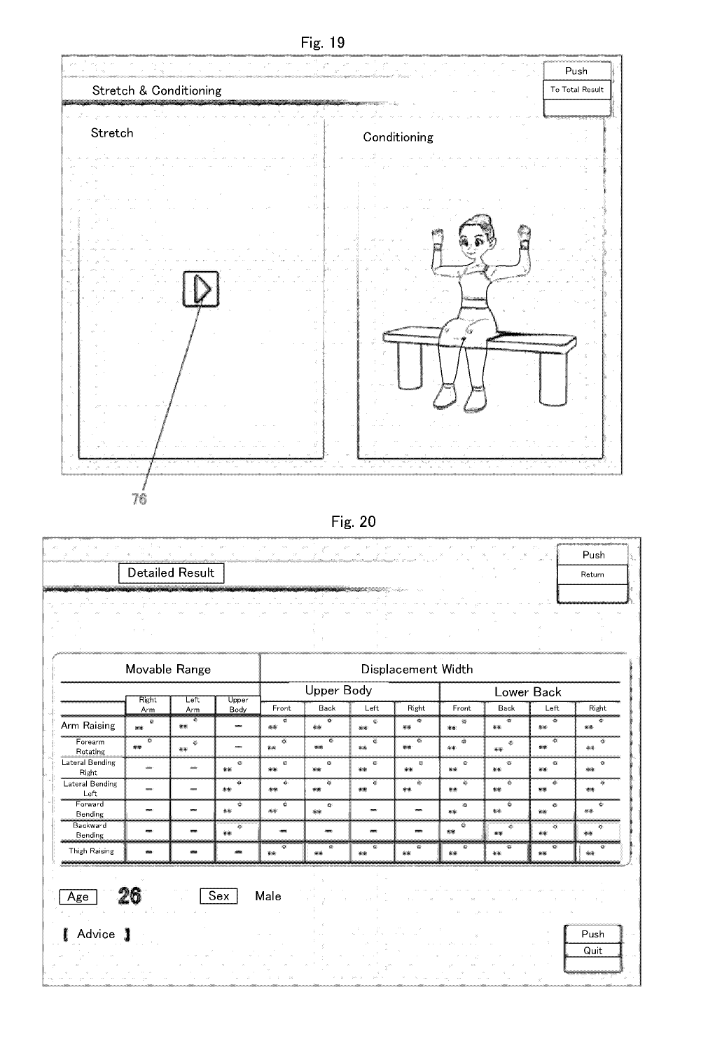

On the other hand, as shown in FIG. 19, when the button 76 in the right area is selected, animation of a character is displayed on the right area. The exercise for correcting the posture of the subject 1 is hereby instructed by the animation of the character in accordance with the posture pattern. In the example of FIG. 19, the posture is corrected by a conditioning exercise. Incidentally, FIG. 19 corresponds to the posture pattern A-[1].

For example, in the stretching exercise for Pattern A-[1], the right leg is crossed from the back of the left leg. The left hand is placed on the hip, and the right hand is raised. The body is slowly inclined leftward while exhaling breath. The state is held for 30 seconds at the reasonable position, and then is slowly turned back. These are represented by the animation of the character. For example, in the conditioning exercise for Pattern A-[1], the right fist is raised with the elbow flexed to 90 degrees. The body is bent so that the right elbow is in contact with the left knee while exhaling breath. In this case, it is conscious of the belly. The state is slowly turned back. Two sets each of which consists of 10 times are performed. These are represented by the animation of the character.

For example, the stretching exercise for Pattern A-[2] includes sitting flat with the left leg extended. Then, the body is bent forward while throwing out the chest and exhaling breath. The state is held for 30 seconds at the reasonable position, and then is slowly turned back. These are represented by the animation of the character. For example, in the conditioning exercise for Pattern A-[2], the arms are opened outward with the elbows flexed to 90 degrees. The left arm is pulled backward while exhaling breath and thrusting the right fist upward. It is conscious that the body is twisted leftward. The state is turned back if the breath is completely exhaled. Two sets each of which consists of 10 times are performed. These are represented by the animation of the character.

For example, in the stretching exercise for Pattern B-[1], the legs are opened backward and forward with the front right leg, and the left ankle is held by the left hand. Then, the left foot is moved toward the hip to extend the foreside of the thigh while exhaling breath. The state is held for 30 seconds at the reasonable position, and then is slowly turned back. For example, in the conditioning exercise for Pattern B-[1], the left fist is raised with the elbow flexed to 90 degrees. Then, the body is bent so that the left elbow is in contact with the right knee while exhaling breath. In this case, it is conscious of the belly. Then, the state is slowly turned back. Two sets each of which consists of 10 times are performed. These are represented by the animation of the character.

For example, in the stretching exercise for Pattern B-[2], the left hand is kissingly put on the head. Then, the head is slowly inclined leftward while exhaling breath. The state is held for 30 seconds at the reasonable position, and then is slowly turned back. For example, in the conditioning exercise for Pattern B-[2], the arms are opened outward with the elbows flexed to 90 degrees. Then, the right arm is pulled backward while exhaling breath and thrusting the left fist upward. It is conscious that the body is twisted rightward. Two sets each of which consists of 10 times are performed. These are represented by the animation of the character.

For example, in the stretching exercise for Pattern C-[1], the legs are opened backward and forward with the front right leg, and the left ankle is held by the left hand. Then, the left foot is moved toward the hip to extend the foreside of the thigh while exhaling breath. The state is held for 30 seconds at the reasonable position, and then is slowly turned back. These are represented by the animation of the character. For example, in the conditioning exercise for Pattern C-[1], the arms are tightly put on the sides, and the elbows are flexed to 90 degrees. The left fist is thrust forward while the right arm is pulled backward, exhaling breath. The state is turned back if the breath is completely exhaled. Two sets each of which consists of 10 times are performed. These are represented by the animation of the character.

For example, the stretching exercise for Pattern C-[2] includes sitting flat with the right leg extended. Then, the body is bent forward while throwing out the chest and exhaling breath. The state is held for 30 seconds at the reasonable position, and then is slowly turned back. These are represented by the animation of the character. For example, in the conditioning exercise for Pattern C-[2], both the hands are put on the back of the head. The body is slowly inclined rightward while exhaling breath. It is conscious of the right flank. The state is turned back if the breath is completely exhaled. Two sets each of which consists of 10 times are performed. These are represented by the animation of the character.

For example, in the stretching exercise for Pattern D-[1], the right leg is crossed from the back of the left leg. Then, the left hand is placed on the hip, and the right hand is raised. The body is slowly inclined leftward while exhaling breath. The state is held for 30 seconds at the reasonable position, and then is slowly turned back. These are represented by the animation of the character. For example, in the conditioning exercise for Pattern D-[1], the arms are tightly put on the sides, and the elbows are flexed to 90 degrees. Then the right fist is thrust forward while the left arm is pulled backward, exhaling breath. The state is turned back if the breath is completely exhaled. Two sets each of which consists of 10 times are performed. These are represented by the animation of the character.

For example, in the stretching exercise for Pattern D-[2], the right hand is kissingly put on the head. Then, the head is slowly inclined rightward while exhaling breath. The state is held for 30 seconds at the reasonable position, and then is slowly turned back. These are represented by the animation of the character. For example, in the conditioning exercise for Pattern D-[2], both the hands are put on the back of the head. The body is slowly inclined leftward while exhaling breath. It is conscious of the left flank. The state is turned back if the breath is completely exhaled. Two sets each of which consists of 10 times are performed. These are represented by the animation of the character.

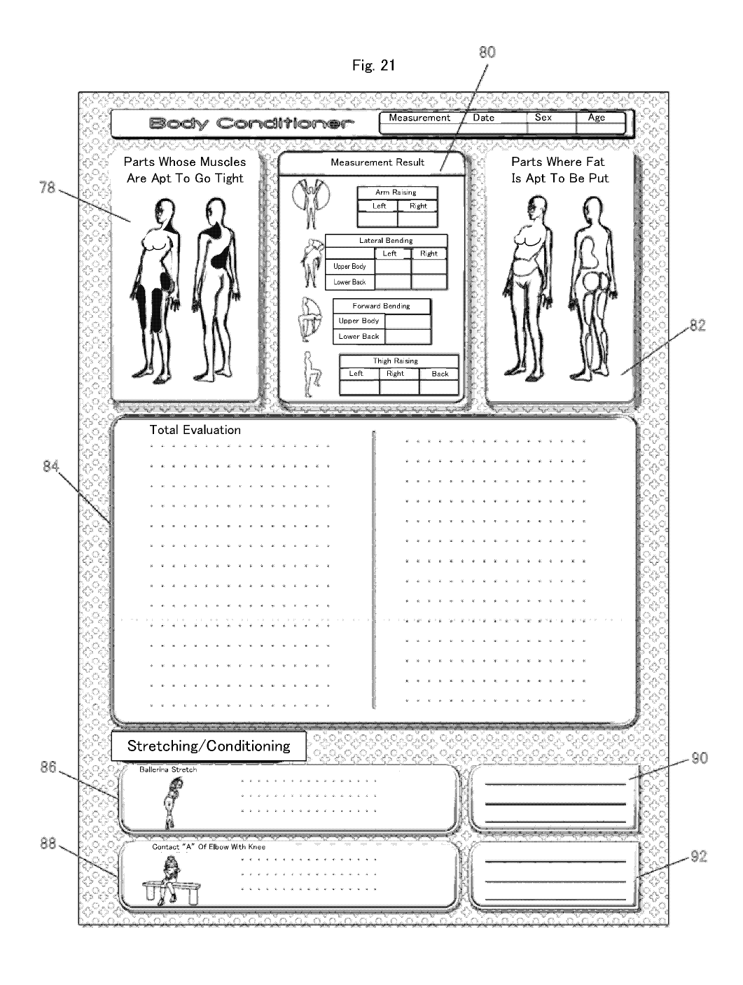

Returning to FIG. 16, in step S337, the PC 7 displays a detailed result screen of FIG. 20 on the monitor 43. Also, in step S339, the PC 7 displays an individual evaluation screen on the monitor 43. The individual evaluation screen includes the evaluation for each of the first to sixth motions. Also, in step S341, the PC 7 prints a picture which contains the human body image of the total result in step S331 and the detailed evaluation in step S330 (see FIG. 21).

FIG. 21 is a view showing an example of the printed result screen in step S341. Incidentally, FIG. 21 corresponds to the posture pattern A-[1].

Referring to FIG. 21, the printed result contains a first frame 78, a second frame 80, a third frame 82, a fourth frame 84, a fifth frame 86, a sixth frame 88, a seventh frame 90, and a eighth frame 92.

The human body image corresponding to the posture pattern is displayed in the first frame 78. In the human body image, the regions whose muscles are apt to go tight are indicated by the first color (black color in the figure). The predetermined ones of the measurement results displayed on the total result screen of FIG. 20 are displayed in the second frame 80. The human body image corresponding to the posture pattern is displayed in the third frame 82, and the regions on which fat is apt to be put are indicated by the second color (the white color regions surrounded by the black line in the figure).

The explanation of the stretching exercise corresponding to the exercise prescription screen (see FIG. 18) is displayed in accordance with the posture pattern in the fifth frame 86. The explanation of the current state is displayed in the seventh frame 90 together with the explanation of the state after continuously performing the stretching exercise of the fifth frame 86 (before-after analysis).

The explanation of the conditioning exercise corresponding to the exercise prescription screen (see FIG. 19) is displayed in accordance with the posture pattern in the sixth frame 88. The explanation of the current state is displayed in the eighth frame 92 together with the explanation of the state after continuously performing the conditioning exercise of the sixth frame 88 (before-after analysis).

The detailed evaluation (hereinafter referred to as a “detail evaluation”) is displayed in the fourth frame 84. In what follows, the evaluation method will be described.

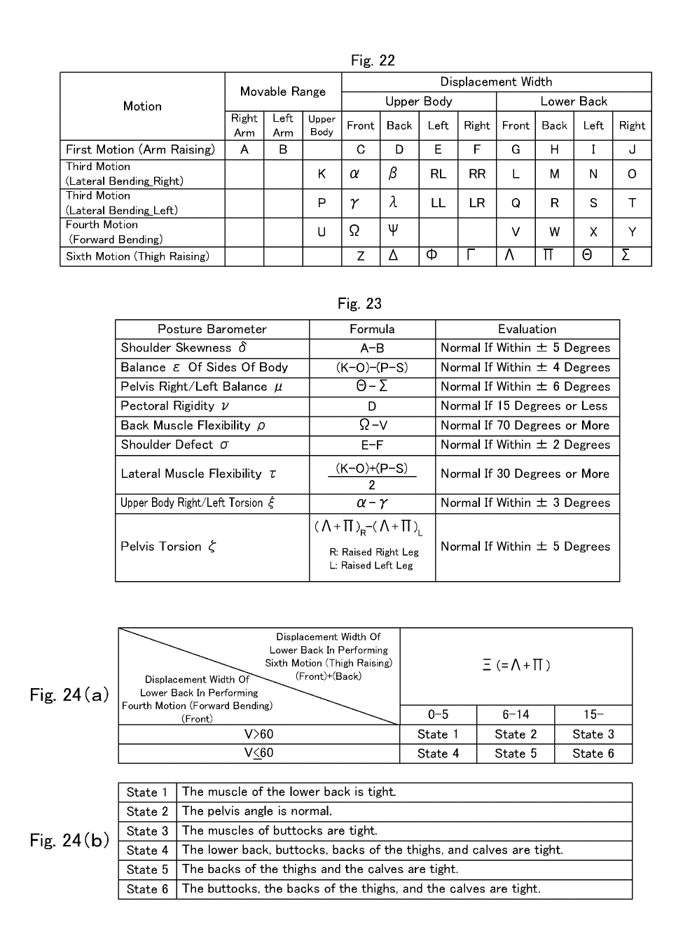

FIG. 22 is a view showing the table of parameters which are used for the detail evaluation. Referring to FIG. 22, the movable ranges and the displacement widths based on the sensor units 3R, 3L, 3C, and 3W in the first motion, the third motion, the fourth motion, and the sixth motion are used so as to perform the detail evaluation.

The particularity is as follows with regard to the first motion (arm raising). The movable range A of the right arm based on the sensor unit 3R, the movable range B of the left arm based on the sensor unit 3L, the displacement widths C, D, F, and E of the upper body in the front, back, right, and left directions based on the sensor unit 3C, and the displacement widths G, H, J, and I of the lumbar part in the front, back, right, and left directions based on the sensor unit 3W are used.

The particularity is as follows with regard to the rightward lateral bending in the third motion (lateral bending). The movable range K of the upper body based on the sensor unit 3C, the displacement widths α, β, RR, and RL of the upper body in the front, back, right, and left directions based on the sensor unit 3C, and the displacement widths L, M, O, and N of the lumbar part in the front, back, right, and left directions based on the sensor unit 3W are used.

The particularity is as follows with regard to the leftward lateral bending in the third motion (lateral bending). The movable range P of the upper body based on the sensor unit 3C, the displacement widths γ, λ, LR, and LL of the upper body in the front, back, right, and left directions based on the sensor unit 3C, and the displacement widths Q, R, T, and S of the lumbar part in the front, back, right, and left directions based on the sensor unit 3W are used.

The particularity is as follows with regard to the fourth motion (forward bending). The movable range U of the upper body based on the sensor unit 3C, the displacement widths Ω and ψ of the upper body in the front and back directions based on the sensor unit 3C, and the displacement widths [symbol] , W, Y, and X of the lumbar part in the front, back, right, and left directions based on the sensor unit 3W are used.

The particularity is as follows with regard to the sixth motion (thigh raising). The displacement widths Z, Δ, Γ, and φ of the upper body in the front, back, right, and left directions based on the sensor unit 3C, and the displacement widths [symbol] , [symbol] , Σ, and Θ of the lumbar part in the front, back, right, and left directions based on the sensor unit 3W are used.

FIG. 23 is an explanatory view showing posture barometers for the detail evaluation. Referring to FIG. 23, the posture barometers includes shoulder skewness δ, balance ε of sides of a body (lateral muscle balance), pelvis right/left balance μ, pectoral rigidity v, back muscle flexibility ρ, a shoulder defect σ, lateral muscle flexibility τ, upper body right/left torsion ξ, and pelvis torsion ζ. These are expressed by the following formulae using the parameters of FIGS. 22.

δ=A−B

ε=(K−O)−(P−S)

μ=Θ−Σ

v=D

ρ=Ω−

[symbol]

σ=E−F

τ=((K−O)+(P−S))/2

ξ=α-γ

ζ=( [symbol] + [symbol] )_{R}−( [symbol] + [symbol] )_{L}

The shoulder skewness δ is expressed by a difference (A−B) between the movable range A of the right arm and the movable range B of the left arm when the upper arms are raised. The arm corresponding to the daily-lowering shoulder is hard to be raised, and therefore the shoulder skewness δ can be expressed by the difference between them.

The balance ε of sides of a body is a difference ((K−O)−(P−S)) between a value (K−O) obtained by subtracting the pelvis angle (the displacement width of the lumbar part in the right direction) O from the movable range K of the upper body when the upper body is bent rightward, and a value (P−S) obtained by subtracting the pelvis angle (the displacement width of the lumbar part in the left direction) S from the movable range P of the upper body when the upper body is bent leftward, and represents the balance of the right/left flexibility of the lateral muscles of the upper body. When it is tried to bend the upper body, if the lateral muscle does not have the flexibility, the upper body is bent by moving the pelvis (vicarious movement), and therefore the pelvis angle, i.e., the vicarious movement is subtracted from the movable range of the upper body so as to obtain the real bend of the upper body by the lateral muscle.

The pelvis right/left balance μ is a difference between the right and left tilts of the pelvis when performing the thigh raising, i.e., a difference (Θ−Σ) between the displacement width Θ of the lumbar part in the left direction and the displacement width Σ of the lumbar part in the right direction when performing the thigh raising. In the case where there are a difference between the right muscular strength and the left muscular strength around the waist, displacement of the pelvis, and so on, the absolute value of the value μ becomes great. The difference between the right muscular strength and the left muscular strength around the waist, and the displacement of the pelvis cause the motion of raising the leg using the lumbar part, i.e., the vicarious movement. Accordingly, it is possible to recognize and evaluate the right/left balance of the pelvis by the difference between the right vicarious movement Σ and the left vicarious movements Θ accompanying the right/left thigh raising.

The pectoral rigidity v is a displacement width D of the upper body in the back direction when the upper arms are raised. In the case where the pectoral is tight, the arms are hard to be opened rightward and leftward, and therefore the upper body is apt to be warped backward. As the result, the value of the rigidity v becomes great. Also, in the case where the pectoral is tight and therefore the rigidity v is great, the case causes the stoop. If the pectoral is tight, it encumbers the motion of raising the upper arms. The relatively-great vicarious movement is therefore performed so as to raise the upper arms. Thus, the rigidity of the pectoral can be evaluated by the extent of the displacement width D representing the vicarious movement.

The back muscle flexibility ρ is a value (Ω− [symbol] ) obtained by subtracting a forward-tilt angle [symbol] of the pelvis (the displacement width V of the lumbar part in the front direction) from a forward-tilt angle Ω of the upper body (the displacement width Ω of the upper body in the front direction) when performing the forward bending. In this way, the real forward bending by the muscle of the back is obtained by eliminating the bending by the pelvis. The value ρ hereby represents the flexibility of the back muscle.

The shoulder defect σ is an angular difference of the upper body when the upper arms are raised, i.e., a difference (E−F) between the displacement width E of the upper body in the left direction and the displacement width F of the upper body in the right direction when the upper arms are raised. In the case where any one of the shoulders has pain and so on when the upper arms are raised, the assist is often performed by inclining the upper body, i.e., raising the shoulder having the pain and so on (the vicarious movement). As the result, the defect of the shoulder can be represented by the value σ which is the difference between the right vicarious movement F and the left vicarious movement E.

The lateral muscle flexibility τ is an average value of a value (K−O) obtained by subtracting the pelvis angle (the displacement width of the lumbar part in the right direction) O from the movable range K of the upper body when performing the lateral bending rightward and a value (P−S) obtained by subtracting the pelvis angle (the displacement width of the lumbar part in the left direction) S from the movable range P of the upper body when performing the lateral bending leftward. The reason for subtracting the pelvis angle from the movable range of the upper body is the same as that of the balance ε of sides of a body.

The upper body right/left torsion ξ is a difference (α−γ) between the displacement width α of the upper body in the front direction when performing the lateral bending rightward, and the displacement width γ of the upper body in the front direction when performing the lateral bending leftward. If there is the torsion of the upper body, when the lateral bending is performed to the torsional side, the upper body is apt to incline forward. Therefore, the right/left torsion of the upper body can be expressed by the value ξ.

The pelvis torsion ζ is a difference (( [symbol] + [symbol] )_{R}−( [symbol] + [symbol] )_{L}) between a sum ( [symbol] + [symbol] )_{R} of the displacement width [symbol] of the lumbar part in the front direction when performing the right thigh raising and the displacement width [symbol] of the lumbar part in the back direction when performing the right thigh raising, and a sum ( [symbol] + [symbol] )_{L} of the displacement width [symbol] of the lumbar part in the front direction when performing the left thigh raising and the displacement width [symbol] of the lumbar part in the back direction when performing the left thigh raising. The values ( [symbol] + [symbol] )_{R} and ( [symbol] + [symbol] )_{L} respectively represent the backward-tilt angle of the pelvis when the right leg is raised, and the backward-tilt angle of the pelvis when the left leg is raised. In this case, since the backward-tilt angle is a backward-tilt angle relative to the ordinary condition of the subject, the value [symbol] is added to the value [symbol] . The backward-tilt angles are unsymmetrical if the balance of the muscle around the pelvis is bad, and therefore the absolute value of the value ζ i.e., the torsion of the pelvis becomes great.

Also, when the muscle to be originally used (the principal muscle) in raising the leg is in bad condition or in bad shape, a human tries to raise the leg using the lumber part (the vicarious movement). Accordingly, if the right leg raising is assist by the vicarious movement, the backward-tilt angle ( [symbol] + [symbol] )_{R} of the pelvis, which represents the vicarious movement, becomes great in performing the right leg raising. On the other hand, if the left leg raising is assist by the vicarious movement, the backward-tilt angle ( [symbol] + [symbol] )_{L} of the pelvis, which represents the vicarious movement, becomes great in performing the left leg raising. Thus, if the absolute value of the value ζ is great, it means that the balance of the right and left principal muscles which are used in performing the leg raising is bad.

FIGS. 24(a) and 24(b) are explanatory views showing the detail evaluation based on the parameters [symbol] , [symbol] , and [symbol] of FIG. 22. Referring to FIG. 24(a), the meaning of the sum Ξ (= [symbol] + [symbol] ) is the same as that of the pelvis torsion ζ.

If the displacement width [symbol] of the lumbar part in the front direction in the fourth motion (the forward bending) exceeds 60 degrees, and furthermore the sum Ξ of the displacement width [symbol] of the lumbar part in the front direction and the displacement width [symbol] of the lumbar part in the back direction in the sixth motion (the thigh raising) is within the range 0-5 degrees, it is determined that the condition is a condition 1. If the displacement width [symbol] exceeds 60 degrees, and furthermore the sum Ξ is within the range 6-14 degrees, it is determined that the condition is a condition 2. If the displacement width [symbol] exceeds 60 degrees, and furthermore the sum Ξ is 15 degrees or more, it is determined that the condition is a condition 3.

Also, if the displacement width [symbol] is 60 degrees or below, and furthermore the sum Ξ is within the range 0-5 degrees, it is determined that the condition is a condition 4. If the displacement width [symbol] is 60 degrees or below, and furthermore the sum Ξ is within the range 6-14 degrees, it is determined that the condition is a condition 5. If the displacement width [symbol] is 60 degrees or below, and furthermore the sum Ξ is 15 degrees or more, it is determined that the condition is a condition 6.

As described above, each of the conditions 1 to 6 represents a condition of the lower body of the subject. For example, it is evaluated that the condition of the lower body is “bad” if the condition 1 is determined, it is evaluated that the condition of the lower body is “good” if the condition 2 is determined, it is evaluated that the condition of the lower body is “bad” if the condition 3 is determined, it is evaluated that the condition of the lower body is “singularly bad” if the condition 4 is determined, it is evaluated that the condition of the lower body is “bad” if the condition 5 is determined, and it is evaluated that the condition of the lower body is “singularly bad” if the condition 6 is determined. Also, although the displacement width [symbol] is compared with 60 degrees, it is not limited thereto. For example, it may be 50 degrees.

Referring to FIG. 24(b), the condition 1 represents that the muscle of the lumbar part is tight. The condition 2 represents that the pelvis angle is normal. The condition 3 represents that the muscles of buttocks are tight. The condition 4 represents that the lumbar part, the buttocks, the backs of the thighs, and the calves are tight. The condition 5 represents that the backs of the thighs and the calves are tight. The condition 6 represents that the buttocks, the backs of the thighs, and the calves are tight.

FIG. 25 is a flow chart showing an example of the detail evaluation process which is performed in step S330 of FIG. 16. Referring to FIG. 25, in step S501, the PC 7 calculates the shoulder skewness δ on the basis of the movable range A of the right arm and the movable range B of the left arm in performing the first motion. In step S503, the PC 7 determines whether or not the shoulder skewness δ is within ±5 degrees, the process proceeds to step S505 if the positive determination, conversely the process proceeds to step S507 if the negative determination. In step S505, the PC 7 determines that the skewness of the shoulders is within the normal range. On the other hand, in step S507, the PC 7 determines that the skewness of the shoulders is within the abnormal range.

In the case where it is determined that the skewness of the shoulders is normal, for example, the text, which expresses the sentences “The difference between the right and left shoulders is within the tolerable range. Try to have continuously daily lives in which the upper body is balanced.”, is contained in the print picture (step S341). On the other hand, in the case where it is determined that the skewness of the shoulders is abnormal, for example, the text, which expresses the sentences “The balance of the right and left shoulders is lost, and the right (left) shoulder is higher. The muscle from the base of the neck to the shoulder is apt to be tight. There is also a possibility that this causes the shoulder stiffness and the migraine.”, is contained in the print picture (step S341).

In step S509, the PC 7 calculates the balance ε of sides of a body on the basis of the movable range K of the upper body, the displacement width O of the lumbar part in the right direction, the movable range P of the upper body, and the displacement width S of the lumber part of the left direction in performing the third motion. In step S511, the PC 7 determines whether or not the balance ε of sides of a body is within ±4 degrees, the process proceeds to step S513 if the positive determination, conversely the process proceeds to step S515 if the negative determination. In step S513, the PC 7 determines that the balance of the sides of the body is within the normal range. On the other hand, in step S515, the PC 7 determines that the balance of the sides of the body is within the abnormal range.

In the case where it is determined that the balance of the sides of the body is normal, for example, the text, which expresses the sentences “The right/left balance of the waist is within the tolerable range. Try to avoid the motion such as the bearing of putting the center of the gravity over one foot when standing, as much as possible, which causes the disruption of the balance.”, is contained in the print picture (step S341). On the other hand, in the case where it is determined that the balance of the sides of the body is abnormal, for example, the text, which expresses the sentences “The right/left balance of the waist is lost. Compare the right and left waistlines in front of the mirror. Is there the difference?”, is contained in the print picture (step S341).

In step S517, the PC 7 calculates the pelvis right/left balance μ on the basis of the displacement width Θ of the lumbar part in the left direction, and the displacement width Σ of the lumber part of the right direction in performing the sixth motion. In step S519, the PC 7 determines whether or not the pelvis right/left balance μ is within ±6 degrees, the process proceeds to step S521 if the positive determination, conversely the process proceeds to step S523 if the negative determination. In step S521, the PC 7 determines that the right/left balance of the pelvis is within the normal range. In step S523, the PC 7 determines that the right/left balance of the pelvis is within the abnormal range.

In the case where it is determined that the right/left balance of the pelvis is normal, for example, the text, which expresses the sentences “The right/left balance of the pelvis is within the tolerable range. Try to avoid the motion such as the bearing of standing on a daily basis while putting the center of the gravity over one foot, and the cross-legged bearing, as much as possible, which causes the disruption of the balance.”, is contained in the print picture (step S341). On the other hand, in the case where it is determined that the right/left balance of the pelvis is abnormal, for example, the text, which expresses the sentences “The pelvis is apt to be displaced rightward (leftward). Don't you stand on a daily basis while putting the center of the gravity over one side, or sit cross-legged a daily basis? There is a possibility that the pelvis is displaced rightward (leftward).”, is contained in the print picture (step S341).

In step S525, the PC 7 acquires the displacement width D of the upper body in the back direction in performing the first motion as the pectoral rigidity v. In step S527, the PC 7 determines whether or not the pectoral rigidity v is 15 degrees or below, the process proceeds to step S529 if the positive determination, conversely the process proceeds to step S531 if the negative determination. In step S529, the PC 7 determines that the rigidity of the pectoral is within the normal range. On the other hand, in step S531, the PC 7 determines that the rigidity of the pectoral is within the abnormal range.

In the case where it is determined that the rigidity of the pectoral is normal, for example, the text, which expresses the sentences “The flexibility of the muscle of the breast is within the tolerable range. But, as the muscle of the breast is apt to tight, try to keep the flexibility by taking in the stretching and so on.”, is contained in the print picture (step S341). On the other hand, in the case where it is determined that the rigidity of the pectoral is abnormal, for example, the text, which expresses the sentences “The muscle of the breast is rigid. If it continues getting rigid, you slouch, and therefore there is a possibility that this causes the so-called stoop. Also, there is a possibility that this causes the shoulder stiffness.”, is contained in the print picture (step S341).

In step S533, the PC 7 calculates the back muscle flexibility ρ on the basis of the displacement width Ω of the upper body in the front direction and the displacement width [symbol] of the lumber part in the front direction in performing the fourth motion. In step S535, the PC 7 determines whether or not the back muscle flexibility ρ is 70 degrees or more, the process proceeds to step S537 if the positive determination, conversely the process proceeds to step S539 if the negative determination. In step S537, the PC 7 determines that the flexibility of the muscle of the back is within the normal range. On the other hand, in step S539, the PC 7 determines that the flexibility of the muscle of the back is within the abnormal range.

In the case where it is determined that the flexibility of the muscle of the back is normal, for example, the text, which expresses the sentences “The flexibility of the back (backbone) is within the tolerable range. As the move is apt to be less with age, try to keep the flexibility by exercising steadily.”, is contained in the print picture (step S341). On the other hand, in the case where it is determined that the flexibility of the muscle of the back is abnormal, for example, the text, which expresses the sentences “The flexibility of the back (backbone) lacks. Although it may seem to have a good posture seemingly, the flexibility of the lumbar part lacks, and therefore you have the condition in which the tiredness is apt to be accumulate in the lumbar pat.”, is contained in the print picture (step S341).

In step S541, the PC 7 calculates the shoulder defect 6 on the basis of the displacement width E of the upper body in the left direction and the displacement width F of the upper body in the right direction in performing the first motion. In step S543, the PC 7 determines whether or not the shoulder defect σ is within ±2 degrees, the process proceeds to step S545 if the positive determination, conversely the process proceeds to step S547 if the negative determination. In step S545, the PC 7 determines that the shoulder is within the normal range. On the other hand, in step S547, the PC 7 determines that the shoulder is within the abnormal range.

In the case where it is determined that the shoulder is normal, for example, the text, which expresses the sentences “Both of the bladebones and the arms normally function.”, is contained in the print picture (step S341). On the other hand, in the case where it is determined that the shoulder is abnormal, for example, the text, which expresses the sentences “The body is inclined due to protect the arm and shoulder. Is there something wrong with the shoulder? If it is left, there is a possibility that this causes even the headache and backache.”, is contained in the print picture (step S341).

In step S549, the PC 7 calculates the lateral muscle flexibility τ on the basis of the movable range K of the upper body, the displacement width O of the lumbar part in the right direction, the movable range P of the upper body, and the displacement width S of the lumber part in the left direction in performing the third motion. In step S551, the PC 7 determines whether or not the lateral muscle flexibility τ is 30 degrees or more, the process proceeds to step S553 if the positive determination, conversely the process proceeds to step S555 if the negative determination. In step S553, the PC 7 determines that the flexibility of the lateral muscles is within the normal range. On the other hand, in step S555, the PC 7 determines that the flexibility of the lateral muscles is within the abnormal range.

In the case where it is determined that the flexibility of the lateral muscles is normal, for example, the text, which expresses the sentences “You have no problem with the flexibility of sides of the body. Continue exercising so as to keep the current condition.”, is contained in the print picture (step S341). On the other hand, in the case where it is determined that the flexibility of the lateral muscles is abnormal, for example, the text, which expresses the sentences “The flexibility of the side of the body lacks. The muscle from the flank to the back is apt to be tight, and therefore there is a possibility that this causes even the backache.”, is contained in the print picture (step S341).

In step S557, the PC 7 calculates the upper body right/left torsion ξ on the basis of the displacement widths α and γ of the upper body in the front direction in performing the third motion. In step S559, the PC 7 determines whether or not the upper body right/left torsion ξ is within ±3 degrees, the process proceeds to step S561 if the positive determination, conversely the process proceeds to step S563 if the negative determination. In step S561, the PC 7 determines that the right/left torsion of the upper body is within the normal range. On the other hand, in step S563, the PC 7 determines that the right/left torsion of the upper body is within the abnormal range.

In the case where it is determined that the right/left torsion of the upper body is normal, for example, the text, which expresses the sentences “The right/left torsion of the upper body is within the tolerable range. But, the right and left balance is readily lost depending on the arm holding the bag, the standing manner, and the sitting manner. Try to have the balanced daily lives.”, is contained in the print picture (step S341). In the case where it is determined that the right/left torsion of the upper body is abnormal, for example, the text, which expresses the sentences “There is the right/left torsion of the upper body. Doesn't the right (left) shoulder twist forward? The torsion may cause the symptoms such as the backache and the shoulder stiffness.”, is contained in the print picture (step S341).

In step S565, the PC 7 calculates the pelvis torsion ζ on the basis of the displacement width [symbol] of the lumbar part in the front direction and the displacement width [symbol] of the lumber part of the back direction in performing the sixth motion. In step S567, the PC 7 determines whether or not the pelvis torsion ζ is within ±5 degrees, the process proceeds to step S569 if the positive determination, conversely the process proceeds to step S571 if the negative determination. In step S569, the PC 7 determines that the torsion of the pelvis is within the normal range. On the other hand, in step S571, the PC 7 determines that the torsion of the pelvis is within the abnormal range.

In the case where it is determined that the torsion of the pelvis is normal, for example, the text, which expresses the sentences “The torsion of the pelvis is within the tolerable range. The pelvis readily twists if you continue sitting cross-legged, sitting with your legs folded sideways, and so on. Try to have the balanced daily lives.”, is contained in the print picture (step S341). On the other hand, in the case where it is determined that the torsion of the pelvis is abnormal, for example, the text, which expresses the sentences “There is the torsion of the pelvis. The left (right) pelvis is inclined in the more back direction. The right (left) stride may be larger than the left (right) stride.”, is contained in the print picture (step S341).

Incidentally, in the above examples of the text contents, there is the part where either “right” or “left” is chosen to use (the alternative being provided in parentheses). In this case, either “right” or “left” is chosen in accordance with the posture pattern.

In step S573, the PC 7 calculates the parameter Ξ on the basis of the displacement width [symbol] of the lumbar part in the front direction and the displacement width [symbol] of the lumber part of the back direction in performing the sixth motion. In step S575, the PC 7 determines the condition (any one of the conditions 1 to 6) on the basis of the parameter Ξ and the displacement width [symbol] of the lumbar part in the front direction in performing the fourth motion.

In the case where the condition 1 is determined, for example, the text, which expresses the sentences “The lumbar part and the muscles of the front sides of the thighs are tight. The lumbar part too warps, and there is a possibility that this causes the backache.”, is contained in the print picture (step S341). In the case where the condition 2 is determined, for example, the text, which expresses the sentences “You have no problem with the lumbar part, and the muscles of the buttocks, the thighs, the calves, and the back. Continue the stretching and so on so as to keep this condition.”, is contained in the print picture (step S341).

In the case where the condition 3 is determined, for example, the text, which expresses the sentences “The flexibility of the glutei lacks. The movement of the hip joints is restricted, the blood flow of the legs and the flow of the lymph are impaired, and there is a possibility that this causes the cold and swelling of feet.”, is contained in the print picture (step S341). In the case where the condition 4 is determined, for example, the text, which expresses the sentences “The entire back side of the lower body is tight. The stride is little, the exercise efficiency is impaired, and therefore there is a possibility that the fat of the body is hard to be burnt.”, is contained in the print picture (step S341).

In the case where the condition 5 is determined, for example, the text, which expresses the sentences “The muscle from the back of the thigh to the calf is tight. Don't you have a sedentary lifestyle, or often wear high-heeled shoes if you are a female? It is said that the calf is the second heart. Especially, try to have daily lives in which the flexibility of the calf (ankle) is maintained.”, is contained in the print picture (step S341). In the case where the condition 6 is determined, for example, the text, which expresses the sentences “The muscle from the buttock to the back side of the thigh and the calf is tight. The activity of the muscle of the lower body is reduced, the tiredness material and waste material are apt to be accumulated, and there is a possibility that this causes swelling and cellulite. Don't you have a sedentary lifestyle, or often wear high-heeled shoes?”, is contained in the print picture (step S341).

By the way, as described above, in accordance with the present embodiment, the movable ranges AL and AR of the arms of the subject 1, and the displacement widths WL, WR, and CF are detected, and then the posture of the subject 1 can be classified on the basis of them. Specifically, the tilt between the right and left shoulders can be obtained by measuring the movable range AL of the left arm on the basis of the output of the sensor unit 3L and measuring the movable range AR of the right arm on the basis of the output of the sensor unit 3R. Also, the position of the load and the tilt of the pelvis can be deduced by measuring the displacement widths WL, WR, and CF of the pelvis in the front, back, right, and left directions on the basis of the output of the sensor unit 3W. Then, as described above, the posture of the subject 1 can be classified into any one of the 8 patterns (see Table 1).

Also, in the present embodiment, the state angle displaying sections 105L, 105R, and 105U2, and the displacement angle displaying sections 105U1 and 105B are displayed on the monitor 43. Thus, the subject 1 can monitor the movement of the his/her own measured parts on a real-time basis, and the measurer such as a doctor can monitor the movement of the measured parts of the subject 1 on a real-time basis.

Further, in the present embodiment, the instruction section 103 is displayed on the monitor 43. Thus, since the motion which is required to measure is shown with the image, it is possible to instruct all the subjects the uniform motion. Also, the subject can easily recognize the motion which he/she has to perform.

Still further, in the present embodiment, in addition to measuring the posture, the exercise for correcting the posture is shown by the exercise prescription screen, whereby it is possible to seamlessly link the measurement of the posture and the correction exercise, and therefore it is convenient for the subject 1.

Also, in the present embodiment, the vicarious movement accompanying the predetermined motion is measured, and then the posture of the subject is evaluated by using it as an index (the pelvis right/left balance μ, the pectoral rigidity v, the shoulder defect σ, and the pelvis torsion ζ in the above example).

Even if the physical conditions of the parts of the body such as the muscles and joints are normal, the vicarious movement usually accompanies motion. However, if the vicarious movement is too great, it means that a part (hereinafter referred to as a “principal part”) which originally has to perform motion does not function normally, or there is a part (hereinafter referred to as a “encumbering part”) which encumbers the function of the principal part.

Accordingly, it is possible to determine whether or not the principal part normally functions, or grasp and evaluate the extent of the normal or abnormal on the basis of the extent of the vicarious movement accompanying the predetermined motion. Also, it is possible to determine whether or not the encumbrance by the encumbering part occurs (is within the normal range), or evaluate the extent of the encumbrance, i.e., the encumbering part on the basis of the extent of the vicarious movement. As the encumbrance by the encumbering part becomes greater, the vicarious movement becomes greater so as to absorb and eliminate the encumbrance. On the other hand, as the encumbrance by the encumbering part becomes smaller, the vicarious movement becomes smaller.

Further, since the difference between the vicarious movements accompanying the bilaterally symmetric motion is obtained, it is possible to determine the defect of any one of the right and left or evaluate the balance of the right and left with regard to the part performing the vicarious movement (the pelvis right/left balance μ and the shoulder defect σ in the above example).

Also, in the present embodiment, since the vicarious movement (so to speak, the noise) is subtracted from the predetermined motion as measured, only the motion by the principal part can be extracted, and therefore it is possible to accurately evaluate the principal part (the balance ε of sides of a body and the pelvis torsion ζ in the above example).

Further, since the vicarious movement is subtracted by the calculation after performing the predetermined motion including the vicarious movement, it is possible to reduce a cost and simply measure by dispensing with equipment for restraining parts of the body as the prior art.

Still further, since the difference between the bilaterally symmetric movements which are performed by the principal parts is obtained, it is possible to determine the defect of any one of the right and left, or evaluate the balance of the right and left with regard to the principal parts (the balance ε of sides of a body in the above example).

Also, in the present embodiment, the sensor units 3R, 3L, 3C, and 3W are mounted on the subject so as not to positively restrict the predetermined motion and the vicarious movement. In this way, since the predetermined motion and the vicarious movement are not restricted, it is possible to measure the vicarious movement under a natural situation, i.e., a situation where the subject is usually put in a regular life. As the result, it is possible to evaluate the condition of the posture of the subject under the natural situation. In contrast, in Patent Document 2, the predetermined parts of the subject are restrained, and therefore this situation is a special situation for measuring.

Further, the sensor units 3C and 3W for measuring the predetermined motion and the vicarious movement are arranged on the central line 2 which divides the subject into the right and left. Thus, it is possible to effectively measure the predetermined motion and the vicarious movement. Because, it is possible to measure not only the motion of the part on the central line 2 (the predetermined motion and the vicarious movement) but also the bilaterally symmetric motion with regard to the central line (the predetermined motion and the vicarious movement).

Incidentally, an example of mounting the sensor units 3R, 3L, 3C, and 3W will be described. The sensor units 3R, 3L, 3C, and 3W are attached to elastic belts. Then, the subject wears these belts. In this case, since the belts are elastic, the belts constrict the body of the subject, and therefore it might be said that the belts restrict the move of the subject. However, the constriction is a function for mounting the sensor units 3R, 3L, 3C, and 3W, and is not an absolute requirement for the detection by the sensor units 3R, 3L, 3C, and 3W. Therefore, it can not be said that the move is positively restricted in this case. On the other hand, as shown in Patent Document 2, in the case where the restraint of the parts of the body is an absolute requirement for the measurement, it can be said that the move is positively restricted.

[First Modification]

A body condition evaluation system according to a first modification of the above embodiment will be described. The first modification mainly differs in a classifying method of a posture pattern, parameters using for the calculation of the posture barometers of FIG. 23, and a scale of evaluation from the above embodiment. Both have the same hardware configuration. In what follows, the different points will be mainly described.

First, the classification of the posture pattern will be described.

The posture pattern is determined on the basis of a barometer SB representing the balance of the right shoulder and the left shoulder, a barometer WB representing the balance of the right side and the left side of the lumbar part, a barometer BB representing a condition of the back (backbone), and a barometer TB representing the balance of the great trochanters.

The calculating formula of each barometer is as follows. See FIG. 22 with regard to each parameter in the formula.

SB=A−B

WB=Σ−Σ

BB=

[symbol]

TB=O−S

The PC 7 determines that the balance of the right and left shoulders is normal if −4≦SB≦4. The PC 7 determines that the right shoulder is higher if SB>4. The movable range A of the right arm is considerably larger than the movable range B of the left arm (the absolute value). This indicates that the right shoulder is high relative to the left shoulder. On the other hand, the PC 7 determines that the left shoulder is higher if SB<−4. The movable range B of the left arm is considerably larger than the movable range A of the right arm (the absolute value). This indicates that the left shoulder is high relative to the right shoulder.

The PC 7 determines that the right/left balance of the pelvis is normal if −4≦W≦4. The PC 7 determines that the pelvis inclines upward to the left if WB>4. The right displacement width Σ is considerably larger than the left displacement width Θ (the absolute value). This indicates that the left side of the pelvis is high relative to the right side thereof (a left waist ascent) and the pelvis is displaced (is shifted) leftward (a left load). On the other hand, the PC 7 determines that the pelvis inclines upward to the right if WB<−4. The left displacement width Θ is considerably larger than the right displacement width Σ (the absolute value). This indicates that the right side of the pelvis is high relative to the left side thereof (a right waist ascent) and the pelvis is displaced (is shifted) rightward (a right load).

The PC 7 determines that the condition of the back is normal if 50≦BB≦60. The PC 7 determines that the back warps backward if BB>60. The displacement width [symbol] exceeds 60 degrees. This indicates that the pelvis inclines forward (a forward tilt), and whereby the lower back warps (the back warps backward). That is, the chest is thrown out and the lower back warps, and the knees extend completely. In this posture, the body weight is apt to be applied to the tiptoes. On the other hand, the PC 7 determines that the back is the stoop if BB<50. The displacement width [symbol] is smaller than 50 degrees. This indicates that the pelvis inclines backward (a backward tilt), and the back is the stoop. That is, the back is rounded, and the knees are slightly apt to bend. In this posture, the body weight is apt to be applied to the heels.

The PC 7 determines that the great trochanters incline upward to the right if the pelvis does not incline upward to the right and furthermore TB>3. The PC 7 determines that the great trochanters incline upward to the left if the pelvis does not incline upward to the left and furthermore TB<−3. In cases other than these, the PC 7 determines that the balance of the great trochanters is normal.

The PC 7 classifies the posture of the subject (the determination of the posture pattern) on the basis of such determination results (the balance of the right and left shoulders, the right/left balance of the pelvis, the condition of the back, and the balance of the great trochanters), and generates the human body image corresponding to the posture to include it in a total result screen and a print picture as described below. That is, the PC 7 determines one pattern from among 63 posture patterns on the basis of the determination results. Incidentally, there are the requirements other than the barometer TB with regard to the balance of the great trochanters, and therefore all the posture pattern is not 81 patterns but 63 patterns.

Also, the PC 7 specifies the regions of the body on which fat is apt to be put and the regions of the body whose muscles are apt to go tight on the basis of the determination results, and gives colors corresponding to them to the human body image. Further, the PC 7 determines whether or not the strides of the subject are balanced between right and left, and deduces which of the right stride and the left stride is greater if they are not balanced.

In what follows, these details will be described using Tables 2 to 6. In Tables 2 to 5, the letter “F” represents that the fat is apt to be put on while the letter “M” represents that the muscle is apt to go tight. Also, in Table 6, the symbol “*” represents any condition.

TABLE 2

| | Part | | | |
|---|---|---|---|---|
| | Right | Left | | |
| Shoulder | Shoulder | Shoulder | Right Flank | Left Flank |
| Upward To Left | F | M | M | F |
| Upward To Right | M | F | F | M |

As shown in Table 2, if the PC 7 determines that the shoulders incline upward to the left, the PC 7 determines (deduces) that the muscles of the left shoulder and the right flank are apt to go tight, and the fat is apt to be put on the right shoulder and the left flank. On the other hand, if the PC 7 determines that the shoulders incline upward to the right, the PC 7 determines (deduces) that the muscles of the right shoulder and the left flank are apt to go tight, and the fat is apt to be put on the left shoulder and the right flank.

In this way, it is possible to simply deduce the parts on which fat is apt to be put and the parts whose muscles are apt to go tight in the shoulder and the flank only by measuring the condition of the shoulders.

TABLE 3

| | Part | | | |
|---|---|---|---|---|
| | Right | Left | | |
| Pelvis | Waist | Waist | Right Flank | Left Flank |
| Upward To Right | F | M | M | F |
| Upward To Left | M | F | F | M |

As shown in Table 3, if the PC 7 determines that the pelvis inclines upward to the right, the PC 7 determines (deduces) that the muscles of the left side of the waist and the right flank are apt to go tight, and the fat is apt to be put on the right side of the waist and the left flank. On the other hand, if the PC 7 determines that the pelvis inclines upward to the left, the PC 7 determines (deduces) that the muscles of the right side of the waist and the left flank are apt to go tight, and the fat is apt to be put on the left side of the waist and the right flank.

In this way, it is possible to simply deduce the parts on which fat is apt to be put and the parts whose muscles are apt to go tight in the waist and the flank only by measuring the condition of the pelvis.

TABLE 4

| | Part | | | |
|---|---|---|---|---|
| | Right | Left | Inner Side Of | Inner Side Of |
| Great Trochanter | Waist | Waist | Right Thigh | Left Thigh |
| Upward To Left | F | M | M | F |
| Upward To Right | M | F | F | M |

As shown in Table 4, if the PC 7 determines that the great trochanters incline upward to the left, the PC 7 determines (deduces) that the muscles of the left side of the waist and the inner side of the right thigh are apt to go tight, and the fat is apt to be put on the right side of the waist and the inner side of the left thigh. On the other hand, if the PC 7 determines that the great trochanters incline upward to the right, the PC 7 determines (deduces) that the muscles of the right side of the waist and the inner side of the left thigh are apt to go tight, and the fat is apt to be put on the left side of the waist and the inner side of the right thigh.

In this way, it is possible to simply deduce the parts on which fat is apt to be put and the parts whose muscles are apt to go tight in the waist and the inner side of the thigh only by measuring the condition of the great trochanters.

TABLE 5

| | Part | | | | | |
|---|---|---|---|---|---|---|
| | | | | | Front | Back |
| Backbone | Breast | Back | Belly | Buttock | Thigh | Thigh |
| Warped Back | — | M | F | F | M | F |
| Stoop | M | F | — | M | F | M |

As shown in Table 5, if the PC 7 determines that the back (backbone) warps backward, the PC 7 determines (deduces) that the muscle of the back and the muscles of the front sides of the thighs are apt to go tight, and the fat is apt to be put on the belly, the buttocks, and the back sides of the thighs. On the other hand, if the PC 7 determines that the back (backbone) is the stoop, the PC 7 determines (deduces) that the muscles of the breast, the buttocks, and the back sides of the thighs are apt to go tight, and the fat is apt to be put on the back, and the front sides of the thighs.

In this way, it is possible to simply deduce the parts on which fat is apt to be put and the parts whose muscles are apt to go tight in the breast, the back, the belly, the buttocks, and the front sides and the back sides of the thighs only by measuring the condition of the pelvis and determining the condition of the back (backbone).

As described above, the PC 7 determines (deduces) that the fat is apt to be put on the part extended by the skewness of the body. On the other hand, the PC 7 determines (deduces) that the muscle of the part shriveled by the skewness of the body is apt to go tight

TABLE 6

| | Great | Part |
|---|---|---|
| Pelvis | Trochanter | Stride |
| Upward To Right | * | Large Right |
| Upward To Left | * | Large Left |
| Normal | Upward To Left | Large Right |
| Normal | Upward To Right | Large Left |

As shown in Table 6, if the PC 7 determines that the pelvis inclines upward to the right, the PC 7 determines (deduces) that the right stride becomes large. If the PC 7 determines that the pelvis inclines upward to the left, the PC 7 determines (deduces) that the left stride becomes large. However, even if the PC 7 determines that the pelvis is normal, the PC 7 determines (deduces) that the right stride becomes large if the PC 7 determines that the great trochanters incline upward to the left, and the PC 7 determines (deduces) that the left stride becomes large if the PC 7 determines that the great trochanters incline upward to the right. Also, in cases other than these, the PC 7 determines that the balance of the strides is normal.

In this way, it is possible to simply deduce the stride only by measuring the condition of the pelvis. Further, since the condition of the great trochanters is reflected, it is possible to deduce the stride more accurately.

Incidentally, the barometer SB is the same as (AR−AL) of the above embodiment, the barometer WB is the same as (WR−WL) of the above embodiment, and the barometer BB is the same as CF of the above embodiment. Consequently, in the above embodiment, the part whose muscle is apt to go tight and the part on which the fat is apt to be put are determined on the basis of Table 2, Table 3, and Table 5. Besides, the section of “Shoulder” in Table 1 corresponds to the section of “Shoulder” in Table 2, the section of “Load” in Table 1 corresponds to the section of “Pelvis” in Table 3, and the section of “Pelvis” in Table 1 corresponds to the section of “Backbone” in Table 5. However, the “Right Load” and the “Left Load” in Table 1 correspond to the “Upward To Right” and the “Upward To Left” in Table 3 respectively. Also, the “Forward Tilt” and the “Backward Tilt” in Table 1 correspond to the “Warped Back” and the “Stoop” in Table 5 respectively.

Further, in the above embodiment, the balance of the strides is determined referring to Table 6 on the basis of (WR−WL), i.e., the barometer WB. In this case, the great trochanters are not reflected.

Next, the posture barometers will be described.

The posture barometers includes shoulder skewness δ, balance ε of sides of a body (lateral muscle balance), pelvis right/left balance μ, pectoral rigidity v, back muscle flexibility ρ, a shoulder defect σ, lateral muscle flexibility τ, upper body right/left torsion ξ, and pelvis torsion ζ. These are expressed by the following formulae using the parameters of FIG. 22.

δ=A−B

ε=(RR−O)−(LL−S)

μ=Θ−Σ

v=D

ρ=U−

[symbol]

σ=E−F

σ=MIN((RR−O),(LL−S))

ξ=α−γ

ζ=( [symbol] + [symbol] )_{R}−( [symbol] + [symbol] )_{L}

Incidentally, in the formula of the lateral muscle flexibility τ, the term “MIN” indicates that the smaller one of (RR−O) and (LL−S) is used as the lateral muscle flexibility τ.

The points different from the above embodiment are the balance ε of sides of a body, the back muscle flexibility ρ, and the lateral muscle flexibility τ.

That is, with regard to the balance ε of sides of a body and the lateral muscle flexibility τ, although the above embodiment uses the parameters K and P, the first modification uses the parameters RR and LL. Because, the subject may be accompanied by the unignorable forward tilt in performing the lateral bending, and therefore the displacement width LL in the left direction and the displacement width RR in the right direction are used in place of the movable range P in the left direction and the movable range K in the right direction so as to eliminate the influence of the forward tilt.

The balance ε of sides of a body is a value obtained by subtracting a value (RR−O) from a value (LL−S), and represents the balance of the right/left flexibility of the lateral muscles of the upper body. The value (RR−O) is obtained by subtracting the pelvis angle (the displacement width of the lumbar part in the right direction) O from the displacement width RR of the upper body in the right direction when the upper body is bent rightward. The value (LL−S) is obtained by subtracting the pelvis angle (the displacement width of the lumbar part in the left direction) S from the displacement width LL of the upper body in the left direction when the upper body is bent leftward. When it is tried to bend the upper body, if the lateral muscle does not have the flexibility, the upper body is bent by moving the pelvis (vicarious movement), and therefore the pelvis angle, i.e., the vicarious movement is subtracted from the displacement width of the upper body so as to obtain the real bend of the upper body by the lateral muscle.

The lateral muscle flexibility τ is the smaller one of the values (RR−O) and (LL−S). The value (RR−O) is obtained by subtracting the pelvis angle (the displacement width of the lumbar part in the right direction) O from the displacement width RR of the upper body in the right direction when performing the lateral bending rightward. The value (LL−S) is obtained by subtracting the pelvis angle (the displacement width of the lumbar part in the left direction) S from the displacement width LL of the upper body in the left direction when performing the lateral bending leftward. The reason for subtracting the pelvis angle from the displacement width of the upper body is the same as that of the balance ε of sides of a body

Also, with regard to the back muscle flexibility ρ, the parameter Ω is used in the above embodiment while the parameter U is used in the first modification. With regard to this point, it does not make much difference in the evaluation substantially.

The back muscle flexibility ρ is a value (U− [symbol] ) obtained by subtracting the forward-tilt angle [symbol] of the pelvis (the displacement width [symbol] of the lumbar part in the front direction) from the movable range U of the upper body when performing the forward bending. In this way, the real forward bending by the muscle of the back is obtained by eliminating the bending by the pelvis. The value ρ hereby represents the flexibility of the back muscle.

By the way, although the evaluation of each barometer is classified into either normal or abnormal in the above embodiment, the evaluation of each barometer is classified on four-point scale (Good, Normal, Bad, and Worst) in the first modification.

The absolute values of the above barometers are collectively represented by the reference BA. In such case, with regard to the barometers δ, ε, μ, v, σ, ξ, and ζ, the evaluation of “Good” is given if BA≦C0, the evaluation of “Normal” is given if C0<BA≦C1, the evaluation of “Bad” is given if C1<BA≦C2, and the evaluation of “Worst” is given if C2<BA. In this case, constants C0 to C2 are a positive integer, and are given for each barometer through an experiment and a trial and error process. Also, the relation C0<C1<C2 is satisfied.

Besides, with regard to the barometers ρ and τ, the evaluation of “Good” is given if BA≧C4, the evaluation of “Normal” is given if C5≦BA<C4, the evaluation of “Bad” is given if C6≦BA<C5, and the evaluation of “Worst” is given if BA<C6. In this case, constants C4 to C6 are a positive integer, and are given for each barometer through an experiment and a trial and error process. Also, the relation C4>C5>C6 is satisfied.

Incidentally, the evaluation of the condition of the lower body is the same as that of the above embodiment (see FIG. 24).

Next, a total result screen and a printed result according to the first modification will be described referring to the figures.

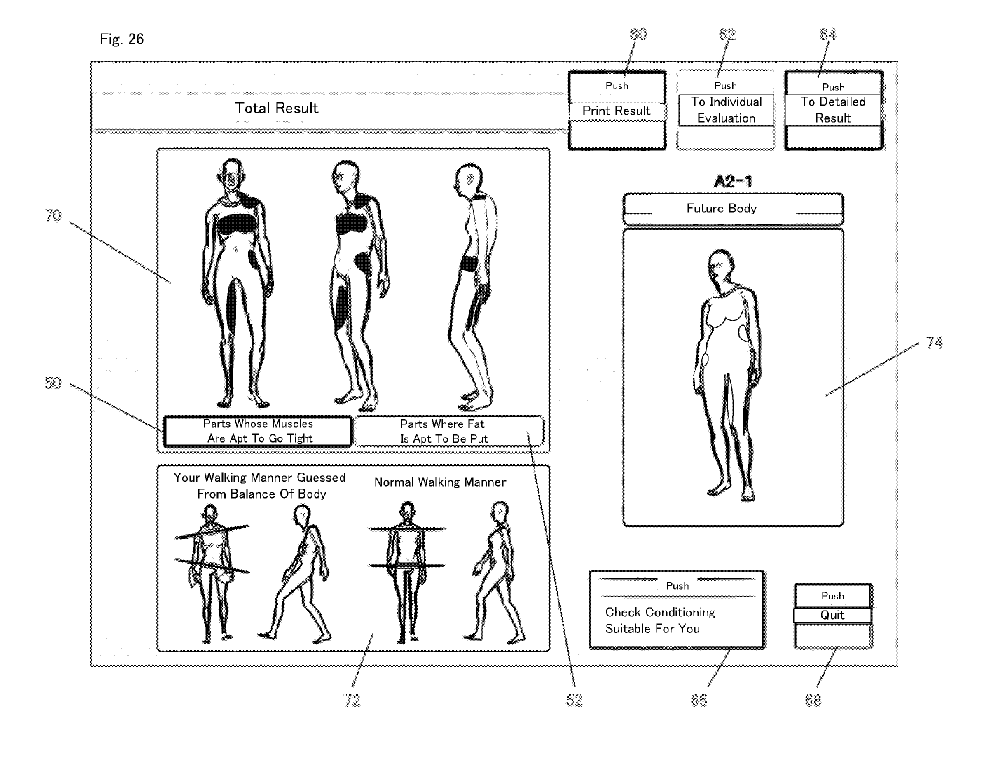

FIG. 26 is a view showing a first displayed example of the total result screen in accordance with the first modification example of the embodiment of the present invention. Referring to FIG. 26, this total result screen has the configuration similar to the total result screen of FIG. 17. The different points will be mainly described.

A first frame 70 contains buttons 50 and 52. When an operator moves a cursor to the button 50 and clicks, a human body image corresponding to the posture of the subject as determined in the above manner is displayed in the first frame 70. The parts whose muscles are apt to go tight are shown by a first color (the black color in the figure) in this human body image.

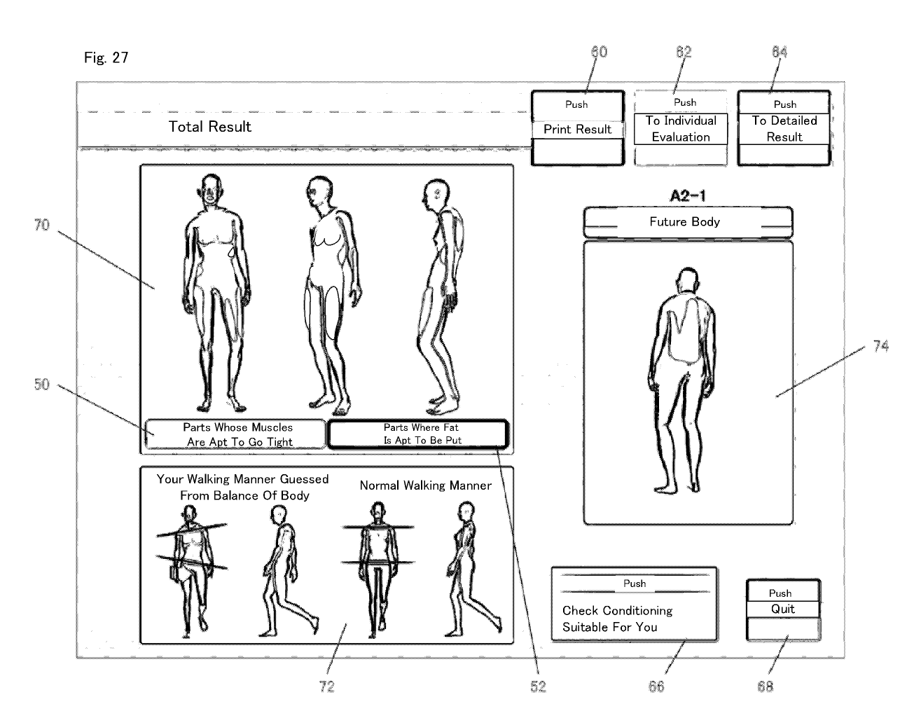

FIG. 27 is a view showing a second displayed example of the total result screen. Referring to FIG. 27, when the operator moves the cursor to the button 52 and clicks, a human body image corresponding to the posture of the subject as determined in the above manner is displayed in the first frame 70. The parts on which fat is apt to be put are shown by a second color (the white color region surrounded by the black line in the figure) in this human body image.

As described above, the first frame displays any one of the human body image representing the parts whose muscles are apt to go tight and the human body image representing the parts on which the fat is apt to be put in accordance with the operation of the buttons 50 and 52.

Also, a walking manner as guessed on the basis of the posture of the subject as determined in the above manner is shown in the second frame 72 by animation. In this case, the human body image is animated on the basis of the information of the stride which is determined on the basis of the posture of the subject. Further, when the strides of the right and left are not balanced, the arrows which have the colors and widths different from each other are displayed so as to distinguish between the leg whose stride is larger and the other leg.

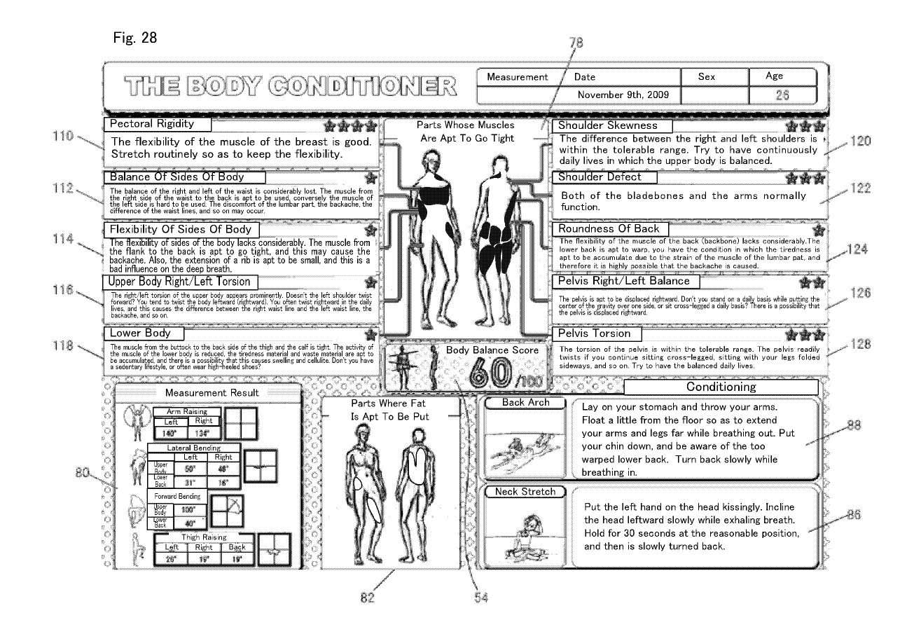

FIG. 28 is a view showing an example of the printed result in accordance with the first modification. Referring to FIG. 28, this printed result has the configuration similar to the printed result of FIG. 21. The different points will be mainly described.

This printed screen contains a score displaying section 54. The score displaying section 54 displays a score BP which indicates extent of the balance of the body of the subject. The score BP is calculated on the basis of the posture barometers δ, ε, μ, v, ρ, σ, τ, ξ and ζ of the subject, and the condition of the lower body (see FIG. 24). Specifically, since each barometer is indicated on four-point scale, a point is assigned to each scale. Also, since the condition of the lower body is indicated on three-point scale, a point is assigned to each scale. Then, the score BP of the subject is calculated on the basis of the sum of all the barometers and the condition of the lower body.

For example, with regard to each barometer, four points are assigned to the evaluation “Good”, three points are assigned to the evaluation “Normal”, two points are assigned to the evaluation “Bad”, and one point is assigned to the evaluation “Worst”. With regard to the condition of the lower body, four points are assigned to the evaluation “Good”, two points are assigned to the evaluation “Bad”, and one point is assigned to the evaluation “Worst”. In that case, BP=20+Σ(Pj*2). Incidentally, the suffix j=0 to 9, and the symbol Σ represents a summation from j=0 to j=9. The variable Pj indicates the barometer or the condition of the lower body.

Also, in the first modification, the frames 110 to 128 corresponding to the fourth frame 84 of FIG. 21 are contained. The frames 110, 112, 114, 116, 118, 120, 122, 124, 126, and 128 represent comments based on v, ε, τ, ξ, the condition of the lower body, δ, σ, ρ, μ, and ζ respectively.

Next, the flow of the process of the PC 7 according to the first modification will be described using the flowcharts.

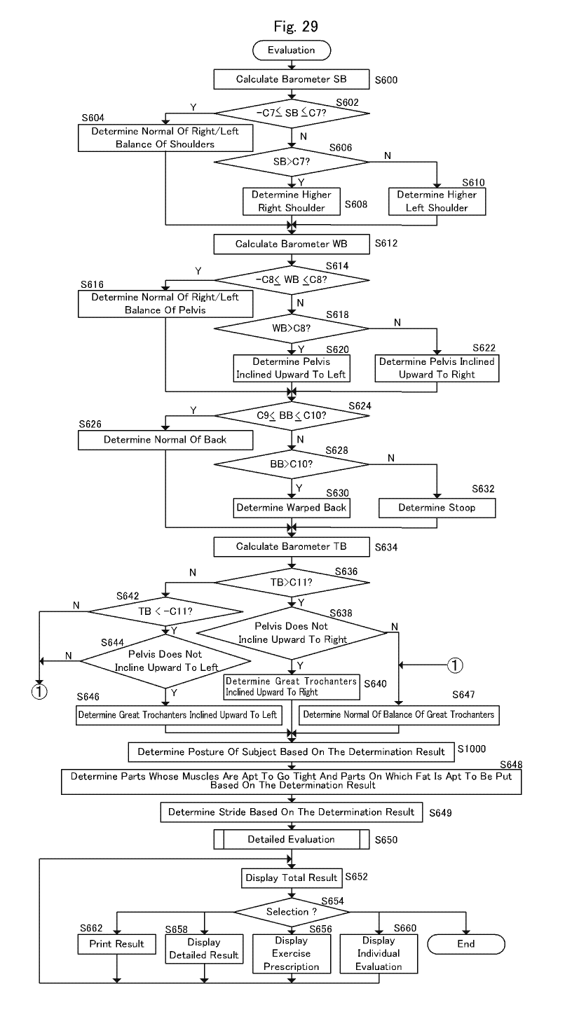

FIG. 29 is a flow chart showing an evaluation process by the PC 7 in accordance with the first modification. Referring to FIG. 29, in step S600, the PC 7 calculates the barometer SB on the basis of the parameters A and B. In step S602, the PC 7 determines whether or not the barometer SB is not less than the constant (−C7) and furthermore is not more than the constant C7, the process proceeds to step S604 if it is within such range, otherwise the process proceeds to step S606. In step S604, the PC 7 determines that the balance of the right and left shoulders is normal, and then proceeds to step S612.

On the other hand, in step S606, the PC 7 determines whether or not the barometer SB exceeds the constant C7, the process proceeds to step S608 if it exceeds, otherwise, i.e., if the barometer SB is below the constant (−C7), the process proceeds to step S610. In step S608, the PC 7 determines that the right shoulder is higher. On the other hand, in step S610, the PC 7 determines that the left shoulder is higher.

In step S612, the PC 7 calculates the barometer WB on the basis of the parameters Σ and Θ. In step S614, the PC 7 determines whether or not the barometer WB is not less than the constant (−C8) and furthermore is not more than the constant C8, the process proceeds to step S616 if it is within such range, otherwise the process proceeds to step S618. In step S616, the PC 7 determines that the right/left balance of the pelvis is normal, and then proceeds to step S624.

On the other hand, in step S618, the PC 7 determines whether or not the barometer WB exceeds the constant C8, the process proceeds to step S620 if it exceeds, otherwise, i.e., if the barometer WB is below the constant (−C8), the process proceeds to step S622. In step S620, the PC 7 determines that the pelvis inclines upward to the left. On the other hand, in step S622, the PC 7 determines that the pelvis inclines upward to the right.

In step S624, the PC 7 determines whether or not the barometer BB is not less than the constant C9 and furthermore is not more than the constant C10, the process proceeds to step S626 if it is within such range, otherwise the process proceeds to step S628. In step S626, the PC 7 determines that the condition of the back is normal, and then proceeds to step S634.

On the other hand, in step S628, the PC 7 determines whether or not the barometer BB exceeds the constant C10, the process proceeds to step S630 if it exceeds, otherwise, i.e., if the barometer BB is below the constant C9, the process proceeds to step S632. In step S630, the PC 7 determines that the back warps backward. On the other hand, in step S632, the PC 7 determines that the back is the stoop.

In step S634, the PC 7 calculates the barometer TB on the basis of the parameters O and S. In step S636, the PC 7 determines whether or not the barometer TB exceeds the constant C11, the process proceeds to step S638 if it exceeds, otherwise, the process proceeds to step S642. In step S638, the PC 7 determines whether or not it is determined that the pelvis does not incline upward to the right, the process proceeds to step S640 if it does not incline upward to the right, otherwise, the process proceeds to step S647. In step S640, the PC 7 determines that the great trochanters incline upward to the right. On the other hand, in step S647, the PC 7 determines that the balance of the great trochanters is normal.

In step S642, the PC 7 determines whether or not the barometer BB is below the constant (−C11), the process proceeds to step S644 if it is below, otherwise, the process proceeds to step S647. In step S644, the PC 7 determines whether or not it is determined that the pelvis does not incline upward to the left, the process proceeds to step S646 if it does not incline upward to the left, otherwise, the process proceeds to step S647. In step S646, the PC 7 determines that the great trochanters incline upward to the left.

In step S1000 after step S646, S640 or S647, the PC 7 determines the posture of the subject on the basis of the evaluations (steps S600 to S647) of the barometers SB, WB, BB, and TB of the subject (selects one from the 63 posture patterns). In step S648 after step S1000, the PC 7 determines the parts whose muscles are apt to go tight and the parts on which the fat is apt to be put on the basis of the evaluations (steps S600 to S647) of the barometers SB, WB, BB, and TB of the subject (see Tables 2 to 5). In step S648, the PC 7 deduces the stride of the subject on the basis of the barometers WB and TB of the subject (see Table 6).

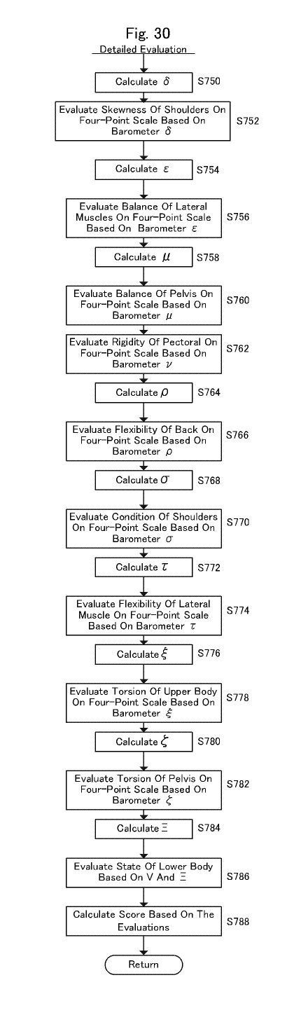

In step S650, the PC 7 performs a detailed evaluation process as shown in FIG. 30 as described below. In step S652, the PC 7 generates the human body image corresponding to the posture as determined in step S1000, the parts whose muscles are apt to go tight and the parts on which the fat is apt to be put as determined in step S648, and the stride as determined in step S649, and then displays the total result screen of FIG. 26 (FIG. 27) on the monitor 43.

The processes of steps S654, S662, S658, S656, and S660 are the same as the processes of steps S333, S341, S337, S335, and S339 of FIG. 16 respectively. However, the PC 7 outputs the printed result of FIG. 28 in step S662. Incidentally, the above constants C7 to C11 are positive integers.

FIG. 30 is a flow chart showing a detailed evaluation process in step S650 of FIG. 29. Referring to FIG. 30, in step S750, the PC 7 calculates the barometer δ on the basis of the movable range A of the right arm and the movable range B of the left arm in performing the first motion. In step S752, the PC 7 evaluates the skewness of the shoulders on four-point scale on the basis of the barometer δ.

The comment to be displayed in the frame 120 (FIG. 28) when determining that the skewness of the shoulders is normal, and the comment to be displayed in the frame 120 (FIG. 28) when determining that the skewness of the shoulders is bad (abnormal) are the same as those when determining the normal and the abnormal in the above embodiment respectively. If it is determined that the condition of the shoulders is good, for example, the comment “The balance of the right and left shoulders is good.” is displayed in the frame 120. Also, if it is determined that the skewness of the shoulders is singularly bad (worst), for example, the comment “The balance of the right and left shoulders is considerably lost, and the right (left) shoulder is considerably high. Don't you usually hold a bag only by any one of hands? If the condition where the balance of the right and left is lost is continued, various symptoms such as the shoulder stiffness, the migraine, and the eyestrain may be caused.” is displayed in the frame 120.

In step S754, the PC 7 calculates the barometer ε on the basis of the displacement width RR of the upper body in the right direction, the displacement width O of the lumbar part in the right direction, the displacement width LL of the upper body in the left direction, and the displacement width S of the lumbar part in the left direction when performing the third motion. In step S756, the PC 7 evaluates the balance of sides of a body (lateral muscles) on four-point scale on the basis of the barometer ε.

The comment to be displayed in the frame 112 (FIG. 28) when determining that the balance of sides of a body is normal, and the comment to be displayed in the frame 112 (FIG. 28) when determining that the balance of sides of a body is bad (abnormal) are the same as those when determining the normal and the abnormal in the above embodiment respectively. If it is determined that the balance of sides of a body is good, for example, the comment “The balance of the waist is good. Have the daily lives where the right and left of the upper body are balanced like that.” is displayed in the frame 112. Also, if it is determined that the balance of sides of a body is singularly bad (worst), for example, the comment “The balance of the right and left of the waist is considerably lost. The muscle from the right (left) side of the waist to the back is apt to be used, conversely the muscle of the left (right) side is hard to be used. The discomfort of the lumbar part, the backache, the difference of the waist lines, and so on may occur.” is displayed in the frame 112.

In step S758, the PC 7 calculates the barometer μ on the basis of the displacement width Θ of the lumbar part in the left direction, and the displacement width Σ of the lumbar part in the right direction when performing the sixth motion. In step S760, the PC 7 evaluates the right/left balance of the pelvis on four-point scale on the basis of the barometer p. The comment to be displayed in the frame 126 (FIG. 28) when determining that the right/left balance of the pelvis is normal, and the comment to be displayed in the frame 126 (FIG. 28) when determining that the right/left balance of the pelvis is bad (abnormal) are the same as those when determining the normal and the abnormal in the above embodiment respectively. If it is determined that the right/left balance of the pelvis is good, for example, the comment “The right/left balance of the pelvis is good. Keep the condition of the balanced pelvis continuously.” is displayed in the frame 126. Also, if it is determined that the right/left balance of the pelvis is singularly bad (worst), for example, the comment “Your condition is the condition where the pelvis is apt to be displaced to the right (left). The hips are apt to swing horizontally when walking, and the pelvis is apt to be displaced to the right (left) when standing. If you continue this daily lives, the backache may be caused, and the shoulder stiffness, the headache, and so on may be caused because of the skewness of the backbone.” is displayed in the frame 126.

In step S762, the PC 7 acquires the displacement width D of the upper body in the back direction when performing the first motion as the barometer v, and then evaluates the rigidity of the pectoral on four-point scale on the basis of the barometer v.

The comment to be displayed in the frame 110 (FIG. 28) when determining that the rigidity of the pectoral is normal, and the comment to be displayed in the frame 110 (FIG. 28) when determining that the rigidity of the pectoral is bad (abnormal) are the same as those when determining the normal and the abnormal in the above embodiment respectively. If it is determined that the rigidity of the pectoral is good, for example, the comment “The flexibility of the muscle of the breast is good. Stretch routinely so as to keep the flexibility.” is displayed in the frame 110. Also, if it is determined that the rigidity of the pectoral is singularly bad (worst), for example, the comment “The muscle of the breast is considerably tight. Since this causes the shoulder stiffness, the frozen shoulder, and so on as well as the stoop if it is left, secure the flexibility steadily.” is displayed in the frame 110.

In step S764, the PC 7 calculates the barometer ρ on the basis of the movable range U of the upper body, and the displacement width [symbol] of the lumbar part in the front direction when performing the fourth motion. In step S766, the PC 7 evaluates the flexibility of the muscle of the back on four-point scale on the basis of the barometer p.

The comment to be displayed in the frame 124 (FIG. 28) when determining that the flexibility of the muscle of the back (the roundness of the back) is normal, and the comment to be displayed in the frame 124 (FIG. 28) when determining that the flexibility of the muscle of the back (the roundness of the back) is bad (abnormal) are the same as those when determining the normal and the abnormal in the above embodiment respectively. If it is determined that the flexibility of the muscle of the back is good, for example, the comment “The flexibility of the back (backbone) is good.” is displayed in the frame 124. Also, if it is determined that the flexibility of the muscle of the back is singularly bad (worst), for example, the comment “The flexibility of the muscle of the back (backbone) lacks considerably. The lower back is apt to warp, you have the condition in which the tiredness is apt to be accumulate due to the strain of the muscle of the lumbar pat, and therefore it is highly possible that the backache is caused.” is displayed in the frame 124.

In step S768, the PC 7 calculates the barometer σ on the basis of the displacement width E of the upper body in the left direction, and the displacement width F of the upper body in the right direction when performing the first motion. In step S770, the PC 7 evaluates the condition of the shoulders on four-point scale on the basis of the barometer σ.

The comment to be displayed in the frame 122 (FIG. 28) when determining that the shoulders are normal, and the comment to be displayed in the frame 122 (FIG. 28) when determining that the shoulders are bad (abnormal) are the same as those when determining the normal and the abnormal in the above embodiment respectively. If it is determined that the shoulders are good, for example, the comment “Both of the bladebones and the arms function normally.” is displayed in the frame 122. Also, if it is determined that the shoulders are singularly bad (worst), for example, the comment “The body considerably inclines leftward (rightward) due to nurse the right (left) arm. This may cause the failure of the right shoulder, the shoulder stiffness, and further the backache.” is displayed in the frame 122.

In step S772, the PC 7 calculates the barometer τ on the basis of the displacement width RR of the upper body in the right direction, the displacement width O of the lumbar part in the right direction, the displacement width LL of the upper body in the left direction, and the displacement width S of the lumbar part in the left direction when performing the third motion. In step S774, the PC 7 evaluates the flexibility of the lateral muscle on four-point scale on the basis of the barometer r.

The comment to be displayed in the frame 114 (FIG. 28) when determining that the flexibility of the lateral muscle is normal, and the comment to be displayed in the frame 114 (FIG. 28) when determining that the flexibility of the lateral muscle is bad (abnormal) are the same as those when determining the normal and the abnormal in the above embodiment respectively. If it is determined that the flexibility of the lateral muscle is good, for example, the comment “The flexibility of sides of the body is good.” is displayed in the frame 114. Also, if it is determined that the flexibility of the lateral muscle is singularly bad (worst), for example, the comment “The flexibility of sides of the body lacks considerably. The muscle from the flank to the back is apt to go tight, and this may cause the backache. Also, the extension of a rib is apt to be small, and this is a bad influence on the deep breath.” is displayed in the frame 114.

In step S776, the PC 7 calculates the barometer ξ on the basis of the displacement widths α and γ of the upper body in the front direction when performing the third motion. In step S778, the PC 7 evaluates the right/left torsion of the upper body on four-point scale on the basis of the barometer ξ.

The comment to be displayed in the frame 116 (FIG. 28) when determining that the right/left torsion of the upper body is within the normal range, and the comment to be displayed in the frame 116 (FIG. 28) when determining that the right/left torsion of the upper body is bad (abnormal) are the same as those when determining the normal and the abnormal in the above embodiment respectively. If it is determined that the upper body is good, for example, the comment “There is no right/left torsion of the upper body. Keep the balanced condition like that.” is displayed in the frame 116. Also, if it is determined that the right/left torsion of the upper body is singularly bad (worst), for example, the comment “The right/left torsion of the upper body appears prominently. Doesn't the right (left) shoulder twist forward? You tend to twist the body leftward (rightward). You often twist leftward (rightward) in the daily lives, and this causes the difference between the right waist line and the left waist line, the backache, and so on.” is displayed in the frame 116.

In step S780, the PC 7 calculates the barometer ζ on the basis of the displacement width [symbol] of the lumbar part in the front direction and the displacement width [symbol] of the lumbar part in the back direction when performing the sixth motion. In step S782, the PC 7 evaluates the torsion of the pelvis on four-point scale on the basis of the barometer ζ.

The comment to be displayed in the frame 128 (FIG. 28) when determining that the torsion of the pelvis is within the normal range, and the comment to be displayed in the frame 128 (FIG. 28) when determining that the torsion of the pelvis is bad (abnormal) are the same as those when determining the normal and the abnormal in the above embodiment respectively. If it is determined that the pelvis is good, for example, the comment “The pelvis hardly twists.” is displayed in the frame 128. Also, if it is determined that the torsion of the pelvis is singularly bad (worst), for example, the comment “The pelvis considerably twists. The left (right) pelvis is inclined in the more back direction. The right (left) stride may be larger than the left (right) stride.” is displayed in the frame 128.

Incidentally, in the above examples of the contents of the comments, there is the part where either “right” or “left” is chosen to use (the alternative being provided in parentheses). In this case, either “right” or “left” is chosen in accordance with the posture of the subject as evaluated.

In step S784, the PC 7 calculates the parameter Ξ on the basis of the displacement width [symbol] of the lumbar part in the front direction and the displacement width [symbol] of the lumbar part in the back direction when performing the sixth motion. In step S786, the PC 7 evaluates the condition (any one of the conditions 1 to 6) of the lower body on the basis of the parameter Ξ and the displacement width [symbol] of the lumbar part in the front direction when performing the fourth motion. In this case, the comment (text) is the same as that of the embodiment.

In step S788, the PC 7 calculates the score BP on the basis of the above evaluations (S752, S756, S760, S762, S766, S770, S774, S778, S782, and S786) so as to display the score displaying section 54.

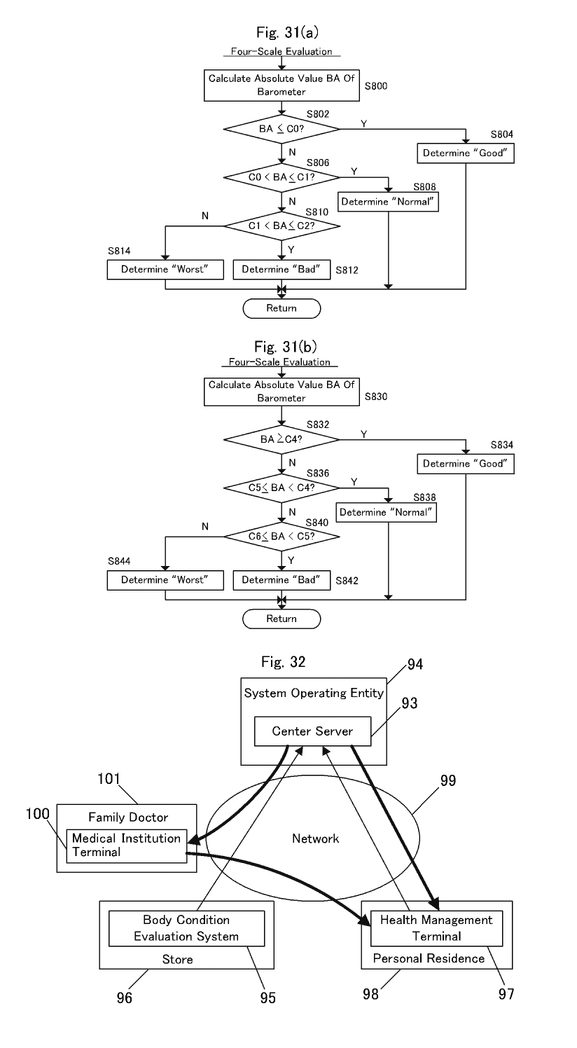

FIG. 31(a) is a flow chart showing a four-scale evaluation process in steps S752, S756, S760, S762, S770, S778, and S782 of FIG. 30. Referring to FIG. 31(a), in step S800, the PC 7 calculates the absolute value BA of the barometer. In step S802, the PC 7 determines whether or not the absolute value BA is not more than the constant C0, the process proceeds to step S804 if it is not more than the constant C0, otherwise the process proceeds to step S806. In step S804, the PC 7 determines the evaluation “Good”.

In step S806, the PC 7 determines whether or not the absolute value BA exceeds the constant C0 and furthermore is not more than the constant C1, the process proceeds to step S808 if it is within the range, otherwise the process proceeds to step S810. In step S808, the PC 7 determines the evaluation “Normal”.

In step S810, the PC 7 determines whether or not the absolute value BA exceeds the constant C1 and furthermore is not more than the constant C2, the process proceeds to step S812 if it is within the range, otherwise the process proceeds to step S814. In step S812, the PC 7 determines the evaluation “Bad”. On the other hand, in step S814, the PC 7 determines the evaluation “Worst”.

In this case, constants C0 to C2 (positive integers) are given for each of barometers δ, ε, μ, v, σ, ξ, and ζ through an experiment and a trial and error process.

FIG. 31(b) is a flow chart showing the four-scale evaluation process in steps S766 and S774 of FIG. 30. Referring to FIG. 31(b), in step S830, the PC 7 calculates the absolute value BA of the barometer. In step S832, the PC 7 determines whether or not the absolute value BA is not less than the constant C4, the process proceeds to step S834 if it is not less than the constant C4, otherwise the process proceeds to step S836. In step S834, the PC 7 determines the evaluation “Good”.

In step S836, the PC 7 determines whether or not the absolute value BA is not less than the constant C5 and furthermore is below the constant C4, the process proceeds to step S838 if it is within the range, otherwise the process proceeds to step S840. In step S838, the PC 7 determines the evaluation “Normal”.

In step S840, the PC 7 determines whether or not the absolute value BA is not less than the constant C6 and furthermore is below the constant C5, the process proceeds to step S842 if it is within the range, otherwise the process proceeds to step S844. In step S842, the PC 7 determines the evaluation “Bad”. On the other hand, in step S844, the PC 7 determines the evaluation “Worst”.

In this case, constants C4 to C6 (positive integers) are given for each of barometers ρ and τ through an experiment and a trial and error process.

By the way, in the first modification, the hardware thereof is the same as that of the above embodiment, and the posture of the subject is evaluated on the basis of the parameters and the barometers similar to the above embodiment. Accordingly, the first modification has the advantage similar to the above embodiment.

[Second Modification]

FIG. 32 is a view showing the entire configuration of a health management system in accordance with the second modification of the embodiment of the present invention. Referring to FIG. 32, the health management system includes a center server 93, the body condition evaluation system 95 according to the above first modification, a health management terminal 97, a medical institution terminal 100, and a network 99.

The center server 93 is administered by an operating entity 94 running the health management system. The body condition evaluation system 95 is set up in a store 96 such as a gym, a bathhouse, a practitioner office, an esthetic salon. The health management terminal 97 is set up in a personal residence 98 of a user (the subject of the above first modification). The medical institution terminal 100 is a terminal of a family doctor 101 of the user. The network 99 includes Internet, LAN, and so on.

A measurement and evaluation result, an exercise menu, and so on in the body condition evaluation system 95 are transmitted to the center server 93 through the network 99, and are stored in relation to a date for each user in a hard disk drive of the center server 93. Also, blood pressure, a weight, the number of steps, exercise information, and so on measured by the health management terminal 97 are transmitted to the center server 93 through the network 99, and are stored in relation to a date for each user in the hard disk drive of the center server 93.

The center server 93 manages the above information transmitted from the body condition evaluation system 95 and the health management terminal 97 (hereinafter referred to as “user total information”) in relation to a date for each user. The operating entity 94 has specialists of medical care and health such as a healthcare professional such as a doctor and a work therapist, a dietician, and a trainer. These specialists performs close investigation, analysis, and so on of the user total information, creates “health management information” (text, animation, static images, and sound) such as daily life guidance (including encouragement and so on) and an exercise menu suitable for the user, and the center server 93 transmits it to the health management terminal 97 through the network 99. The user acquires the health management information via the health management terminal 97, and has daily lives and performs exercise in accordance therewith. Also, the user accesses the center server 98 via the health management terminal 97, and can read his/her own information (the measured values, the evaluations, the exercise menu, and so on) transmitted from the body condition evaluation system 95. Incidentally, the information of the user transmitted from the body condition evaluation system 95 is also referred to as the “health management information”.

Also, the medical institution terminal 100 accesses the center server 93, and can display or acquire the user total information. The family doctor 101 can perform a physical examination and make diagnoses on the basis of the condition and the user total information of the user which has visited for the purpose of diagnosis or therapy. Also, the family doctor 101 creates “medical information” (text, animation, static images, and sound) such as daily life guidance (including encouragement and so on) and an exercise prescription suitable for the user on the basis of the diagnosis result and the user total information, and the medical institution terminal 100 transmits it to the health management terminal 97 through the network 99. The user acquires the medical information via the health management terminal 97, and has daily lives and performs exercise in accordance therewith.

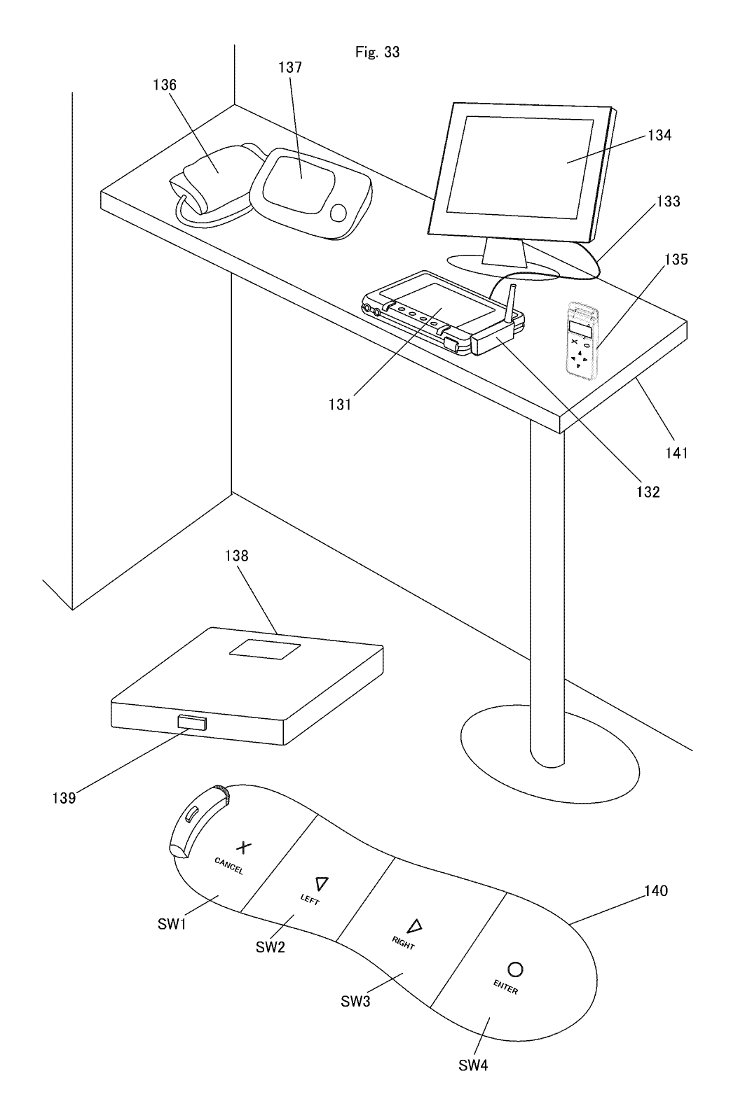

FIG. 33 is a view showing the entire configuration of the health management terminal 97 of FIG. 32. Referring to FIG. 33, the health management terminal 97 includes a computer 131, an antenna unit 132, a monitor 134, a pedometer 135, a weight scale 138, a mat-type controller 140 (hereinafter referred to as the “mat 140”), and a sphygmomanometer 137. The antenna unit 132 is attached to (electrically connected with) the computer 131 The monitor 134 is coupled with the computer 131 via a cable 133. Accordingly, a video signal VD and an audio signal AU generated by the computer 131 are given to the monitor 134 via the cable 133.

The pedometer 135 is carried by the user, and measures the daily number of steps to record. When a switch 139 is pushed, the weight scale 138 runs, and measures the weight of the user who gets thereon. The mat 140 includes four foot switches SW1 to SW4 which are aligned, and detects the stepping motion of the user. The sphygmomanometer 137 includes a cuff 136 which is warped around the arm, and measures the blood pressure of the user which warps the cuff 136 around the arm.

In the second modification, for example, the computer 131 to which the antenna unit 132 is attached, the monitor 134, and the sphygmomanometer 137 are placed on a board 141 in the personal residence 98. Also, the weight scale 138 and the mat 140 are placed on a floor of a room where the board 141 is installed.

Next, electric configurations of the respective devices will be described.

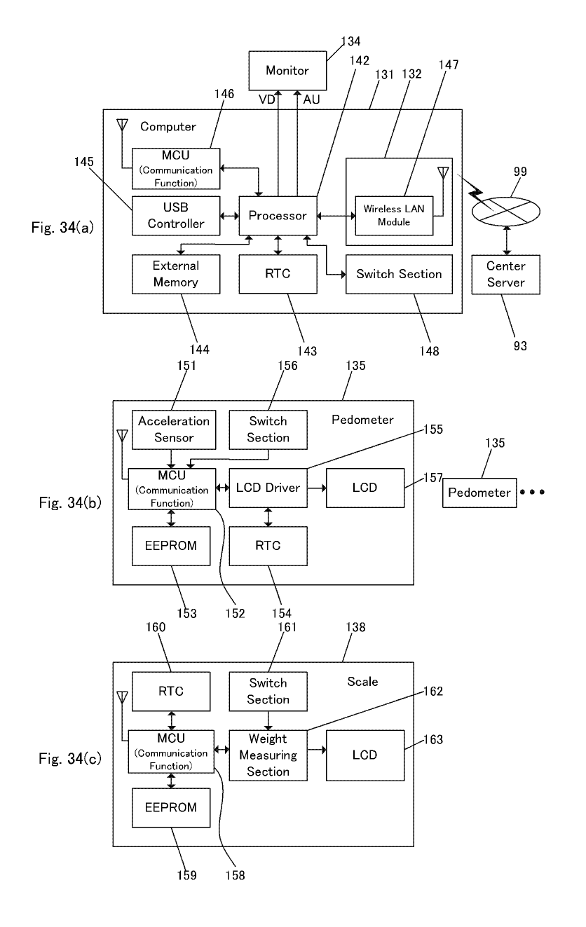

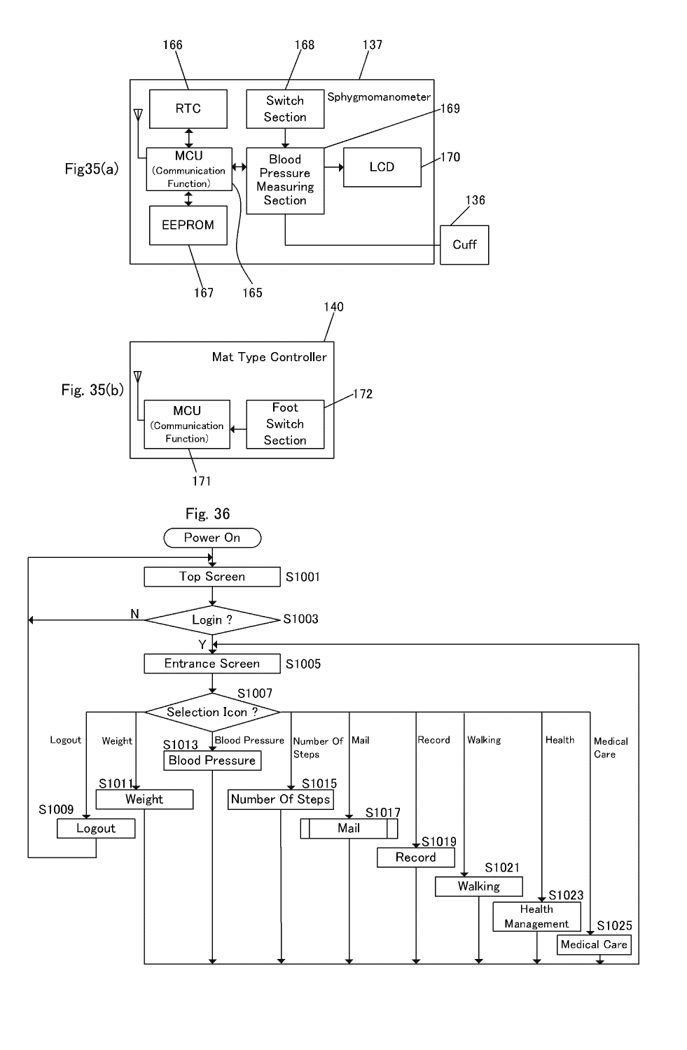

FIG. 34(a) is a view showing the electric configurations of the computer 131 and the antenna unit 132 of FIG. 33. FIG. 34(b) is a view showing the electric configurations of the pedometer 135 of FIG. 33. FIG. 34(c) is a view showing the electric configurations of the weight scale 138 of FIG. 33. FIG. 35(a) is a view showing the electric configuration of the sphygmomanometer 137 of FIG. 33. FIG. 35(b) is a view showing the electric configuration of the mat type controller 140 of FIG. 33.

Referring to FIG. 34(a), the computer 131 is provided with a switch section 148, a processor 142, an external memory 144, an MCU 146 with a wireless communication function, a USB controller 145, and RTC (Real Time Clock) 143. The operated signals of the switch section 148 are inputted to the processor 142. The switch section 148 includes a cancel key, an enter key, and arrow keys (not shown in the figure). The antenna unit 132 includes a wireless LAN module 147.

The processor 142 is connected with the external memory 144. The external memory 144 is provided with a ROM, a RAM, a flash memory, and so on in accordance with the specification of the system. The external memory 144 includes a program area, an image data area, and an audio data area. The program area stores control programs. The image data area stores all of the image data items which constitute the screens to be displayed on the monitor 134. The audio data area stores audio data for generating music, voice, sound effect, and so on. The processor 142 executes the control programs in the program area, reads the image data in the image data area and the audio data in the audio data area, processes them, generates a video signal VD and an audio signal AU, and then outputs them to the monitor 134.

Also, the processor 142 executes the control program, and instructs the MCU 146 to communicate with the node (the MCU 152 of the pedometer 135, the MCU 158 of the weight scale 138, the MCU 165 of the sphygmomanometer 137, and the MCU 171 of the mat 140) and acquire behavior information, body information, and motion information of the user. The MCU 146 receives an instruction from the processor 142, and then receives the behavior information, the body information, the motion information of the user from the node, demodulates them, and then sends them to the processor 142.

In the second modification, the “behavior information” is information of an exercise form (standard walking, rapid walking, or running) and the number of times for each exercise form (the number of steps). However, the “behavior information” may be information of an exercise form (a content of training such as circuit training and weight training, a content of sports such as tennis, a movement of each part of a body, or a content and form of the other body movement), the number of times for each exercise form (e.g., the number of times for each body movement such as the number of times of weightlift), start and end for each exercise form (e.g., start and end for each body movement such as the start and end of the play of the tennis), and the other information relating to behavior. Also, the “behavior information” may include daily activity information. The “daily activity information” includes contents of housework such as cleaning, washing, and cooking, and information of a meal (kinds, contents, calories, and so on), information of carry, information of work, information of a school, information of a work trip and move (including a ride on a conveyance such as a car, a bicycle, a motorcycle, an electric train, an airplane, and a ship), an avocation, and so on, information of the number of times of them, information of start and end of them, and information of the other behavior and activity which naturally occur in daily life of an individual.

Also, in the second modification, the “body information” is information of weight measured by the weight scale 138 and blood pressure measured by the sphygmomanometer 137. However, the “body information” may include body size information such as a height, an abdominal circumference and BMI, information of eyesight, information of intensity of daily activity, information of the inside of the body (information of urine, information of erythrocyte such as erythrocyte count, a body fat percentage, information of a hepatic function such as γ-GTP, information of fat metabolism such as HDL cholesterol and neutral fat, information of glucose metabolism such as a blood glucose value, a cardiac rate, and so on), and the other information representing condition of a body.

Also, in the second modification, the “motion information” is ON/OFF information of the foot switches SW1 to SW4. However, the “motion information” may include information representing the motion of user which is calculated on the basis of the acceleration data from the acceleration sensor, information representing the motion of user which is calculated by analyzing the image of the user captured by the camera, and the other information representing the motion of the user which the motion sensor detects.

BY the way, the processor 142 stores the behavior information, the body information, and the motion information as received in the external memory 144. Also, the processor 142 processes the behavior information and the body information into a graph, a table, or the like to display them on the monitor 134. Further, in the exercise mode, the processor 142 generates the video signal VD representing the interactive video in accordance with the motion information of the user from the mat 140.

Still further, the processor 142 executes the control program and instructs the wireless LAN module 147 to transmit the behavior information and the body information received from the node to the center server 93. The wireless LAN module 147 receives the instruction from the processor 142 and then transmits the behavior information and the body information of the user to the center server 93 through the network 99. Also, the wireless LAN module 147 can receive mail, the health management information, the medical information, and so on from the center server 93 through the network 99, and sends them to the processor 142. Then, the processor 142 stores the information transmitted by the center server 93 in the external memory 144, and displays them on the monitor 134 in response to the operation of the user.

The USB controller 145 is for connecting to a USB device such as a personal computer, and transfers the behavior information and the body information stored in the external memory 144 to the USB device under the control of the processor 142. The RTC 143 generates time information and sends it to the processor 142. The processor 142 displays the time information on the monitor 134 as necessary.

The internal configuration of the processor 142 will be described simply. Although not shown in the figure, the processor 142 is provided with a central processing unit (hereinafter referred to as the “CPU”), a graphics processing unit (hereinafter referred to as the “GPU”), a sound processing unit (hereinafter referred to as the “SPU”), a geometry engine (hereinafter referred to as the “GE”), an external interface block, a main RAM, an A/D converter (hereinafter referred to as the “ADC”) and so forth.

The CPU performs various operations and controls the entire system by executing the programs stored in the external memory 144. The CPU performs the process relating to graphics operations, which are performed by running the program stored in the external memory 144, such as the calculation of the parameters required for the expansion, reduction, rotation and/or parallel displacement of the respective objects and the calculation of eye coordinates (camera coordinates) and view vector. In this description, the term “object” is used to indicate a unit which is composed of one or more polygons or sprites and to which expansion, reduction, rotation and parallel displacement transformations are applied in an integral manner.

The GPU serves to generate a three-dimensional image composed of polygons and sprites on a real time base, and converts it into the video signal VD. The SPU generates the audio signal AU. The GE performs geometry operations for displaying a three-dimensional image. Specifically, the GE executes arithmetic operations such as matrix multiplications, vector affine transformations, vector orthogonal transformations, perspective projection transformations, the calculations of vertex brightnesses/polygon brightnesses (vector inner products), and polygon back face culling processes (vector cross products).

The external interface block is an interface with peripheral devices (the MCU 146, the USB controller 145, the RTC 143, the wireless LAN module 147, and the switch section 148 in the second modification). The ADC serves to convert an analog signal, which is input from an analog input device, into a digital signal. The main RAM is used by the CPU as a work area, a variable storing area, a virtual memory system management area and so forth.

Referring to FIG. 34(b), the pedometer 135 is provided with an MCU 152 with a wireless communication function, an EEPROM 153, an acceleration sensor 151, an LCD driver 155, an LCD 157, an RTC 154, and a switch section 156. The switch section 156 includes a decision button and arrow keys (not shown in the figure). The acceleration sensor 151 detects accelerations ax, ay, and az in the respective direction of the three axes (x, y, z) which are at right angles to one another.

In the pedometer mode, the MCU 152 measures the number of steps of the user on the basis of the acceleration data from the acceleration sensor 151, stores it in the EEPROM 153 in association with the time information (including a date) generated by the RTC 154, and sends it to the LCD driver 155. The LCD driver 155 displays the received data of the number of the steps on the LCD 157.

Also, in the pedometer mode, the MCU 152 controls the LCD driver 155 in response to the operation of the decision button, and thereby changes the display of the LCD 157. Further, in the pedometer mode, the MCU 152 shifts to the communication mode when the decision button and the cancel button are simultaneously pressed. In the communication mode, the MCU 152 transmits the data of the number of steps as stored in the EEPROM 153 in the pedometer mode to the MCU 146. The LCD driver 155 receives the time information from the RTC 154, and displays it on the LCD 157. The RTC 154 generates the time information.

The plurality of the pedometers 135 each of which has such configuration are prepared, and distributed to the plurality of the users (members of a family in the second modification). Incidentally, unique identification information (a node ID as described below) is assigned to the each pedometer 135, and is stored therein.

Referring to FIG. 34(c), the weight scale 138 is provided with a weight measuring section 162, an MCU 158 with a wireless communication function, an EEPROM 159, an RTC 160, a switch section 161, and an LCD 163. The switch section 161 includes the switch 139 of FIG. 33. In response to the push of the switch 139, the weight measuring section 162 measures the weight of the user which gets on the weight scale 138, converts it into the digital data to send to the MCU 158, and displays the weight value on the LCD 157. The MCU 158 stores the received weight value in the EEPROM 153. In this case, the MCU 158 stores the weight value in association with the time information (including the date) generated by the RTC 160. Also, the MCU 152 transmits the weight value associated with the time information as stored in the EEPROM 159 to the MCU 146 of the computer 131.

Referring to FIG. 35(a), the sphygmomanometer 137 is provided with a blood pressure measuring section 169, an MCU 165 with a wireless communication function, an EEPROM 167, an RTC 166, a switch section 168, and an LCD 170. In response to the push of a specified switch (not shown in the figure) of the switch section 168, the blood pressure measuring section 169 measures the blood pressure of the user which warps the cuff 136 around the arm, converts it into the digital data to send to the MCU 165, and displays the blood pressure values on the LCD 170. The MCU 165 stores the received blood pressure values in the EEPROM 167. In this case, the MCU 165 stores the blood pressure values in association with the time information (including the date) generated by the RTC 166. Also, the MCU 165 transmits the blood pressure values associated with the time information as stored in the EEPROM 167 to the MCU 146 of the computer 131.

Referring to FIG. 35(b), the mat 140 is provided with an MCU 171 with a wireless communication function, and a foot switch section 172. The foot switch section 172 includes the foot switches SW1 to SW4. In the exercise mode, the MCU 171 transmits the ON/OFF information of the foot switches SW1 to SW4 at fixed time intervals to the MCU 146 of the computer 131.

Next, the flow of the processing will be described using the flowcharts.

FIG. 36 is a flowchart showing the overall process flow by the processor 142 of FIG. 34(a). Referring to FIG. 36, in step S1001, the processor 142 displays a top screen on the monitor 134. When the user performs a prescribed operation to the pedometer 135, the pedometer 135 transmits the identification information thereof to the processor 142. In step S1003, the processor 142 determines whether or not the identification information is received from the pedometer 135, the process returns to step S1001 if it is not received, conversely the process permits the user of the pedometer 135 to login and proceeds to step S1005 if it is received. In step S1005, the processor 142 displays an entrance screen for the user associated with the received identification information on the monitor 134. For example, the entrance screen contains a health icon, a medical icon, a weight icon, a blood pressure icon, a step number icon, a mail icon, a record icon, a walking icon, a logout icon, a selection icon, and a decision icon.

The user selects the intended icon displayed on the entrance screen by operating the cancel key, the enter key, and the arrow keys of the computer 131. In step S1007, the processor 142 receives the ON/OFF information from these keys, determines which of the icons is selected, and performs the processing corresponding thereto.

That is, the processor 142 proceeds to step S1009 if the logout icon is selected. In step S1009, the processor 142 performs the logouting processing, and then proceeds to step S1001 to display the top screen. The processor 142 proceeds to step S1011 if the weight icon is selected. In step S1011, the processor 142 graphs the weight value stored in the external memory 144 (e.g., a line graph) to display on the monitor 134. Then, the process returns to step S1005 in response to the push of the cancel key of the computer 131 to display the entrance screen.

For example, the three kinds of graphs, which have display with a one hour scale, display with a one day scale, and display with a one week scale with respect to the weight value respectively, are created as the graph to be displayed in step S1011, and each of them is selectively displayed in accordance with the key operation of the computer 131 of the user. In the display with the one hour scale, a horizontal axis is a time axis which is expressed in units of one hour (for one day) while the vertical axis indicates the weight value. Also, a cursor is displayed on the graph, and moves in the horizontal direction in units of one hour of the horizontal axis in accordance with the key operation of the computer 131. Then, the weight value and the BMI (Body Mass Index) at the time indicated by the cursor are displayed in the lower area of the screen. In the display with the one day scale, a horizontal axis is a time axis which is expressed in units of one day (for one week) while the vertical axis indicates the weight value. Also, a cursor is displayed on the graph, and moves in the horizontal direction in units of one day of the horizontal axis in accordance with the key operation of the computer 131. Then, the weight value and the BMI at the day indicated by the cursor are displayed in the lower area of the screen. In the display with the one week scale, a horizontal axis is a time axis which is expressed in units of one day (for one month) while the vertical axis indicates the weight value. Also, a cursor is displayed on the graph, and moves in the horizontal direction in units of one week of the horizontal axis in accordance with the key operation of the computer 131. Then, the average of the weight values and the average of the BMIs in the week indicated by the cursor are displayed in the lower area of the screen.

By the way, if the processor determines that the blood pressure icon is selected in step S1007, the process proceeds to step S1013. In step S1013, the processor 142 graphs the blood pressure values (a diastolic blood pressure and a systolic blood pressure) stored in the external memory 144 (e.g., a line graph) to display on the monitor 134. Then, the process returns to step S1005 in response to the push of the cancel key of the computer 131 to display the entrance screen.

For example, the three kinds of graphs, which have display with a one hour scale, display with a one day scale, and display with a one week scale with respect to the blood pressure values respectively, are created as the graph to be displayed in step S1013, and each of them is selectively displayed in accordance with the key operation of the computer 131 of the user. In the display with the one hour scale, a horizontal axis is a time axis which is expressed in units of one hour (for one day) while the vertical axis indicates the blood pressure. Also, a cursor is displayed on the graph, and moves in the horizontal direction in units of one hour of the horizontal axis in accordance with the key operation of the computer 131. Then, the diastolic blood pressure and the systolic blood pressure at the time indicated by the cursor are displayed in the lower area of the screen. In the display with the one day scale, a horizontal axis is a time axis which is expressed in units of one day (for one week) while the vertical axis indicates the blood pressure. Also, a cursor is displayed on the graph, and moves in the horizontal direction in units of one day of the horizontal axis in accordance with the key operation of the computer 131. Then, the diastolic blood pressure and the systolic blood pressure at the day indicated by the cursor are displayed in the lower area of the screen. In the display with the one week scale, a horizontal axis is a time axis which is expressed in units of one day (for one month) while the vertical axis indicates the blood pressure. Also, a cursor is displayed on the graph, and moves in the horizontal direction in units of one week of the horizontal axis in accordance with the key operation of the computer 131. Then, the average value of the diastolic blood pressures and the average value of the systolic blood pressures in the week indicated by the cursor are displayed in the lower area of the screen.

By the way, if the processor determines that the step number icon is selected in step S1007, the process proceeds to step S1015. In step S1015, the processor 142 graphs the number of steps stored in the external memory 144 (e.g., a bar graph) to display on the monitor 134. Then, the process returns to step S1005 in response to the push of the cancel key of the computer 131 to display the entrance screen.

For example, the three kinds of graphs, which have display with a one hour scale, display with a one day scale, and display with a one week scale with respect to the number of steps respectively, are created as the graph to be displayed in step S1013, and each of them is selectively displayed in accordance with the key operation of the computer 131 of the user. In this case, the number of steps is represented by a bar graph in separate colors for each exercise form (standard walking, rapid walking, or running). For example, respective bar in the bar graph are colored by three colors, and indicate the exercise forms.

In the display with the one hour scale, a horizontal axis is a time axis which is expressed in units of one hour (for one day) while the vertical axis indicates the number of steps. Also, a cursor is displayed on the graph, and moves in the horizontal direction in units of one hour of the horizontal axis in accordance with the key operation of the computer 131. Then, the number of steps and the calorie consumption at the time indicated by the cursor are displayed in the lower area of the screen. In the display with the one day scale, a horizontal axis is a time axis which is expressed in units of one day (for one week) while the vertical axis indicates the number of steps. Also, a cursor is displayed on the graph, and moves in the horizontal direction in units of one day of the horizontal axis in accordance with the key operation of the computer 131. Then, the number of steps and the calorie consumption at the day indicated by the cursor are displayed in the lower area of the screen. In the display with the one week scale, a horizontal axis is a time axis which is expressed in units of one day (for one month) while the vertical axis indicates the number of steps. Also, a cursor is displayed on the graph, and moves in the horizontal direction in units of one week of the horizontal axis in accordance with the key operation of the computer 131. Then, the number of steps and the calorie consumption in the week indicated by the cursor are displayed in the lower area of the screen.

By the way, if the processor determines that the mail icon is selected in step S1007, the process proceeds to step S1017. In step S1017, the processor 142 reads out the mail list stored in the external memory 144 and displays it on the monitor 134. Then, the content of the mail selected from the mail list in accordance with the key operation of the computer 131 is displayed on the monitor 134. Also, the process returns to step S1005 in response to the push of the cancel key of the computer 131 to display the entrance screen. Incidentally, the detail of the step S1017 will be described below.

Also, if the processor determines that the record icon is selected in step S1007, the process proceeds to step S1019. In step S1019, the processor 142 displays a calendar on the monitor 134. Then, the weight, the blood pressure, and the number of steps on a day selected from the calendar are read out from the external memory 144 in accordance with the key operation of the computer 131, and are displayed on the monitor 134. Also, the process returns to step S1005 in response to the push of the cancel key of the computer 131 to display the entrance screen.

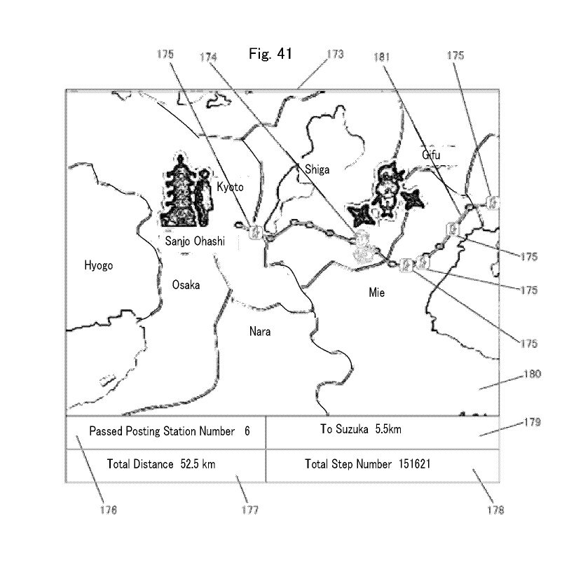

Also, if the processor determines that the walking icon is selected in step S1007, the process proceeds to step S1021. In step S1021, the processor 142 displays a map screen 173 shown in FIG. 41 on the monitor 134. Then, the process returns to step S1005 in response to the push of the cancel key of the computer 131 to display the entrance screen.

Referring to FIG. 41, the map screen 173 contains a map 180, a passed posting station displaying section 176 which indicates the number of passed posting stations along the way, a next posting station displaying section 179 which displays the distance to the next posting station, a total distance displaying section 177 which displays the total travel distance from the starting point, and the total step number displaying section 178 which displays the total number of steps from the starting point. The map 180 includes a route 181 on which the location of each posting station is shown. Icons 174 and 175 are displayed on the route 181. The icon 174 indicates the current location of the user logining. The icons 175 indicate the current locations of the other users. However, the other users are indicated under anonymity, and the content which identifies the individual user is not displayed. Incidentally, for example, the processor 142 has stride data as a predetermined value, and calculates the travel distance of the user by multiplying it by the number of steps.

Returning to FIG. 36, if the processor determines that the health icon is selected in step S1007, the process proceeds to step S1023. In step S1023, the processor 142 acquires the health management information (the daily life guidance and the exercise menu created by the specialist on the basis of the information from the body condition evaluation system 95, and the measurement and evaluation result and so on in the body condition evaluation system 95) stored in the center server 93 in accordance with the operation of the user, and then displays it on the monitor 134. Then, the process returns to step S1005 in response to the push of the cancel key of the computer 131 to display the entrance screen.

Also, if the processor determines that the medical icon is selected in step S1007, the process proceeds to step S1025. In step S1025, the processor 142 acquires the medical information (the daily life guidance, the exercise prescription, and so on created by the family doctor 101 on the basis of the diagnosis result and the user total information) stored in the center server 93 in accordance with the operation of the user, and then displays it on the monitor 1343. Then, the process returns to step S1005 in response to the push of the cancel key of the computer 131 to display the entrance screen.

As described above, since the present system displays the various information items of each user on the monitor 134, the present system may be called an information displaying system or an information displaying apparatus.

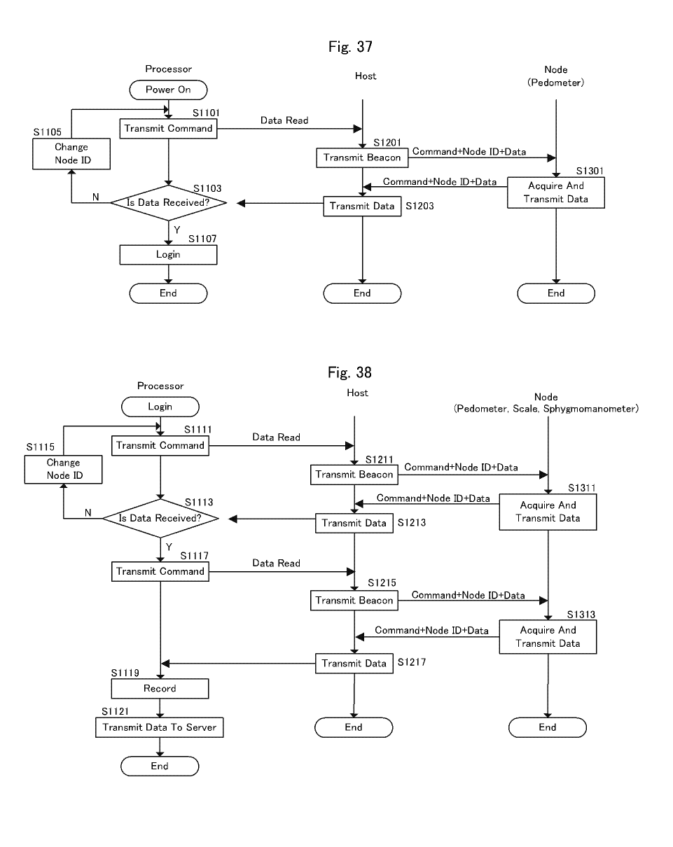

By the way, FIG. 37 is a view showing the communication procedure among the processor 142 of FIG. 34(a), the MCU (hereinafter referred to as a “host” in the explanation of this FIG. 146 of FIG. 34(a), and the node (the MCU 152 of the pedometer 135) of FIG. 34(b) (the login procedure).

Referring to FIG. 37, in step s1101, the processor 142 sends a read command of data, a node ID, and data to the host 146. Then, in step S1201, the host 146 transmits a beacon, which includes the read command, the node ID, and the data, to the node 152. In this case, the node ID is the information for identifying the node 152, i.e., the pedometer 135. In the second modification, for example, the respective 10 pedometers 135 to which the different node IDs are assigned can login.

When the node 152 receives the beacon including the node ID which is assigned to itself, the node 152 transmits the command received from the host 146, its own node ID, and the data requested by the command to the host 146 in step S1301.

In step S1203, the host 146 transmits the data (including the node ID) received from the node 152 to the processor 142. In step S1103, the processor 142 determines whether or not the data is received from the host 146, the process proceeds to step S1105 if it is not received, conversely the process proceeds to step S1107 if it is received.

In step S1105, the processor 142 changes the node ID to be included in the beacon, and then proceeds to step S1101. If the node 152 which has the node ID included in the beacon is not found, the response is not returned, and therefore another node is found by changing the node ID in step S1105. In this case, the processor 152 finds the node 152 cyclically in series from among the 10 nodes 152 until the node 152 is found. On the other hand, in step S1107, the processor 142 permits the user associated with the received node ID to login because the node 152 is found, and displays the entrance screen for the user (step S1005 of FIG. 36).

By the way, FIG. 38 is a view showing the communication procedure among the processor 142 of FIG. 34(a), the MCU (hereinafter referred to as a “host” in the explanation of this FIG. 146 of FIG. 34(a), and the node (the MCU 152 of the pedometer 135 of FIG. 34(b), the MCU 158 of the weight scale 138 of FIG. 34(c), or the MCU 165 of the sphygmomanometer 137 of FIG. 35(a)) (the data transfer procedure).

Referring to FIG. 38, in step S1111 after logining, the processor 142 sends a read command of data, a node ID, and data to the host 146. Then, in step S1211, the host 146 transmits a beacon, which includes the read command, the node ID, and the data, to the node. In this case, the node ID is the identification information assigned to each node, i.e., each of the pedometer 135, the weight scale 138, and the sphygmomanometer 137 which have logined.

When the node receives the beacon including the node ID which is assigned to itself, the node transmits the command received from the host 146, its own node ID, and the data requested by the command to the host 146 in step S1311.

In step S1213, the host 146 transmits the data (including the node ID) received from the node to the processor 142. In step S1113, the processor 142 determines whether or not the data is received from the host 146, the process proceeds to step S1115 if it is not received, conversely the process proceeds to step S1117 if it is received. In step S1115, the processor 142 changes the node ID to be included in the beacon, and then proceeds to step S1111. If the node which has the node ID included in the beacon is not found, the response is not returned, and therefore another node is found by changing the node ID in step S1115. In this case, the processor 152 finds the node cyclically in series from among the three nodes (the pedometer 135, the weight scale 138, and the sphygmomanometer 137 which have logined) until the node is found. On the other hand, if the node is found in step S1113, in step S1117 and the succeeding processes, the communication is performed with the found node.

In what follows, the case where the pedometer 135 is detected as the node in step S1113 will be described as an example.

In step s1117, the processor 142 sends a read command of data of the number of steps, a node ID of the pedometer 135, and data to the host 146. Then, in step S1215, the host 146 transmits a beacon, which includes the read command, the node ID of the pedometer 135, and the data, to the node 152.

Accordingly, the node 152 receives the beacon including the node ID assigned to itself. Thus, in step S1313, the node 152 acquires the data of the number of steps associated with the time information (including the date) from the EEPROM 153, and then transmits it together with its own node ID and the received command to the host 146.

In step S1217, the host 146 transmits the data (including the number of steps and the node ID) received from the node 152 to the processor 142. Then, in step S1119, the processor 142 associates the data of the number of steps as received with the node ID (or the user ID) to store in the external memory 144. And, in step S1121, the data of the number of steps as received is transmitted along with the node ID (or the user ID) to the center server 93 through the wireless LAN module 147 and the network 99. Thereupon, the center server 93 associates the received data of the number of steps with the received node ID (or the user ID) to store in the hard disk drive.

Incidentally, in the case where the weight scale 138 is detected as the node in step S1113, in the description of step S1117 and the succeeding processes, the node (pedometer) 152 is replaced by the node (weight scale) 158, the number of steps is replaced by the weight, and the EEPROM 153 is replaced by the EEPROM 159. Also, in the case where the sphygmomanometer 137 is detected as the node in step S1113, in the description of step S1117 and the succeeding processes, the node (pedometer) 152 is replaced by the node (sphygmomanometer) 165, the number of steps is replaced by the blood pressure, and the EEPROM 153 is replaced by the EEPROM 167.

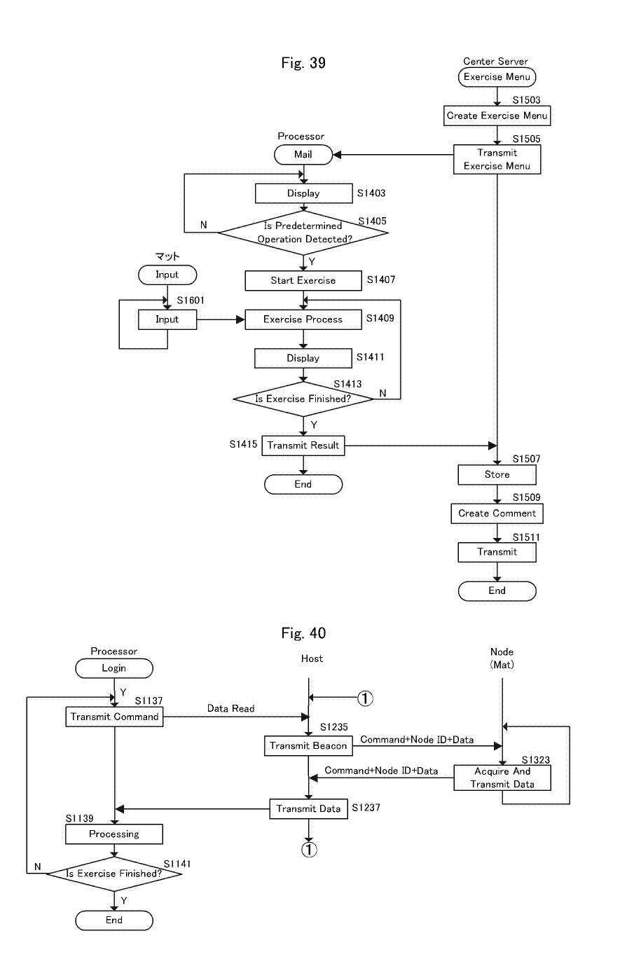

By the way, FIG. 39 is a flowchart showing the mail process (the exercise menu acquisition process) in step S1017 of FIG. 36. Referring to FIG. 39, in step S1503, the center server 93 creates the exercise menu for each user in accordance with predetermined algorithm on the basis of the data (the records of the number of steps, the weight, and the blood pressure) transmitted by the body condition evaluation system 95 of FIG. 32 and the step S1121 of FIG. 38. In step S1505, the center server 93 transmits the exercise menu created for each user as an E-mail to the processor 142 through the network 99 and the wireless LAN module 147. Then, the processor 142 sorts the received E-mails by the user, and then stores for each user.

Incidentally, the processes of steps S1403 to S1415 indicate the detail of the mail process of step S1017 of FIG. 36. Accordingly, the stage is a stage where one user logins and then the mail icon is selected. Further, it is assumed that the user selects the mail including the exercise menu from the mail list. Accordingly, in step S1403, the processor 142 displays the contents of the selected mail, i.e., the exercise menu on the monitor 134. Also, the screen for displaying the exercise menu contains a predetermined operation instruction.

In step S1405, the processor 142 determines whether or not the predetermined operation instruction is carried out, the process returns to step S1403 if it is not carried out, conversely the process proceeds to step S1407 to enter the exercise mode if it is carried out. The predetermined operation instruction instructs the user to select the predetermined icon in the screen by operating the key of the computer 131.

Then, in step S1407, the processor 142 starts performing the exercise processing. In step S1409, the processor 142 generates the image (the video signal VD) for instructing the user to perform the motion (the stepping motion) on the mat 140. Then, in step S1411, the processor 142 gives the image to the monitor 134 to display it. The user performs the stepping motion on the mat 140 in response to the instruction displayed on the monitor 134. Then, in step S1601, the mat 140 transmits the ON/OFF information of the foot switches SW1 to SW4 to the processor 142. Thereupon, in step S1409, the processor 142 generates the interactive image (video signal VD) in accordance with the ON/OFF information of the foot switches SW1 to SW4 of the mat 140 as input. And, in step S1411, the processor 142 displays the image on the monitor 134. In step S1413, the processor 142 determines whether or not the exercise is finished, the process proceeds to step S1415 if it is finished, conversely the process returns to step S1409 if it is not finished. In step S1415, the processor 142 transmits the result of the exercise and the node ID (or the user ID) to the center server 93 through wireless LAN module 147 and the network 99.

By the way, in step S1507, the center server 93 receives the result of the exercise of the user and the node ID transmitted from the processor 142 to store them in the hard disk drive. In step S1509, the center server 93 creates the comment on the basis of the result of the exercise in accordance with predetermined algorithm. In step S1511, the center server 93 transmits the comment as an E-mail to the processor 142 through the network 99 and the wireless LAN module 147.

As described above, the exercise mode makes the user exercise. Accordingly, the present system may be called an exercise support system or an exercise support apparatus.

Next, the communication between the mat 140 and the processor 142 as shown in FIG. 39 will be described in a little more detail.

FIG. 40 is a view showing the communication procedure among the processor 142 of FIG. 34(a), the MCU 146 of FIG. 34(a), and the node (the mat 140 of FIG. 35(b)) (the data transfer procedure).

Referring to FIG. 40, in step S1137, the processor 142 sends the read command of the ON/OFF data of the foot switches SW1 to SW4, the node ID of the mat 140, and the data to the host 142. Then, in step S1235, the host 146 transmits the beacon including the read command, the node ID of the mat 140, and the data to the node 140.

Accordingly, the node 140 receives the beacon including the node ID assigned to itself. Thus, in step S1323, the node 140 acquires the ON/OFF data of the foot switches SW1 to SW4, and transmits it along with its own node ID and the received command to the host 146.

In step S1237, the host 146 transmits the data (including the ON/OFF data of the foot switches SW1 to SW4 and the node ID) received from the node 140 to the processor 142. Then, in step S1139, the processor 142 generates the interactive video image in accordance with the ON/OFF data of the foot switches SW1 to SW4 as received to display it on the monitor 134. The process of the step S1139 corresponds to the processes of the steps S1409 and S411 of FIG. 39. In step S1441, the processor 142 determines whether or not the exercise is finished, the processor 142 proceeds to step S1137 if it is not finished, conversely the processor 142 ends the process for acquiring the data if it is finished.

By the way, as described above, in the second modification, since the user total information is transmitted from the body condition evaluation system 95 and the health management terminal 97 to the center server 93, the specialists of medical care, health, and so on of the operating entity 94 can analyze the information and then give the appropriate health management information to the relevant user.

Also, the information of the posture of the subject as well as the body information such as the weight and the behavior information such as the number of steps are transmitted to the server 93. Thus, the specialists of medical care, health, and so on of the operating entity 94 of the center server 93 can more finely analyze and evaluate in comparison with the analysis and the evaluation based only on the body information and behavior information, and analyze and evaluate on the basis of the physical condition (posture) of the body. And, since the results of these analysis and evaluation are supplied the health management terminal 97, the subject can more finely carry out the health management based on the posture (the physical condition of the body).

Further, since the family doctor 101 can refer the daily body information, the daily behavior information, and the posture information of the subject as well as the condition of the subject (patient) at the hospital visiting, the family doctor 101 can more finely exactly diagnose and examine. It is generally believed that it is difficult for a doctor other than an orthopedic surgeon and so on which treat a disorder of a backbone, bones of extremities, a joint, and a muscle system to measure and acquire information of posture of a patient. In accordance with the second modification, even such doctor can easily acquire the information of the posture to utilize for the diagnosis and the creation of the exercise prescription. As a result, the subject can receive the daily life guidance and the exercise prescription based on more precise diagnosis from the family doctor 101 via the medical institution terminal 100 and the health management terminal 97.

Still further, not only are the measurement and the evaluation of the posture by the body condition evaluation system 95, and the measurement of the body information and so on by the health management terminal 97 performed, but the various information items for the health management are also continuously provided to the user from the medical institution terminal 100 and the health management terminal 97 thereafter, and therefore it is effectively possible to support the health management of the user.

Also, in accordance with the second modification, since the user logins by his/her own pedometer 135, the user naturally brings his/her own record of the behavior information (the number of steps) to the installation site of the interface. Consequently, it is possible to easily store the behavior information as well as the body information in the external memory 144 incidentally and manage. In this way, to record and manage the behavior information is the motive for performing the exercise such as walking necessary to cause the behavior information (the number of steps).

Further, in accordance with the second modification, since the exercise mode can be entered when the user views the exercise menu, it is possible to seamlessly link the check of the exercise menu and the performance of the exercise in accordance therewith. Consequently, it is possible to effectively support the continuation of the exercise by the user.

Meanwhile, the present invention is not limited to the above embodiments, and a variety of variations and modifications may be effected without departing from the spirit and scope thereof, as described in the following exemplary modifications.

(1) In the above description, the acceleration sensor is implemented in the sensor unit 3. However, in place thereof, an angular velocity sensor such as a gyroscope, a direction sensor, an inclination sensor, and so on may be implemented therein. Also, any combination of two or more of an acceleration sensor, an angular velocity sensor, a direction sensor (a geomagnetic sensor), and an inclination sensor may be implemented therein. Further, with regard to the respective sensors, one axis, two axes, or three axes is chosen and employed depending on the specification.

(2) In the above description, the movable range is obtained as the angle formed by the resultant vector R0# at the state of the start time of the motion and the resultant vector R1# at the state of the finish time of the motion (FIGS. 3(a) to 3(c)). That is, the movable range is an angle relative to the resultant vector R0#. However, the movable range may be defined as an angle formed by the Yw axis in the reference coordinate system and the resultant vector R1#. In this case, the movable range is an absolute angle because of fixity of Yw axis. Also, in that case, the state angle from the state of the start time of the motion to the state of the finish time of the motion is defined as an angle formed by the Yw axis and the resultant vector R.

Incidentally, the movable range and the state angle based on the resultant vector R0# may be called a relative movable range and a relative state angle respectively, and the movable range and the state angle based on the Yw axis may be called an absolute movable range and an absolute state angle respectively.

(3) In the above description, the displacement angle in the right-left direction is obtained as an angle formed by the Yw axis and the vector Rxy, and the displacement angle in the front-back direction is obtained as an angle formed by the Yw axis and the vector Rzy (FIGS. 3(a) to 3(c)). That is, the displacement angle is an angle relative to the Yw axis and furthermore the Yw axis is fixed, and therefore the displacement angle is an absolute angle. The displacement angle in the right-left direction may be defined as an angle formed by the vector Rxy at the state of the start time of the motion and the vector Rxy until the state of the finish time of the motion, and the displacement angle in the front-back direction may be defined as an angle formed by the vector Rzy at the state of the start time of the motion and the vector Rzy until the state of the finish time of the motion. In this case, the displacement width is a maximum value of the displacement angles from the state of the start time of the motion to the state of the finish time of the motion.

Incidentally, the displacement angle and the displacement width based on the Yw axis may be called an absolute displacement angle and an absolute displacement width respectively, and the displacement angles and the displacement widths based on the vectors Rxy and Rzy at the state of the start time of the motion may be called relative displacement angles and relative displacement widths respectively.

(4) In the above description, the relative movable range and the relative state angle, and the absolute displacement angle and the absolute displacement width are used as parameters for evaluating (see Table 1, and FIGS. 22 to 24). However, in place of them, the absolute movable range and the absolute state angle, and the relative displacement angle and the relative displacement width may be used as parameters for evaluating. Also, the relative movable range and the relative state angle, the absolute displacement angle and the absolute displacement width, the absolute movable range and the absolute state angle, and the relative displacement angle and the relative displacement width may be combined.

For example, in FIG. 22, the absolute movable range is used as the movable ranges of the right and left arms in the first motion in place of the relative movable range, the relative displacement width is used as the displacement widths of the upper body and the lower back in the first motion, the third motion, and the sixth motion in place of the absolute displacement width, and the others are not changed.

(5) As described above, the displacement angle displaying sections 105U1 and 105B show the displacement angles in real time by the cobweb chart (e.g., see FIG. 8). Specifically, the displacement angle in the right-left direction is plotted on the horizontal axis, the displacement angle in the front-back direction is plotted on the vertical axis, the four vertexes are joined, and the enclosed area is colored. In this case, in each direction, the maximum value of the displacement angle is the newest plotting dot. Thus, for example, with regard to the displacement angle in one direction, the maximum value is plotted at some point in time, if the displacement angle exceeding the maximum value is not detected thereafter, the plotting dot in the direction thereof is not updated. Accordingly, it is not possible to view the trajectory of the motion of the subject.

However, in addition thereto or separately, the trajectory of the motion of the subject may be displayed by plotting the displacement angle in the right-left direction as X coordinate (horizontal coordinate) and the displacement angle in the front-back direction as Y coordinate (vertical coordinate) on the XY plane in real time.

Also, it is also possible to evaluate the physical condition of the subject on the basis of such trajectory of the motion of the subject.

(6) In the above description, the subject is a human. However, the subject may be the other creature.

(7) In the above description, it is determined whether or not the displacement width CF exceeds 60 degrees (see Table 1). The value of 60 degrees is empirically given experimentally, and the value is not limited thereto.

(8) The sensor unit 3C may be mounted on the central line 2 of the subject 1 on the breast. Also, the sensor unit 3W may be mounted on the central line 2 of the subject 1 at the roughly position of a navel, i.e., on the belly near the pelvis.

(9) In the above description, the posture is classified into any one of 8 patterns (see Table 1). However, the number of classifications is not limited thereto. It is possible to increase the number of classifications by adding ranges with regard to the parameters AR, AL, CF, WR, and WL. An example is as follows.

It is determined that the shoulders incline upward to the left if AL−AR>q, are normal if −q≦AL−AR≦q, and incline upward to the right if AL−AR<−9. It is determined that the load is the right load if WL−WR>p, is normal if −p≦WL−WR≦p, and is the left load if WL−WR<−p. It is determined that the pelvis tilts forward if CF>r0, is normal if r1<CF≦r0, and tilts backward if CF≦r1. In this way, if three ranges are set for each of the shoulder, the load, and the pelvis, the posture can be classified into any one of 27 patterns. Incidentally, the constants q, p, r0, and r1 are defined through an experiment, a trial and error process, and so on.

(10) In the above second modification, the processor 142 acquires the data of the number of steps after logining. However, the processor 142 may acquire the data of the number of steps along with the node ID from the pedometer 135 when the processor 142 permits the login, and record them as the information of the user permitted the login in the external memory 144. In this way, since the data of the number of steps is transferred when logining, there is no need to perform the login and the transfer of the data of the number of steps separately, and therefore it is possible to improve the convenience of the user.

(11) The number of steps is displayed on the map in FIG. 41. However, the other behavior information may be displayed. Also, the body information may be displayed. Needless to say, both of the behavior information and the body information may be displayed. Further, needless to say, the way of the expression is not limited to the map.

(12) In the above second modification, the number of steps is used as the behavior information, and the pedometer 135 is employed as a device for measuring it. However, the portable device for measuring the behavior information is not limited thereto, and may be a device for measuring the other behavior information, or a device combined them. Also, in the above description, the weight and the blood pressure are used as the body information, and the weight scale 138 and the sphygmomanometer 137 are employed as devices for measuring them. However, the device for measuring the body information is not limited thereto, and may be a device for measuring the other body information, or a device combined them. Also, one type of device may be employed as the device for measuring the body information in place of the plural types of devices.

(13) In the above second modification, the stepping motion is used as the motion information of the user, and the mat 140 is employed as a device for measuring it. However, the device for measuring the motion information is not limited thereto, and may be a device for measuring the other motion information, or a device combined them. Also, the other method may be employed as the method for detecting the motion. For example, the user moves a controller in which the acceleration sensor is implemented, and the motion of the user is detected on the basis of the acceleration at that time. Also, for example, a camera photographs the motion of the user, and the motion of the user is detected by analyzing the pictures.

(14) In the mail process in step S1017 of FIG. 36, the predetermined operation instruction for entering the exercise mode is displayed on the monitor 134. However, the instruction may be given by voice, or by both of the image and voice. Also, in FIG. 39, the exercise process in step S1409 is performed by the processor 142 by executing the program stored in the external memory 144. However, the center server 93 may perform the process thereof, and the image may be displayed on a browser of the monitor 134.

Also, in the above second modification, the predetermined operation instruction for entering the exercise mode instructs to operate the key of the computer 131. However, a device for detecting the motion information may be utilized, e.g., the operation instruction may instruct to tread on the specified foot switch of the mat 140. In this case, when the device for detecting the motion information detects the specified motion, the fact is conveyed to the processor 142, and the processor 142 receives it to enter the exercise mode.

(15) In the above second modification, the health management terminal 97 accesses the center server 93 and acquires the health management information and the medical information. However, these may be transmitted from the center server 93 to the health management terminal 97 by E-mail.

(16) In the above second modification, the body condition evaluation system 95 may be located in each of the plurality of the stores 96, and the health management terminal 97 may be located in each of the plurality of the personal residences 98. Needless to say, there may be the plurality of the medical institution terminals 100.

(18) In the above second modification, the exercise menu included in the health management information and the medical information from the center server 93 may be a menu utilizing the mat 140, or a menu utilizing the other exercise machine. Of course, such exercise machines do not have to be used. Also, they may be combined. Further, the health management information and the medical information may include the advice and menu of the meal.

(19) The part of the data items to be included in the health management information and the medical information which the center server 93 transmits to the health management terminal 97 may preliminarily be stored in the external memory 144 of the computer 131 of the health management terminal 97. Of course, the computer 131 may be provided with a mass storage device such as a hard disk, and the data may be stored therein. For example, the animations of all exercises which can be included in the exercise menu are preliminarily stored in the computer 131. The center server 93 selects ones suitable for the user from among the animations stored in the computer 131 to created the exercise menu. Accordingly, the center server 93 only has to indicate the animation to be reproduced to the computer 131 by the created exercise menu. Since the computer 131 stores the animations, the download and the streaming are not required. Therefore, even the low-performance processor 142 is implemented due to reduce the cost, it is possible to easily accommodate. Of course, the data to be preliminarily stored in the computer 131 is not limited to the animation.

(20) In the above second modification, the body condition evaluation system 95 according to the first modification is employed. However, the body condition evaluation system according to the above embodiment may be employed.

(21) As physically viewed, the computer (7, 131, 93, 100, and 95) can be implemented with a single computer. Alternatively, the processes of the computer can be performed by a plurality of computers as distributed processing. Of course, in the case where distributed processing is employed, the respective computers may be located in the same country, or distributed in a plurality of countries.

(22) The process steps expressing the programs for making a computer perform various processes do not necessarily have to be executed in time series in the order shown by the flowcharts, and may include processes which are executed in parallel or individually.

(23) The computer programs for executing the processes shown by the above flowcharts may be handled by a single computer, or may be performed by a plurality of computers as distributed processing.

(24) The term “unit” as used in the present specification and claims does not always refer to a physical device but can also refer to software for implementing the functions of this unit. Furthermore, the functions of one unit may be implemented by two or more physical devices. Conversely, the functions of two or more units may be implemented by one physical device.

(25) The present invention measures and evaluates the condition of the body of the subject, provides the exercise prescription or the exercise menu for correcting the posture and so on, and therefore is useful in fields of beauty, health, and medical care.

While the present invention has been described in terms of embodiments, those skilled in the art will recognize that the invention is not limited to the embodiments described. The present invention can be practiced with modification and alteration within the spirit and scope of the appended claims.

1. A body condition evaluation apparatus comprising:
a vicarious movement measuring unit operable to measure vicarious movement accompanying predetermined motion of a subject; and
an evaluating unit operable to evaluate physical condition of a body of the subject on the basis of information of the vicarious movement measured by said vicarious movement measuring unit.

2. The body condition evaluation apparatus as claimed in claim 1, wherein said vicarious movement measuring unit is mounted on the subject so as not to positively restrict the predetermined motion and the vicarious movement.

3. The body condition evaluation apparatus as claimed in claim 2, wherein said vicarious movement measuring unit is arranged on a central line which divides the subject into right and left.

4. The body condition evaluation apparatus as claimed in claim 3, wherein said vicarious movement measuring unit is arranged on any one of a breast, a roughly position of a navel, a position between right and left blade bones, and a lower back of the subject.

5. The body condition evaluation apparatus as claimed in claim 1, wherein the predetermined motion includes a first motion and a second motion which is symmetrical to the first motion with regard to a central line which divides the subject into right and left,
wherein the vicarious movement includes a first vicarious movement accompanying the first motion and a second vicarious movement accompanying the second motion, and
wherein said vicarious movement measuring unit measures the first vicarious movement and the second vicarious movement,
said body condition evaluation apparatus further comprising:
a difference calculating unit operable to calculate a difference between information of the first vicarious movement and information of the second vicarious movement,
wherein the evaluating unit evaluates the physical condition of the body of the subject on the basis of the difference.

6. A body condition evaluation apparatus comprising:
a vicarious movement measuring unit operable to measure vicarious movement accompanying predetermined motion of a subject;
a motion measuring unit operable to measure the predetermined motion including the vicarious movement;
a motion calculating unit operable to calculate motion performed by a part which should originally perform the predetermined motion by subtracting information of the vicarious movement as measured by said vicarious movement measuring unit from information of the predetermined motion as measured by said motion measuring unit; and
an evaluating unit operable to evaluate physical condition of a body of the subject on the basis of information of the motion as calculated by said motion calculating unit.

7. The body condition evaluation apparatus as claimed in claim 6, wherein said vicarious movement measuring unit and said motion measuring unit are mounted on the subject so as not to positively restrict the predetermined motion including the vicarious movement.

8. The body condition evaluation apparatus as claimed in claim 7, wherein said vicarious movement measuring unit and said motion measuring unit are arranged at positions different from each other on a central line which divides the subject into right and left.

9. The body condition evaluation apparatus as claimed in claim 8, wherein said vicarious movement measuring unit is arranged on any one of a breast, a roughly position of a navel, a position between right and left blade bones, and a lower back of the subject, and
wherein said motion measuring unit is arranged at any one of a roughly position of the navel and the lower back of the subject when said vicarious movement measuring unit is arranged on either the breast or the position between the right and left blade bones, and is arranged on any one of the breast and a position between the right and left blade bones of the subject when said vicarious movement measuring unit is arranged at either the roughly position of the navel or the lower back.

10. The body condition evaluation apparatus as claimed in claim 6, wherein the predetermined motion includes a first motion and a second motion which is symmetrical to the first motion with regard to a central line which divides the subject into right and left,
wherein the vicarious movement includes a first vicarious movement accompanying the first motion and a second vicarious movement accompanying the second motion, and
wherein said vicarious movement measuring unit measures the first vicarious movement and the second vicarious movement,
wherein said motion measuring unit measures the first motion including the first vicarious movement and the second motion including the second vicarious movement, and
wherein said motion calculating unit calculate motion performed by a part which should originally perform the first motion by subtracting information of the first vicarious movement from information of the first motion, and motion performed by a part which should originally perform the second motion by subtracting information of the second vicarious movement from information of the second motion,
said body condition evaluation apparatus further comprising:
a difference calculating unit operable to calculate a difference between the motion performed by the part which should originally perform the first motion and the motion performed by the part which should originally perform the second motion,
wherein the evaluating unit evaluates the physical condition of the body of the subject on the basis of the difference as calculated by said difference calculating unit.

11. A body condition evaluation apparatus capable of evaluating physical condition of a body of a subject which has symmetrical structure with regard to a central line, comprising:
a first detecting unit and a second detecting unit configured to be mounted on two parts which are symmetrical to each other with regard to the central line, and detect motions of the parts;
a third detecting unit configured to be mounted on a part on the central line, and detect motion of the part;
a first calculating unit operable to calculate an amount of change of the motion of the corresponding part which is detected by said first detecting unit when the two parts change from a first state to a second state respectively;
a second calculating unit operable to calculate an amount of change of the motion of the corresponding part which is detected by said second detecting unit when the two parts change from the first state to the second state respectively;
a third calculating unit operable to calculate a maximum value and/or a trajectory of the motion of the part which is detected by said third detecting unit from a start to a finish of a predetermined motion which is performed by the subject; and
an evaluating unit operable to evaluate the physical condition of the body of the subject on the basis of the amount of the change calculated by said first calculating unit, the amount of the change calculated by said second calculating unit, and the maximum value and/or the trajectory calculated by said third calculating unit.

12. The body condition evaluation apparatus as claimed in claim 11, wherein the subject is a human,
wherein said first detecting unit is mounted between a shoulder joint and a cubital joint on a left arm of the human,
wherein said second detecting unit is mounted between a shoulder joint and a cubital joint on a right arm of the human, and
wherein the third detecting unit is mounted near a pelvis on a side of a belly or a side of a back of the human.

13. The body condition evaluation apparatus as claimed in claim 12, wherein said evaluating unit comprising:
a first comparing unit operable to compare the amount of the change calculated by said first calculating unit and the amount of the change calculated by said second calculating unit to determine which of a right shoulder and a left shoulder of the subject is higher than the other;
a second comparing unit operable to compare the maximum value in a right direction and the maximum value in a left direction of the subject as calculated by said third calculating unit to determine to which of a right side and a left side a load of the subject is applied;
a third comparing unit operable to compare the maximum value in a forward-tilt direction of the subject as calculated by said third calculating unit and a predetermined value to determine to which of a condition of a forward tilt and a condition of a backward tilt the pelvis belongs; and
a unit operable to evaluate the physical condition of the body of the subject on the basis of the comparison result by said first comparing unit, the comparison result by said second comparing unit, and the comparison result by said third comparing unit.

14. The body condition evaluation apparatus as claimed in claim 11 further comprising:
a change displaying unit operable to display processes of the motions of the two parts which are symmetrical to each other with regard to the central line, the maximum value which changes from moment to moment with regarding to the part on the central line, and/or the trajectory of the motion of the part on the central line with an image on a display device.

15. The body condition evaluation apparatus as claimed in claim 11 further comprising:
a first guiding unit operable to guide a motion from the first state to the second state with an image; and
a second guiding unit operable to guide the predetermined motion with an image.

16. The body condition evaluation apparatus as claimed in claim 11 further comprising:
a correction exercise displaying unit operable to display guidance of exercise for correcting the physical condition of the body indicated by said evaluating unit with an image on a display device.

17. The body condition evaluation apparatus as claimed in claim 11, wherein arbitrary one of an acceleration sensor, an angular velocity sensor, a direction sensor, and an inclination sensor can optionally be employed as said first detecting unit, said second detecting unit, or said third detecting unit.

18. A body condition evaluation apparatus capable of evaluating physical condition of a body of a subject which has symmetrical structure with regard to a central line, comprising:
a first detecting unit and a second detecting unit configured to be mounted on two parts which are symmetrical to each other with regard to the central line, and detect motions of the parts;
a first change amount calculating unit operable to calculate an amount of change of the motion of the corresponding part which is detected by said first detecting unit when the two parts change from a first state to a second state respectively;
a second change amount calculating unit operable to calculate an amount of change of the motion of the corresponding part which is detected by said second detecting unit when the two parts change from the first state to the second state respectively; and
an evaluating unit operable to evaluate the physical condition of the body of the subject on the basis of the amount of the change calculated by said first change amount calculating unit, and the amount of the change calculated by said second change amount calculating unit.

19. A body condition evaluation apparatus capable of evaluating physical condition of a body of a subject which has symmetrical structure with regard to a central line, comprising:
a detecting unit configured to be mounted on a part on the central line, and detect motion of the part;
a maximum value calculating unit operable to calculate a maximum value of the motion of the part which is detected by said detecting unit from a start to a finish of a predetermined motion which is performed by the subject; and
an evaluating unit operable to evaluate the physical condition of the body of the subject on the basis of the maximum value calculated by said maximum value calculating unit.

20. A body condition evaluation apparatus capable of evaluating physical condition of a body of a subject which has symmetrical structure with regard to a central line, comprising:
a detecting unit configured to be mounted on a part on the central line, and detect motion of the part;
a trajectory calculating unit operable to calculate a trajectory of the motion of the part which is detected by said detecting unit from a start to a finish of a predetermined motion which is performed by the subject; and
an evaluating unit operable to evaluate the physical condition of the body of the subject on the basis of the trajectory calculated by said trajectory calculating unit.

21-28. (canceled)
